(19) United States
(12) Patent Application Publication
(10) Pub. No.: US 2013/0079373 A1
(43) Pub. Date: Mar. 28, 2013

(54) Substituted Methanesulfonamide Derivatives as Vanilloid Receptor Ligands

(71) Applicant: Gruenenthal GmbH, Aachen (DE)

(72) Inventors: Robert FRANK, Aachen (DE); Gregor BAHRENBERG, Monschau-Konzen (DE); Thomas CHRISTOPH, Aachen (DE); Bernhard LESCH, Aachen (DE); Jeewoo LEE, Seoul (KR)

(21) Appl. No.: 13/626,329

(22) Filed: Sep. 25, 2012

Related U.S. Application Data
(60) Provisional application No. 61/539,159, filed on Sep. 26, 2011.

(30) Foreign Application Priority Data
Sep. 26, 2011 (EP) 11 007 806.0

(51) Int. Cl. A61K 31/44; C07D 213/64; C07D 405/12; A61K 31/4433; A61P 25/00; A61P 25/06; A61P 25/24; A61P 25/28; A61P 25/08; A61P 11/00; A61P 11/14; A61P 13/00; A61P 13/10; A61P 1/00; A61P 1/04; A61P 27/02; A61P 17/00; A61P 37/08; A61P 17/06; A61P 31/22; A61P 29/00; A61P 1/12; A61P 17/04; A61P 19/10; A61P 19/02; A61P 3/00; A61P 25/30; A61P 25/32; A61P 7/10; A61P 7/00; A61P 9/00; A61P 17/02; A61P 15/00; A61P 25/22; A61P 23/02; A61P 25/16; A61P 25/14; A61P 11/06; A61P 11/08; C07D 213/56

(52) U.S. Cl. 514/336; 546/337; 514/357; 546/291; 514/346; 546/282.1

(57) ABSTRACT

The invention relates to substituted methanesulfonamide derivatives as vanilloid receptor ligands, to pharmaceutical compositions containing these compounds and also to these compounds for use in the treatment and/or prophylaxis of pain and further diseases and/or disorders.

This application claims priority from co-pending U.S. provisional patent application No. 61/539,159, filed Sep. 26, 2011, the entire disclosure of which is incorporated herein by reference. Priority is also claimed based on European patent application no. EP 11 007 806.0, filed Sep. 26, 2011, the entire disclosure of which is likewise incorporated herein by reference.

The invention relates to substituted methanesulfonamide derivatives as vanilloid receptor ligands, to pharmaceutical compositions containing these compounds and also to these compounds for use in the treatment and/or prophylaxis of pain and further diseases and/or disorders.

The treatment of pain, in particular of neuropathic pain, is very important in medicine. There is a worldwide demand for effective pain therapies. The urgent need for action for a patient-focused and target-oriented treatment of chronic and non-chronic states of pain, this being understood to mean the successful and satisfactory treatment of pain for the patient, is also documented in the large number of scientific studies which have recently appeared in the field of applied analgesics or basic research on nociception.

The subtype 1 vanilloid receptor (VR1/TRPV1), which is often also referred to as the capsaicin receptor, is a suitable starting point for the treatment of pain, in particular of pain selected from the group consisting of acute pain, chronic pain, neuropathic pain and visceral pain. This receptor is stimulated inter alia by vanilloids such as capsaicin, heat and protons and plays a central role in the formation of pain. In addition, it is important for a large number of further physiological and pathophysiological processes and is a suitable target for the therapy of a large number of further disorders such as, for example, migraine, depression, neurodegenerative diseases, cognitive disorders, states of anxiety, epilepsy, coughs, diarrhoea, pruritus, inflammations, disorders of the cardiovascular system, eating disorders, medication dependency, misuse of medication and urinary incontinence.

There is a demand for further compounds having comparable or better properties, not only with regard to affinity to vanilloid receptors 1 (VR1/TRPV1 receptors) per se (potency, efficacy).

Thus, it may be advantageous to improve the metabolic stability, the solubility in aqueous media or the permeability of the compounds. These factors can have a beneficial effect on oral bioavailability or can alter the PK/PD (pharmacokinetic/pharmacodynamic) profile; this can lead to a more beneficial period of effectiveness, for example.

It was therefore an object of the invention to provide novel compounds, preferably having advantages over the prior-art compounds. The compounds should be suitable in particular as pharmacological active ingredients in pharmaceutical compositions, preferably in pharmaceutical compositions for the treatment and/or prophylaxis of disorders or diseases which are at least partially mediated by vanilloid receptors 1 (VR1/TRPV1 receptors).

This object is achieved by the subject matter described herein.

It has surprisingly been found that the substituted compounds of general formula (I), as given below, display outstanding affinity to the subtype 1 vanilloid receptor (VR1/TRPV1 receptor) and are therefore particularly suitable for the prophylaxis and/or treatment of disorders or diseases which are at least partially mediated by vanilloid receptors 1 (VR1/TRPV1).

The present invention therefore relates to a substituted compound of general formula (I)

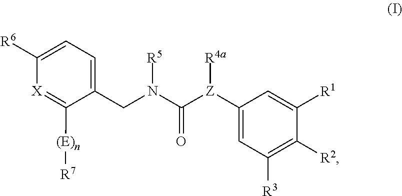

wherein
one of residues R¹ and R² denotes CH₂—N(R⁸)—S(═O)₂—R⁹,
   wherein R⁸ represents H, CH₃ or C₂H₅, and
   wherein R⁹ represents NH₂, CH₃ or C₂H₅,
      and the respective remaining residue of R¹ and R² is selected from the group consisting of H, F, Cl, Br, I, CH₃, CH₂—OH, CH₂—CH₂—OH, CH₂—O—CH₃, CH₂—CH₂—O—CH₃, CF₃, OH, O—CH₃, O—CH₂—OH, O—CH₂—O—CH₃, O—C₂H₅, O—CH₂—CH₂—OH, O—CH₂—CH₂—O—CH₃ and NH₂,
R³ is selected from the group consisting of H, F, Cl, Br, I, CH₃, CF₃, OH, O—CH₃, O—CF₃, and NH₂,
Z represents N or C—R^{4b},
   wherein R^{4b} represents H or CH₃,
R^{4a} represents H or CH₃,
R⁵ represents H or CH₃,
X represents N or CH;
R⁶ represents CF₃, an unsubstituted, saturated C₁₋₄ aliphatic residue or an unsubstituted, saturated C₃₋₆ cycloaliphatic residue,
n denotes 0 or 1,
E represents a C₁₋₄ aliphatic group, (C₁₋₄ aliphatic group)-O, (C₁₋₄ aliphatic group)-O—(C₁₋₄ aliphatic group), (C₁₋₄ aliphatic group)-O—(C₁₋₄ aliphatic group)-O, O, an O—C₁₋₄ aliphatic group, O—(C₁₋₄ aliphatic group)-O, O—(C₁₋₄ aliphatic group)-S, S, a S—C₁₋₄ aliphatic group, S—(C₁₋₄ aliphatic group)-S, or S—(C₁₋₄ aliphatic group)-O,
R⁷ represents a C₁₋₄ aliphatic residue, wherein the C₁₋₄ aliphatic residue can be unsubstituted or mono-, di- or trisubstituted with 1, 2 or 3 substituents selected independently of one another from the group consisting of F, Cl, Br, I, OH, O—CH₃, O—CH₂—OH, O—CH₂—O—CH₃, O—C₂H₅, O—CH₂—CH₂—OH, O—CH₂—CH₂—O—CH₃, O—CF₃, NH₂, NH(CH₃), and N(CH₃)₂, preferably, wherein the C₁₋₄ aliphatic residue can be unsubstituted or monosubstituted with OH and O—CH₃;
   a C₃₋₆ cycloaliphatic residue or a 3 to 6 membered heterocycloaliphatic residue, in each case unsubstituted or mono-, or di-, or trisubstituted with 1, 2 or 3 substituents selected independently of one another from the group consisting of F, Cl, Br, I, CH₃, C₂H₅, CH₂—OH, CH₂—CH₂—OH, CH₂—O—CH₃, CH₂—CH₂—O—CH₃, CH₂—NH(CH₃), CH₂—N(CH₃)₂, CF₃, OH, O—CH₃, O—CH₂—OH, O—CH₂—O—CH₃, O—C₂H₅, O—CH₂—CH₂—OH, O—CH₂—CH₂—O—CH₃, NH₂, NH(CH₃), and N(CH₃)₂, preferably in each case unsubstituted or monosubstituted with F, Cl, Br, I, CH₃, OH or OCH₃, and wherein said C₃₋₆ cycloaliphatic residue and said 3 to 6 membered heterocycloaliphatic residue can in each case optionally be condensed with an unsubstituted phenyl,
   phenyl, or a 5 or 6 membered monocyclic heteroaryl, in each case independently of one another unsubstituted or mono-, or di- or trisubstituted with 1, 2 or 3 substituents selected independently of one another from the group consisting of F, Cl, Br, I, CH₃, C₂H₅, CH₂—OH, CH₂—CH₂—OH, CH₂—O—CH₃, CH₂—CH₂—O—CH₃, CH₂—NH(CH₃), CH₂—N(CH₃)₂, CF₃, OH, O—CH₃, O—CH₂—OH, O—CH₂—O—CH₃, O—C₂H₅, O—CH₂—CH₂—OH, O—CH₂—CH₂—O—CH₃, O—CF₃, SH, S—CH₃, S—CF₃, NH₂, NH(CH₃), and N(CH₃)₂,
      with the proviso that n is 1, if R⁷ represents phenyl, a 6 membered monocyclic heteroaryl or a 3 to 6 membered heterocycloaliphatic residue;
   or a phenyl, which is condensed with a further ring selected from the group consisting of a C₃₋₆ cycloaliphatic residue, a 3 to 6 membered heterocycloaliphatic residue, a phenyl and a 5 or 6 membered monocyclic heteroaryl to form a bicyclic ring system, wherein said ring system is unsubstituted or mono-, or di- or trisubstituted with 1, 2 or 3 substituents selected independently of one another from the group consisting of F, Cl, Br, I, CH₃, C₂H₅, CH₂—OH, CH₂—CH₂—OH, CH₂—O—CH₃, CH₂—CH₂—O—CH₃, CH₂—NH(CH₃), CH₂—N(CH₃)₂, CF₃, OH, O—CH₃, O—CH₂—OH, O—CH₂—O—CH₃, O—C₂H₅, O—CH₂—CH₂—OH, O—CH₂—CH₂—O—CH₃, O—CF₃, S—CF₃, NH₂, NH(CH₃), and N(CH₃)₂;
      in which an “aliphatic group” and an “aliphatic residue” can in each case, independently of one another, be branched or unbranched, saturated or unsaturated, if not indicated otherwise;
      in which a “cycloaliphatic residue” and a “heterocycloaliphatic residue” can in each case, independently of one another, be saturated or unsaturated, if not indicated otherwise;
      optionally in the form of a single stereoisomer or a mixture of stereoisomers, in the form of the free compound and/or a physiologically acceptable salt thereof.

Preferably, when n is 1 and E represents a radical selected from the group comprising (C₁₋₄ aliphatic group)-O, (C₁₋₄ aliphatic group)-O—(C₁₋₄ aliphatic group)-O, O, O—(C₁₋₄ aliphatic group)-O, O—(C₁₋₄ aliphatic group)-S, S, S—(C₁₋₄ aliphatic group)-S, and S—(C₁₋₄ aliphatic group)-O, and R⁷ denotes a 3 to 6 membered heterocycloaliphatic residue or denotes a 5 or 6 membered monocyclic heteroaryl, said 3 to 6 membered heterocycloaliphatic residue or said 5 or 6 membered monocyclic heteroaryl, respectively, is bound to the O or S atom of these radicals via a carbon atom of the 3 to 6 membered heterocycloaliphatic residue and the 5 or 6 membered monocyclic heteroaryl heteroaryl, respectively.

The term “single stereoisomer” comprises in the sense of this invention an individual enantiomer or diastereomer. The term “mixture of stereoisomers” comprises in the sense of this invention the racemate and mixtures of enantiomers and/or diastereomers in any mixing ratio.

The term “physiologically acceptable salt” comprises in the sense of this invention a salt of at least one compound according to the present invention and at least one physiologically acceptable acid or base.

The term “C₁₋₄ aliphatic residue” comprises in the sense of this invention acyclic saturated or unsaturated aliphatic hydrocarbon residues, which can be branched or unbranched and also unsubstituted or mono- or polysubstituted if not indicated otherwise, which contain 1 to 4 carbon atoms (i.e. 1, 2, 3 or 4 carbon atoms) respectively, i.e. C₁₋₄ alkanyls (C₁₋₄ alkyls), C₂₋₄ alkenyls and C₂₋₄ alkynyls, respectively. Alkenyls comprise at least one C—C double bond (a C═C-bond) and alkynyls comprise at least one C—C triple bond (a C≡C-bond). Preferably, aliphatic residues are selected from the group consisting of alkanyl (alkyl) and alkenyl residues, more preferably are alkanyl (alkyl) residues. Preferred C₁₋₄ alkanyl residues are selected from the group consisting of methyl, ethyl, n-propyl, 2-propyl, n-butyl, isobutyl, sec.-butyl, and tert.-butyl. Preferred C₂₋₄ alkenyl residues are selected from the group consisting of ethenyl (vinyl), propenyl (—CH₂CH═CH₂, —CH═CH—CH₃, —C(═CH₂)—CH₃) and butenyl. Preferred C₂₋₄ alkynyl residues are selected from the group consisting of ethynyl, propynyl (—CH₂—C≡CH, —C≡C—CH₃) and butynyl.

The term “C₃₋₆ cycloaliphatic residue” means for the purposes of this invention cyclic aliphatic hydrocarbons containing 3, 4, 5 or 6 carbon atoms, wherein the hydrocarbons in each case can be saturated or unsaturated (but not aromatic), unsubstituted or mono- or polysubstituted, if not indicated otherwise. The cycloaliphatic residues can be bound to the respective superordinate general structure via any desired and possible ring member of the cycloaliphatic residue. Preferred C₃₋₆ cycloaliphatic residues are selected from the group consisting of cyclopropyl, cyclobutyl, cyclopentyl, cyclohexyl, cyclopentenyl, and cyclohexenyl. Particularly preferred C₃₋₆ cycloaliphatic residues are C₆₋₆ cycloaliphatic residues such as cyclopentyl, cyclohexyl, cyclopentenyl and cyclohexenyl.

The term “3-6-membered heterocycloaliphatic residue” means for the purposes of this invention heterocycloaliphatic saturated or unsaturated (but not aromatic) residues having 3-6, i.e. 3, 4, 5 or 6 ring members, in which in each case at least one, if appropriate also two or three carbon atoms are replaced by a heteroatom or a heteroatom group each selected independently of one another from the group consisting of O, S, S(═O)₂, N, NH and N(C₁₋₈ alkyl) such as N(CH₃), preferably are replaced by a heteroatom or a heteroatom group each selected independently of one another from the group consisting of O, S, N, NH and N(C₁₋₈ alkyl) such as N(CH₃), wherein the ring members can be unsubstituted or mono- or polysubstituted, if not indicated otherwise. The heterocycloaliphatic residue can be bound to the superordinate general structure via any desired and possible ring member of the heterocycloaliphatic residue if not indicated otherwise. Preferred heterocycloaliphatic residues are selected from the group consisting of azetidinyl, aziridinyl, dithiolanyl, dihydropyrrolyl, dioxanyl, dioxolanyl, dihydropyridinyl, dihydrofuranyl, dihydroisoxazolyl, dihydrooxazolyl, imidazolidinyl, isoxazolidinyl, oxazolidinyl, morpholinyl, oxiranyl, oxetanyl, pyrrolidinyl, piperazinyl, 4-methylpiperazinyl, piperidinyl, pyrazolidinyl, pyranyl, tetrahydropyrrolyl, tetrahydropyranyl, in particular tetrahydro-2H-pyran-4-yl, tetrahydrofuranyl, tetrahydropyridinyl, tetrahydrothiophenyl, tetrahydroisoxazolyl, thiazolidinyl and thiomorpholinyl.

Radical R⁷ can denote a C₃₋₆ cycloaliphatic residue or a 3 to 6 membered heterocycloaliphatic residue which in each case may optionally be condensed with an unsubstituted phenyl, e.g. said C₃₋₆ cycloaliphatic residue condensed with a phenyl may form for example a dihydroindenyl or said 3 to 6 membered heterocycloaliphatic residue condensed with a phenyl may form for example an indolyl or isoindolyl.

The term “phenyl” means for the purpose of this invention an aromatic hydrocarbon having 6 ring members. Each phenyl residue can be unsubstituted or mono- or polysubstituted if not indicated otherwise, wherein the phenyl substituents can be the same or different and in any desired and possible position of the phenyl. The phenyl can be bound to the superordinate general structure via any desired and possible ring member of the phenyl residue. A phenyl cannot be condensed with any further ring if not indicated otherwise.

Radical R⁷ can denote a phenyl, which is condensed with a further ring selected from the group consisting of a C₃₋₆ cycloaliphatic residue, a 3 to 6 membered heterocycloaliphatic residue, a phenyl and a 5 or 6 membered monocyclic heteroaryl to form a bicyclic ring system, wherein said ring system is unsubstituted or mono-, or di- or trisubstituted with 1, 2 or 3 substituents as described before, e.g. said phenyl can be condensed with a C₃₋₆ cycloaliphatic residue such as cyclopentyl, or a 3 to 6 membered heterocycloaliphatic residue such as a dioxolanyl or a dihydropyrrolyl, or a 6-membered heteroaryl such as a pyridyl.

The term “heteroaryl” for the purpose of this invention represents a monocyclic 5- or 6-membered aromatic residue containing at least 1, if appropriate also 2, 3, 4 or 5 heteroatoms, wherein the heteroatoms are each selected independently of one another from the group S, N and O and the heteroaryl residue can be unsubstituted or mono- or polysubstituted, if not indicated otherwise; in the case of substitution on the heteroaryl, the substituents can be the same or different and be in any desired and possible position of the heteroaryl. The binding to the superordinate general structure can be carried out via any desired and possible ring member of the heteroaryl residue if not indicated otherwise. Preferred monocyclic 5-membered heteroaryl residues are selected from the group consisting of furyl (furanyl), imidazolyl, isoxazolyl, isothiazolyl, oxazolyl, oxadiazolyl, pyrazolyl, pyrrolyl, thienyl (thiophenyl), triazolyl, tetrazolyl, thiazolyl, and thiadiazolyl. Preferred monocyclic 6-membered heteroaryl residues are selected from the group consisting of pyridyl (2-pyridyl, 3-pyridyl, 4-pyridyl), pyridazinyl, pyrimidinyl, pyrazinyl, and triazinyl.

The term “C₁₋₄ aliphatic group” for the purpose of this invention represents a branched or unbranched, saturated or unsaturated, C₁₋₄ aliphatic group having 1, 2, 3 or 4 carbon atoms, i.e. can be a C₁₋₄ alkylene group, a C₂₋₄ alkenylene group or a C₂₋₄ alkynylene group. Preferably, the C₁₋₄-aliphatic group is a C₁₋₄ alkylene group or a C₂₋₄ alkenylene group, more preferably a C₁₋₄ alkylene group. Preferred C₁₋₄ alkylene groups are selected from the group consisting of —CH₂—, —CH₂—CH₂—, —CH(CH₃)—, —CH₂—CH₂—CH₂—, —CH(CH₃)—CH₂—, —CH(CH₂CH₃)—, —CH₂—(CH₂)₂—CH₂—, —CH(CH₃)—CH₂—CH₂—, —CH₂—CH(CH₃)—CH₂—, —CH(CH₃)—CH(CH₃)—, —CH(CH₂CH₃)—CH₂—, —C(CH₃)₂—CH₂—, —CH(CH₂CH₂CH₃)— and —C(CH₃)(CH₂CH₃)—. Preferred C₂₋₄ alkenylene groups are selected from the group consisting of —CH═CH—, —CH═CH—CH₂—, —C(CH₃)═CH₂—, —CH═CH—CH₂—CH₂—, —CH₂—CH═CH—CH₂—, —CH═CH—CH═CH—, —C(CH₃)═CH—CH₂—, —CH═C(CH₃)—CH₂—, —C(CH₃)═C(CH₃)— and —C(CH₂CH₃)═CH—. Preferred C₂₋₄ alkynylene groups are selected from the group consisting of —C≡C—, —C≡C—CH₂—, —C≡C—CH₂—CH₂—, —C≡C—CH(CH₃)—, —CH₂—C≡C—CH₂— and —C≡C—C≡C—.

In relation to the terms “aliphatic residue”, “aliphatic group”, “cycloaliphatic residue” and “heterocycloaliphatic residue”, the term “substituted” refers in the sense of this invention, with respect to the corresponding residues or groups, to the single substitution or multiple substitution (polysubstitution), e.g. disubstitution or trisubstitution, of one or more hydrogen atoms each independently of one another by at least one substituent. In case of a multiple substitution, i.e. in case of polysubstituted residues, such as di- or trisubstituted residues, these residues may be polysubstituted either on different or on the same atoms, for example trisubstituted on the same carbon atom, as in the case of CF₃, CH₂CF₃ or disubstituted as in the case of 1,1-difluorocyclohexyl, or at various points, as in the case of CH(OH)—CH═CH—CHCl₂ or 1-chloro-3-fluorocyclohexyl. The multiple substitution can be carried out using the same or using different substituents.

In relation to the terms “phenyl” and “heteroaryl”, the term “substituted” refers in the sense of this invention, with respect to the corresponding residues or groups, to the single substitution or multiple substitution (polysubstitution), e.g. disubstitution or trisubstitution, of one or more hydrogen atoms each independently of one another by at least one substituent. The multiple substitution can be carried out using the same or using different substituents.

Within the scope of the present invention, the symbol

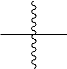

used in the formulae denotes a link of a corresponding residue to the respective superordinate general structure.

If a residue occurs multiply within a molecule, then this residue can have respectively different meanings for various substituents: if, for example, both R⁶ and R⁷ denote a saturated C₁₋₄ aliphatic residue, then the C₁₋₄ aliphatic residue can e.g. represent methyl for R⁶ and can represent ethyl for R⁷.

The terms “salt formed with a physiologically compatible acid” or “salt of physiologically acceptable acids” refers in the sense of this invention to salts of the respective active ingredient with inorganic or organic acids which are physiologically compatible—in particular when used in human beings and/or other mammals. Examples of physiologically acceptable acids are: hydrochloric acid, hydrobromic acid, sulphuric acid, methanesulphonic acid, p-toluenesulphonic acid, carbonic acid, formic acid, acetic acid, oxalic acid, succinic acid, tartaric acid, mandelic acid, fumaric acid, maleic acid, lactic acid, citric acid, glutamic acid, saccharic acid, monomethylsebacic acid, 5-oxoproline, hexane-1-sulphonic acid, nicotinic acid, 2, 3 or 4-aminobenzoic acid, 2,4,6-trimethylbenzoic acid, α-lipoic acid, acetyl glycine, hippuric acid, phosphoric acid, aspartic acid. Citric acid and hydrochloric acid are particularly preferred.

The terms “salt formed with a physiologically compatible base” or “salt of physiologically acceptable bases” refers in the sense of this invention to salts of the respective compound according to the invention—as an anion, e.g. upon deprotonation of a suitable functional group—with at least one cation or base—preferably with at least one inorganic cation—which are physiologically acceptable—in particular when used in human beings and/or other mammals. Particularly preferred are the salts of the alkali and alkaline earth metals, in particular (mono-) or (di)sodium, (mono-) or (di)potassium, magnesium or calcium salts, but also ammonium salts [NHₓR₄₋ₓ]⁺, in which x=0, 1, 2, 3 or 4 and R represents a branched or unbranched C₁₋₄ aliphatic residue.

Further preferred embodiments of the compound according to the invention of general formula (I) have general formulae (I-a) and/or (I-b)

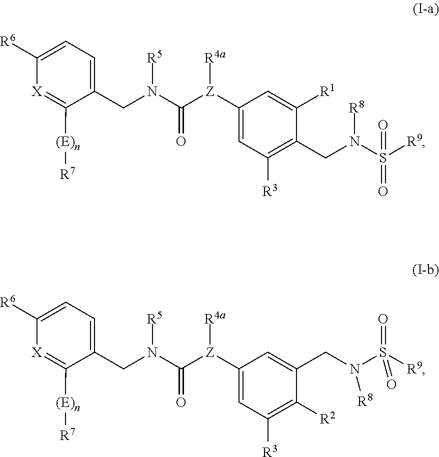

wherein the particular radicals and variables have the meanings described herein in connection with the compounds according to the invention and preferred embodiments thereof. More preferred is an inventive compound according to formula (I-a).

Particularly preferred embodiments of the compound of general formulae (I-a) and (I-b), respectively, have general formulae (I-a-1) and/or (I-b-1), respectively

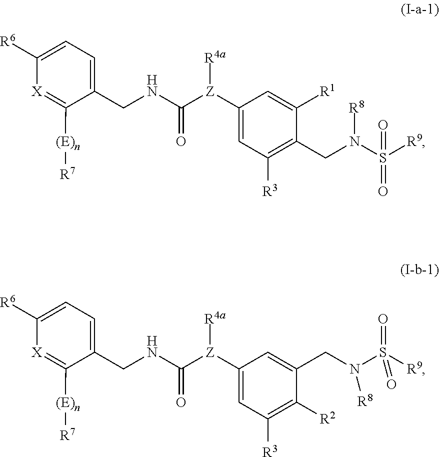

wherein the particular radicals and variables have the meanings described herein in connection with the compounds according to the invention and preferred embodiments thereof. Even more preferred is an inventive compound according to formula (I-a-1).

Most preferred embodiments of the compound of general formulae (I-a-1) and (I-b-1), respectively, have general formulae (I-a-1-a), (I-a-1-b), (I-b-1-a) and (I-b-1-b), respectively

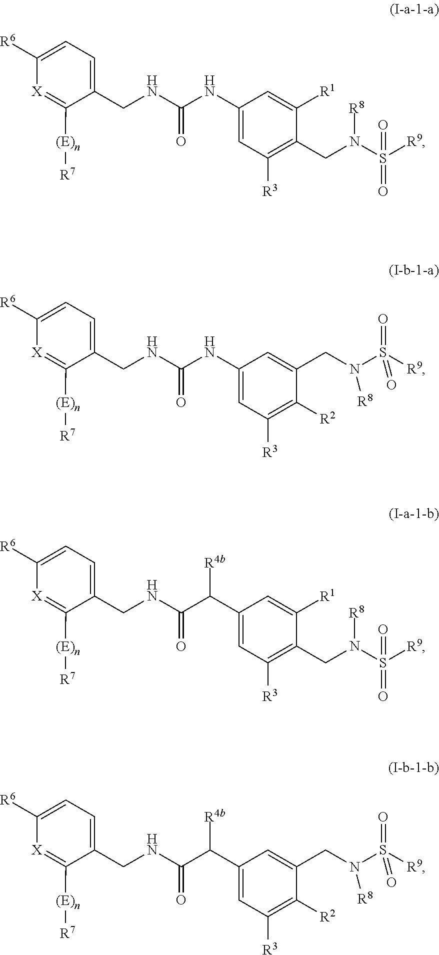

wherein the particular radicals and variables have the meanings described herein in connection with the compounds according to the invention and preferred embodiments thereof. Particularly preferred is an inventive compound according to formula (I-a-1-a) and/or (I-a-1-b).

Further particularly preferred embodiments of the compound of general formulae (I-a) and (I-b), respectively, have general formulae (I-a-1) and/or (I-b-1), respectively

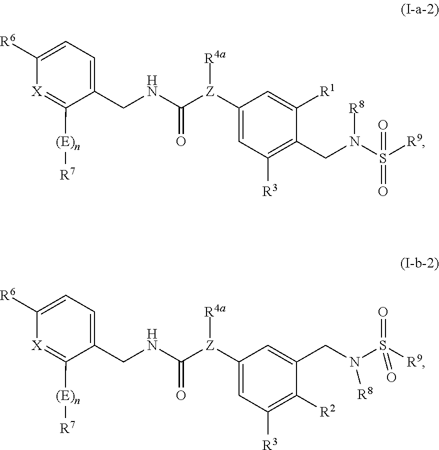

wherein the particular radicals and variables have the meanings described herein in connection with the compounds according to the invention and preferred embodiments thereof. Particularly preferred is an inventive compound according to formula (I-a-2).

Most preferred is a compound of general formula (I) according to formula (I-c) and/or (I-d)

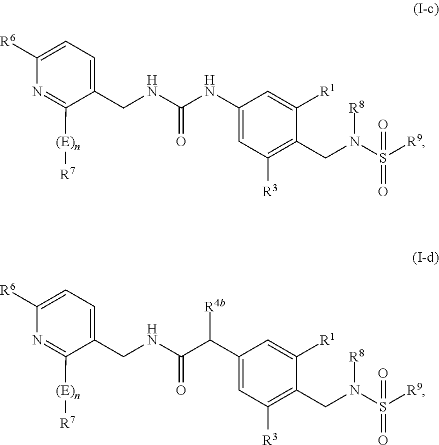

wherein the particular radicals and variables have the meanings described herein in connection with the compounds according to the invention and preferred embodiments thereof.

Further particularly preferred embodiments of the compound of general formulae (I) have one of the following formulae

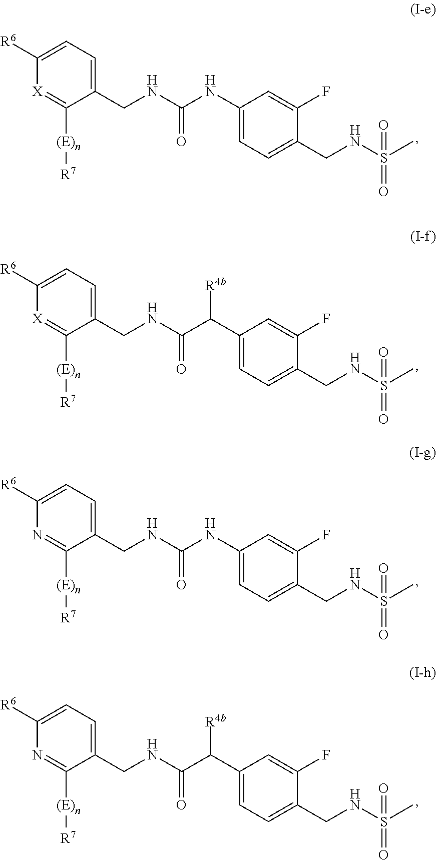

wherein the particular radicals and variables have the meanings described herein in connection with the compounds according to the invention and preferred embodiments thereof.

In a further preferred embodiment of the compound of general formula (I) according to the present invention

one of residues R¹ and R² denotes CH₂—N(R⁸)—S(═O)₂—R⁹,
wherein R⁸ represents H, CH₃, or C₂H₅, and
wherein R⁹ represents NH₂, CH₃, or C₂H₅,
   and the respective remaining residue of R¹ and R² is selected from the group consisting of H, F, Cl, Br, I, CH₃, CH₂—OH, CH₂—O—CH₃, CF₃, OH, and O—CH₃.

Preferably,

one of residues R¹ and R² denotes CH₂—N(R⁸)—S(═O)₂—R⁹,
wherein R⁸ represents H, CH₃, or C₂H₅, and
wherein R⁹ represents NH₂, CH₃, or C₂H₅,
   and the respective remaining residue of R¹ and R² is selected from the group consisting of H, F, Cl, Br, I, CH₃, CF₃, OH, and O—CH₃.

More preferably,

one of residues R¹ and R² denotes CH₂—N(R⁸)—S(═O)₂—R⁹,
wherein R⁸ represents H, CH₃, or C₂H₅, and
wherein R⁹ represents NH₂, CH₃, or C₂H₅,
   and the respective remaining residue of R¹ and R² is selected from the group consisting of H, F, Cl, CH₃, OH, and O—CH₃.

In another preferred embodiment of the compound of general formula (I) according to the present invention
R² denotes CH₂—N(R⁸)—S(═O)₂—R⁹,
   wherein R⁸ represents H, CH₃, or C₂H₅, and
   wherein R⁹ represents NH₂, CH₃, or C₂H₅,
and R¹ is selected from the group consisting of H, F, Cl, Br, I, CH₃, CH₂—OH, CH₂—O—CH₃, CF₃, OH, and O—CH₃.

Preferably,
R² denotes CH₂—N(R⁸)—S(═O)₂—R⁹,
   wherein R⁸ represents H, CH₃, or C₂H₅, and
   wherein R⁹ represents NH₂, CH₃, or C₂H₅,
and R¹ is selected from the group consisting of H, F, Cl, Br, I, CH₃, CF₃, OH, and O—CH₃.

More preferably,
R² denotes CH₂—N(R⁸)—S(═O)₂—R⁹,
   wherein R⁸ represents H, CH₃, or C₂H₅, and
   wherein R⁹ represents NH₂, CH₃, or C₂H₅,
and R¹ is selected from the group consisting of H, F, Cl, CH₃, OH, and O—CH₃.

In yet another preferred embodiment of the compound of general formula (I) according to the present invention
R¹ denotes CH₂—N(R⁸)—S(═O)₂—R⁹,
   wherein R⁸ represents H, CH₃, or C₂H₅, and
   wherein R⁹ represents NH₂, CH₃, or C₂H₅,
and R² is selected from the group consisting of H, F, Cl, Br, I, CH₃, CH₂—OH, CH₂—O—CH₃, CF₃, OH, and O—CH₃.

Preferably,
R¹ denotes CH₂—N(R⁸)—S(═O)₂—R⁹,
   wherein R⁸ represents H, CH₃, or C₂H₅, and
   wherein R⁹ represents NH₂, CH₃, or C₂H₅,
and R² is selected from the group consisting of H, F, Cl, Br, I, CH₃, CF₃, OH, and O—CH₃.

More preferably,
R¹ denotes CH₂—N(R⁸)—S(═O)₂—R⁹,
   wherein R⁸ represents H, CH₃, or C₂H₅, and
   wherein R⁹ represents NH₂, CH₃, or C₂H₅,
and R² is selected from the group consisting of H, F, Cl, CH₃, OH, and O—CH₃.

In a further preferred embodiment of the compound of general formula (I) according to the present invention

R³ is selected from the group consisting of H, F, Cl, CH₃, CF₃, OH and O—CH₃.

Preferably,

R³ is selected from the group consisting of H, F, Cl, CH₃, and O—CH₃.

More preferably,
R³ is selected from the group consisting of H, F, and Cl, even more preferably denotes H or F, in particular H.

In another preferred embodiment of the compound of general formula (I) according to the present invention

Z represents N and
R^{4a} represents H,
or
Z represents C—R^{4b},
wherein R^{4b} represents H or CH₃, and
   R^{4a} represents H.

In yet another preferred embodiment of the compound of general formula (I) according to the present invention

Z represents N and
R^{4a} represents H,
or
Z represents C—R^{4b}
wherein R^{4b} represents H, and
   R^{4a} represents H or CH₃.

In a further preferred embodiment of the compound of general formula (I) according to the present invention,

Z represents N and R^{4a} represents H; or
Z represents CR^{4b} and R^{4a} and R^{4b} each represent H; or
Z represents CR^{4b} and R^{4a} represents methyl and R^{4b} represents H; or
Z represents CR^{4b} and R^{4a} represents H and R^{4b} represents methyl.

In another preferred embodiment of the compound of general formula (I) according to the present invention

R⁵ represents H.

In a further preferred embodiment of the compound of general formula (I) according to the present invention

X represents N.

In another preferred embodiment of the compound of general formula (I) according to the present invention

X represents CH.

In a further preferred embodiment of the compound of general formula (I) according to the present invention

R⁶ represents CF₃, methyl, ethyl, 2-propyl, isobutyl, sec.-butyl, tert.-butyl, cyclopropyl, cyclobutyl or cyclopentyl.

Preferably,

R⁶ represents CF₃, methyl, ethyl, 2-propyl, tert.-butyl, cyclopropyl, or cyclobutyl.

More preferably,

R⁶ represents CF₃, tert.-Butyl or cyclopropyl.

In another preferred embodiment of the compound of general formula (I) according to the present invention
n denotes 0 or 1,
E represents a C₁₋₄ aliphatic group, (C₁₋₄ aliphatic group)-O, (C₁₋₄ aliphatic group)-O—(C₁₋₄ aliphatic group), (C₁₋₄ aliphatic group)-O—(C₁₋₄ aliphatic group)-O, O, an O—C₁₋₄ aliphatic group, O—(C₁₋₄ aliphatic group)-O, O—(C₁₋₄ aliphatic group)-S, S, a S—C₁₋₄ aliphatic group, S—(C₁₋₄ aliphatic group)-S, or S—(C₁₋₄ aliphatic group)-O,
R⁷ represents a C₁₋₄ aliphatic residue, wherein the C₁₋₄ aliphatic residue can be unsubstituted or mono-, di- or trisubstituted with 1, 2 or 3 substituents selected independently of one another from the group consisting of F, Cl, OH, O—CH₃, O—CH₂—OH, O—CH₂—O—CH₃, O—C₂H₅, O—C₂H₄—OH, and O—C₂H₄—O—CH₃;
   a C₃₋₆ cycloaliphatic residue or a 3 to 6 membered heterocycloaliphatic residue, in each case unsubstituted or mono-, or di-, or trisubstituted with 1, 2 or 3 substituents selected independently of one another from the group consisting of F, Cl, Br, I, CH₃, C₂H₅, CH₂—OH, C₂H₄—OH, CH₂—O—CH₃, C₂H₄—O—CH₃, CF₃, OH, O—CH₃, NH₂, NH(CH₃), and N(CH₃)₂;
   or an unsubstituted C₃₋₆ cycloaliphatic residue or an unsubstituted 3 to 6 membered heterocycloaliphatic residue, which is in each case condensed with an unsubstituted phenyl,
   phenyl, or a 5 or 6 membered monocyclic heteroaryl, in each case independently of one another unsubstituted or mono-, or di- or trisubstituted with 1, 2 or 3 substituents selected independently of one another from the group consisting of F, Cl, Br, I, CH₃, C₂H₅, CH₂—OH, CH₂—O—CH₃, CF₃, OH, O—CH₃, O—CF₃, S—CF₃, NH₂, NH(CH₃), and N(CH₃)₂;
      with the proviso that n is 1, if R⁷ represents phenyl, a 6 membered monocyclic heteroaryl or a 3 to 6 membered heterocycloaliphatic residue;
   or a phenyl, which is condensed with a further ring selected from the group consisting of a C₃₋₆ cycloaliphatic residue, a 3 to 6 membered heterocycloaliphatic residue, a phenyl and a 5 or 6 membered monocyclic heteroaryl to form a bicyclic ring system, wherein said ring system is unsubstituted or mono-, or di- or trisubstituted with 1, 2 or 3 substituents selected independently of one another from the group consisting of F, Cl, Br, I, CH₃, C₂H₅, CH₂—OH, C₂H₄—OH, CH₂—O—CH₃, C₂H₄—O—CH₃, CF₃, OH, O—CH₃, O—CH₂—OH, O—CH₂—O—CH₃, C₂H₄, O—C₂H₄—OH, O—C₂H₄—O—CH₃, O—CF₃, S—CF₃, NH₂, NH(CH₃), and N(CH₃)₂;

Preferably,
n denotes 0 or 1,
E is selected from the group consisting of CH₂, CH₂—CH₂, CH₂—CH₂—CH₂, CH═CH, C≡C, CH₂—O, CH₂—CH₂—O, CH₂—CH₂—CH₂—O, CH₂—O—CH₂, CH₂—CH₂—O—CH₂, CH₂—CH₂—CH₂—O—CH₂, CH₂—O—CH₂—CH₂, CH₂—CH₂—O—CH₂—CH₂, CH₂—CH₂—CH₂—O—CH₂—CH₂, CH₂—O—CH₂—CH₂—CH₂, CH₂—CH₂—O—CH₂—CH₂—CH₂, CH₂—CH₂—CH₂—O—CH₂—CH₂—CH₂, CH₂—O—CH₂—O, CH₂—CH₂—O—CH₂—O, CH₂—CH₂—CH₂—O—CH₂—O, CH₂—O—CH₂—CH₂—O, CH₂—CH₂—O—CH₂—CH₂—O, CH₂—CH₂—CH₂—O—CH₂—CH₂—, CH₂—O—CH₂—CH₂—CH₂—O, CH₂—CH₂—O—CH₂—CH₂—CH₂—O, CH₂—CH₂—CH₂—O—CH₂—CH₂—CH₂—O, O, O—CH₂, O—CH₂—CH₂, O—CH₂—CH₂—CH₂, O—CH₂—, O—CH₂—CH₂—O, O—CH₂—CH₂—CH₂—O, O—CH₂—S, O—CH₂—CH₂—S, O—CH₂—CH₂—CH₂—S, S, S—CH₂, S—CH₂—CH₂, S—CH₂—CH₂—CH₂, S—CH₂—O, S—CH₂—CH₂—O, and S—CH₂—CH₂—CH₂—O,
R⁷ represents a C₁₋₄ aliphatic residue, wherein the C₁₋₄ aliphatic residue can be unsubstituted or mono-, di- or trisubstituted with 1, 2 or 3 substituents selected independently of one another from the group consisting of F, Cl, OH, and O—CH₃, preferably wherein the C₁₋₄ aliphatic residue can be unsubstituted or monosubstituted with OH or O—CH₃,
   a C₃₋₆ cycloaliphatic residue or a 3 to 6 membered heterocycloaliphatic residue, in each case unsubstituted or mono-, or di-, or trisubstituted with 1, 2 or 3 substituents selected independently of one another from the group consisting of F, Cl, CH₃, C₂H₅, CH₂—OH, CF₃, OH, O—CH₃, NH₂, NH(CH₃), and N(CH₃)₂, preferably in each case unsubstituted or monosubstituted with F, Cl, Br, I, CH₃, OH or OCH₃;
   or an unsubstituted C₃₋₆ cycloaliphatic residue, which is condensed with an unsubstituted phenyl,
   phenyl, or a 5 or 6 membered monocyclic heteroaryl, in each case independently of one another unsubstituted or mono-, or di- or trisubstituted with 1, 2 or 3 substituents selected independently of one another from the group consisting of F, Cl, CH₃, C₂H₅, CF₃, OH, O—CH₃, and O—CF₃,
      with the proviso that n is 1, if R⁷ represents phenyl, a 6 membered monocyclic heteroaryl or a 3 to 6 membered heterocycloaliphatic residue;
   or a phenyl which is condensed with a further ring selected from the group consisting of a C₃₋₆ cycloaliphatic residue and a 3 to 6 membered heterocycloaliphatic residue to form a bicyclic ring system, wherein said ring system is unsubstituted or mono-, or di- or trisubstituted with 1, 2 or 3 substituents selected independently of one another from the group consisting of F, Cl, CH₃, C₂H₅, CF₃, OH, O—CH₃, and O—CF₃.

More preferably,
n denotes 0 or 1,
E is selected from the group consisting of CH₂, CH₂—CH₂, CH₂—CH₂—CH₂, CH═CH, C≡C, CH₂—O, CH₂—CH₂—O, CH₂—CH₂—CH₂—O, CH₂—O—CH₂, CH₂—CH₂—O—CH₂, CH₂—CH₂—CH₂—O—CH₂, CH₂—O—CH₂—CH₂, CH₂—CH₂—O—CH₂—CH₂, CH₂—CH₂—CH₂—O—CH₂—CH₂, CH₂—O—CH₂—CH₂—CH₂, CH₂—CH₂—O—CH₂—CH₂—CH₂, CH₂—CH₂—CH₂—O—CH₂—CH₂—CH₂, CH₂—O—CH₂—O, CH₂—CH₂—O—CH₂—O, CH₂—CH₂—CH₂—O—CH₂—O, CH₂—O—CH₂—CH₂—O, CH₂—CH₂—O—CH₂—CH₂—O, CH₂—CH₂—CH₂—O—CH₂—CH₂—O, CH₂—O—CH₂—CH₂—CH₂—O, CH₂—CH₂—O—CH₂—CH₂—CH₂—O, CH₂—CH₂—CH₂—O—CH₂—CH₂—CH₂—O, O, O—CH₂, O—CH₂—CH₂, O—CH₂—CH₂—CH₂, O—CH₂—O, O—CH₂—CH₂—O, O—CH₂—CH₂—CH₂—O, O—CH₂—S, O—CH₂—CH₂—S, O—CH₂—CH₂—CH₂—S, S, S—CH₂, S—CH₂—CH₂, S—CH₂—CH₂—CH₂, S—CH₂—O, S—CH₂—CH₂—O, and S—CH₂—CH₂—CH₂—O,
R⁷ represents an unsubstituted C₁₋₄ aliphatic residue, preferably selected from the group consisting of methyl, ethyl, n-propyl, 2-propyl, n-butyl, and tert.-butyl,
   a C₃₋₆ cycloaliphatic residue, preferably selected from the group consisting of cyclopropyl, cyclobutyl, cyclopentyl, cyclohexyl, cyclopentenyl, cyclohexenyl or a 3 to 6 membered heterocycloaliphatic residue, preferably selected from the group consisting of piperidinyl, pyrrolidinyl, piperazinyl, morpholinyl and tetrahydropyranyl, wherein the C₃₋₆ cycloaliphatic residue and 3 to 6 membered heterocycloaliphatic residue can in each case independently of one another be unsubstituted or mono-, or di-, or trisubstituted with 1, 2 or 3 substituents selected independently of one another from the group consisting of F, Cl, CH₃, OH, and O—CH₃;
   an unsubstituted dihydroindenyl,
   phenyl, unsubstituted or mono-, or di- or trisubstituted with 1, 2 or 3 substituents selected independently of one another from the group consisting of F, Cl, CH₃, CF₃, OH, O—CH₃, and O—CF₃,
   or a 5 membered monocyclic heteroaryl, preferably selected from the group consisting of furyl, thienyl, oxazolyl, isooxazolyl and thiazolyl, unsubstituted or mono-, or di- or trisubstituted with 1, 2 or 3 substituents selected independently of one another from the group consisting of F, Cl, CH₃, CF₃, OH, O—CH₃, and O—CF₃,
   or a 6 membered monocyclic heteroaryl, preferably selected from the group consisting of pyridyl and pyrimidinyl, unsubstituted or mono-, or di- or trisubstituted with 1, 2 or 3 substituents selected independently of one another from the group consisting of F, Cl, CH₃, CF₃, OH, O—CH₃, and O—CF₃,
      with the proviso that n is 1, if R⁷ represents phenyl, a 6 membered monocyclic heteroaryl or a 3 to 6 membered heterocycloaliphatic residue;
   or a phenyl, which is condensed with a further ring selected from the group consisting of a C₃₋₆ cycloaliphatic residue and a 3 to 6 membered heterocycloaliphatic residue, to form a bicyclic ring system, preferably a bicyclic system selected from the group consisting of benzodioxolanyl, benzodioxanyl, indolyl and isoindolyl, wherein said ring system is unsubstituted or mono-, or di- or trisubstituted with 1, 2 or 3 substituents selected independently of one another from the group consisting of F, Cl, CH₃, CF₃, OH, O—CH₃, and O—CF₃.

Even more preferably,
n denotes 0 or 1,
E is selected from the group consisting of CH₂, CH₂—CH₂, CH₂—CH₂—CH₂, CH═CH, C≡C, CH₂—O, CH₂—CH₂—O, CH₂—CH₂—CH₂—O, CH₂—O—CH₂, CH₂—CH₂—O—CH₂, CH₂—CH₂—CH₂—O—CH₂, CH₂—O—CH₂—CH₂, CH₂—CH₂—O—CH₂—CH₂, CH₂—CH₂—CH₂—O—CH₂—CH₂, CH₂—O—CH₂—CH₂—CH₂, CH₂—CH₂—O—CH₂—CH₂—CH₂, CH₂—CH₂—CH₂—O—CH₂—CH₂—CH₂, CH₂—O—CH₂—O, CH₂—CH₂—O—CH₂—O, CH₂—CH₂—CH₂—O—CH₂—O, CH₂—O—CH₂—CH₂—O, CH₂—CH₂—O—CH₂—CH₂—O, CH₂—CH₂—CH₂—O—CH₂—CH₂—O, CH₂—O—CH₂—CH₂—CH₂—O, CH₂—CH₂—O—CH₂—CH₂—CH₂—O, CH₂—CH₂—CH₂—O—CH₂—CH₂—CH₂—O, O, O—CH₂, O—CH₂—CH₂, O—CH₂—CH₂—CH₂, O—CH₂—O, O—CH₂—CH₂—O, O—CH₂—CH₂—CH₂—O, O—CH₂—S, O—CH₂—CH₂—S, O—CH₂—CH₂—CH₂—S, S, S—CH₂, S—CH₂—CH₂, S—CH₂—CH₂—CH₂, S—CH₂—O, S—CH₂—CH₂—O, and S—CH₂—CH₂—CH₂—O,
R⁷ represents an unsubstituted C₁₋₄ aliphatic residue, preferably selected from the group consisting of methyl, ethyl, n-propyl, 2-propyl, n-butyl, and tert.-butyl,
   cyclopropyl, cyclobutyl, cyclopentyl, cyclohexyl, cyclopentenyl, cyclohexenyl, dihydroindenyl, piperidinyl, pyrrolidinyl, piperazinyl, morpholinyl or tetrahydropyranyl, in each case independently of one another unsubstituted or mono-, or disubstituted with 1 or 2 substituents selected independently of one another from the group consisting of F, Cl, CH₃, OH, and O—CH₃;

phenyl, unsubstituted or mono-, or di- or trisubstituted with 1, 2 or 3 substituents selected independently of one another from the group consisting of F, Cl, CH₃, CF₃, OH, O—CH₃, and O—CF₃,
furyl, thienyl, oxazolyl, isooxazolyl or thiazolyl, unsubstituted or mono-, or disubstituted with 1 or 2 substituents selected independently of one another from the group consisting of F, Cl, CH₃, CF₃, OH, O—CH₃, and O—CF₃,
or pyridyl or pyrimidinyl, unsubstituted or mono-, or disubstituted with 1 or 2 substituents selected independently of one another from the group consisting of F, Cl, CH₃, CF₃, OH, O—CH₃, and O—CF₃,
   with the proviso that n is 1, if R⁷ represents phenyl, pyridyl, pyrimidinyl, piperidinyl, pyrrolidinyl, piperazinyl, morpholinyl or tetrahydropyranyl;
or a phenyl, which is condensed with a dioxolanyl, dioxanyl, or a dihydropyrrolyl to form a bicyclic ring system selected from the group consisting of benzodioxolanyl, benzodioxanyl, indolyl and isoindolyl, wherein said ring system is unsubstituted.

Still more preferably,
n denotes 0 or 1,
E is selected from the group consisting of CH₂, CH₂—CH₂, CH═CH, C≡C, CH₂—O, CH₂—CH₂—O, CH₂—CH₂—CH₂—O, CH₂—O—CH₂, CH₂—CH₂—O—CH₂, CH₂—CH₂—CH₂—O—CH₂, CH₂—O—CH₂—CH₂, CH₂—CH₂—O—CH₂—CH₂, CH₂—CH₂—CH₂—O—CH₂—CH₂, CH₂—O—CH₂—CH₂—CH₂, CH₂—CH₂—O—CH₂—CH₂—CH₂, CH₂—CH₂—CH₂—O—CH₂—CH₂—CH₂, CH₂—O—CH₂—O, CH₂—CH₂—O—CH₂—O, CH₂—CH₂—CH₂—O—CH₂—O, CH₂—O—CH₂—CH₂—O, CH₂—CH₂—O—CH₂—CH₂—O, CH₂—CH₂—CH₂—O—CH₂—CH₂—O, CH₂—O—CH₂—CH₂—CH₂—O, CH₂—CH₂—O—CH₂—CH₂—CH₂—O, CH₂—CH₂—CH₂—O—CH₂—CH₂—CH₂—O, O, O—CH₂, O—CH₂—CH₂, O—CH₂—CH₂—CH₂, S, S—CH₂, S—CH₂—CH₂, S—CH₂—CH₂—CH₂, S—CH₂—O, S—CH₂—CH₂—O, and S—CH₂—CH₂—CH₂—O, preferably selected from the group consisting of CH₂, CH₂—CH₂, CH═CH, C≡C, CH₂—CH₂—CH₂—O, CH₂—O—CH₂—CH₂—O, O, O—CH₂, O—CH₂—CH₂, O—CH₂—CH₂—CH₂, S, S—CH₂, S—CH₂—CH₂, S—CH₂—CH₂—CH₂, S—CH₂—O, S—CH₂—CH₂—O, and S—CH₂—CH₂—CH₂—O,
R⁷ represents methyl, ethyl, n-propyl, 2-propyl, n-butyl, or tert.-butyl,
   cyclopropyl, cyclopentyl, cyclohexyl, cyclohexenyl, dihydroindenyl, piperidinyl, pyrrolidinyl, morpholinyl or tetrahydropyranyl, in each case independently of one another unsubstituted or mono-, or disubstituted with 1 or 2 substituents selected independently of one another from the group consisting of F, Cl, and CH₃;
   an unsubstituted phenyl,
   furyl, thienyl, oxazolyl, isooxazolyl or thiazolyl, in each case unsubstituted,
      with the proviso that n is 1, if R⁷ represents an unsubstituted phenyl, piperidinyl, pyrrolidinyl, morpholinyl or tetrahydropyranyl;
   or a phenyl, which is condensed with a dioxolanyl or a dihydropyrrolyl to form a bicyclic ring system selected from the group consisting of benzodioxolanyl and indolyl, wherein said ring system is unsubstituted.

Particularly preferred is a compound according to general formula (I), wherein
one of residues R¹ and R² denotes CH₂—N(R⁸)—S(═O)₂—R⁹,
   wherein R⁸ represents H, CH₃, or C₂H₅, and
   wherein R⁹ represents NH₂, CH₃, or C₂H₅,
      and the respective remaining residue of R¹ and R² is selected from the group consisting of H, F, Cl, Br, I, CH₃, CH₂—OH, CH₂—O—CH₃, CF₃, OH, and O—CH₃,
R³ is selected from the group consisting of H, F, Cl, CH₃, and O—CH₃,
Z represents N and
R^{4a} represents H,
or
Z represents C—R^{4b},
   wherein R^{4b} represents H or CH₃, and
R^{4a} represents H,
R⁵ represents H,
X represents N or CH,
R⁶ represents CF₃, tert.-Butyl or cyclopropyl,
n denotes 0 or 1,
E is selected from the group consisting of CH₂, CH₂—CH₂, CH═CH, C≡C, CH₂—O, CH₂—CH₂—O, CH₂—CH₂—CH₂—O, CH₂—O—CH₂, CH₂—CH₂—O—CH₂, CH₂—CH₂—CH₂—O—CH₂, CH₂—O—CH₂—CH₂, CH₂—CH₂—O—CH₂—CH₂, CH₂—CH₂—CH₂—O—CH₂—CH₂, CH₂—O—CH₂—CH₂—CH₂, CH₂—CH₂—O—CH₂—CH₂—CH₂, CH₂—CH₂—CH₂—O—CH₂—CH₂—CH₂, CH₂—O—CH₂—O, CH₂—CH₂—O—CH₂—O, CH₂—CH₂—CH₂—O—CH₂—O, CH₂—O—CH₂—CH₂—O, CH₂—CH₂—O—CH₂—CH₂—O, CH₂—CH₂—CH₂—O—CH₂—CH₂—O, CH₂—O—CH₂—CH₂—CH₂—O, CH₂—CH₂—O—CH₂—CH₂—CH₂—O, CH₂—CH₂—CH₂—O—CH₂—CH₂—CH₂—O, O, O—CH₂, O—CH₂—CH₂, O—CH₂—CH₂—CH₂, S, S—CH₂, S—CH₂—CH₂, S—CH₂—CH₂—CH₂, S—CH₂—O, S—CH₂—CH₂—O, and S—CH₂—CH₂—CH₂—O, preferably selected from the group consisting of CH₂, CH₂—CH₂, CH═CH, C≡C, CH₂—CH₂—CH₂—O, CH₂—O—CH₂—CH₂—O, O, O—CH₂, O—CH₂—CH₂, O—CH₂—CH₂—CH₂, S, S—CH₂, S—CH₂—CH₂, S—CH₂—CH₂—CH₂, S—CH₂—O, S—CH₂—CH₂—O, and S—CH₂—CH₂—CH₂—O,
R⁷ represents methyl, ethyl, n-propyl, 2-propyl, n-butyl, or tert.-butyl,
   cyclopropyl, cyclopentyl, cyclohexyl, cyclohexenyl, dihydroindenyl, piperidinyl, pyrrolidinyl, morpholinyl or tetrahydropyranyl, in each case independently of one another unsubstituted or mono-, or disubstituted with 1 or 2 substituents selected independently of one another from the group consisting of F, Cl, and CH₃;
   an unsubstituted phenyl,
   furyl, thienyl, oxazolyl, isooxazolyl or thiazolyl, in each case unsubstituted,
      with the proviso that n is 1, if R⁷ represents an unsubstituted phenyl, piperidinyl, pyrrolidinyl, morpholinyl or tetrahydropyranyl;
   or a phenyl, which is condensed with a dioxolanyl or a dihydropyrrolyl to form a bicyclic ring system selected from the group consisting of benzodioxolanyl and indolyl, wherein said ring system is unsubstituted.

Particularly preferred are compounds according to the invention from the group
1 2-(3-fluoro-4-(methylsulfonamidomethyl)phenyl)-N-((2-pentyl-6-(trifluoromethyl)pyridin-3-yl)methyl)propanamide;
2 2-(3-fluoro-4-(methylsulfonamidomethyl)phenyl)-N-((2-(3-methoxypropyl)-6-(trifluoromethyl)pyridin-3-yl)methyl)propanamide;
3 2-(3-fluoro-4-(methylsulfonamidomethyl)phenyl)-N-((2-((2-methoxyethoxy)methyl)-6-(trifluoromethyl)pyridin-3-yl)methyl)propanamide;
4 (E)-N-((2-(3,3-dimethylbut-1-enyl)-6-(trifluoromethyl)pyridin-3-yl)methyl)-2-(3-fluoro-4-(methylsulfonamidomethyl)phenyl)propanamide;
5 N-((2-cyclopentyl-6-(trifluoromethyl)pyridin-3-yl)methyl)-2-(3-fluoro-4-(methylsulfonamidomethyl)phenyl)propanamide;
6 N-((2-cyclohexyl-6-(trifluoromethyl)pyridin-3-yl)methyl)-2-(3-fluoro-4-(methylsulfonamidomethyl)phenyl)propanamide;
7 N-((2-(4,4-difluorocyclohexyl)-6-(trifluoromethyl)pyridin-3-yl)methyl)-2-(3-fluoro-4-(methylsulfonamidomethyl)phenyl)propanamide;
8 N-((2-cyclohexenyl-6-(trifluoromethyl)pyridin-3-yl)methyl)-2-(3-fluoro-4-(methylsulfonamidomethyl)phenyl)propanamide;
9 N-((2-(cyclohexylmethyl)-6-(trifluoromethyl)pyridin-3-yl)methyl)-2-(3-fluoro-4-(methylsulfonamidomethyl)phenyl)propanamide;
10 2-(3-fluoro-4-(methylsulfonamidomethyl)phenyl)-N-((2-(piperidin-1-ylmethyl)-6-(trifluoromethyl)pyridin-3-yl)methyl)propanamide;
11 N-((2-benzyl-6-(trifluoromethyl)pyridin-3-yl)methyl)-2-(3-fluoro-4-(methylsulfonamidomethyl)phenyl)propanamide;
12 2-(3-fluoro-4-(methylsulfonamidomethyl)phenyl)-N-((2-phenethyl-6-(trifluoromethyl)pyridin-3-yl)methyl)propanamide;
13 (E)-2-(3-fluoro-4-(methylsulfonamidomethyl)phenyl)-N-((2-styryl-6-(trifluoromethyl)pyridin-3-yl)methyl)propanamide;
14 2-(3-fluoro-4-(methylsulfonamidomethyl)phenyl)-N-((2-(phenylethynyl)-6-(trifluoromethyl)pyridin-3-yl)methyl)propanamide;
15 2-(3-fluoro-4-(methylsulfonamidomethyl)phenyl)-N-((2-isopropoxy-6-(trifluoromethyl)pyridin-3-yl)methyl)propanamide;
16 N-((2-butoxy-6-(trifluoromethyl)pyridin-3-yl)methyl)-2-(3-fluoro-4-(methylsulfonamidomethyl)phenyl)propanamide;
17 N-((2-(cyclopropylmethoxy)-6-(trifluoromethyl)pyridin-3-yl)methyl)-2-(3-fluoro-4-(methylsulfonamidomethyl)phenyl)propanamide;
18 N-((2-(cyclohexylmethoxy)-6-(trifluoromethyl)pyridin-3-yl)methyl)-2-(3-fluoro-4-(methylsulfonamidomethyl)phenyl)propanamide;
19 N-(2-(cyclopentyloxy)-4-(trifluoromethyl)benzyl)-2-(3-fluoro-4-(methylsulfonamidomethyl)phenyl)propanamide;
20 N-((2-(cyclopentyloxy)-6-(trifluoromethyl)pyridin-3-yl)methyl)-2-(3-fluoro-4-(methylsulfonamidomethyl)phenyl)propanamide;
21 N-(4-tert-butyl-2-(cyclopentyloxy)benzyl)-2-(3-fluoro-4-(methylsulfonamidomethyl)phenyl)propanamide;
22 N-((6-tert-butyl-2-(cyclopentyloxy)pyridin-3-yl)methyl)-2-(3-fluoro-4-(methylsulfonamidomethyl)phenyl)propanamide;
23 N-((2-(cyclopentyloxy)-6-cyclopropylpyridin-3-yl)methyl)-2-(3-fluoro-4-(methylsulfonamidomethyl)phenyl)propanamide;
24 N-((2-(2,3-dihydro-1H-inden-2-yloxy)-6-(trifluoromethyl)pyridin-3-yl)methyl)-2-(3-fluoro-4-(methylsulfonamidomethyl)phenyl)propanamide;
25 2-(3-fluoro-4-(methylsulfonamidomethyl)phenyl)-N-((2-(tetrahydro-2H-pyran-4-yloxy)-6-(trifluoromethyl)pyridin-3-yl)methyl)propanamide;
26 2-(3-fluoro-4-(methylsulfonamidomethyl)phenyl)-N-((2-phenoxy-6-(trifluoromethyl)pyridin-3-yl)methyl)propanamide;
27 N-((2-isopropoxy-6-(trifluoromethyl)pyridin-3-yl)methyl)-2-(3-methoxy-4-(methylsulfonamidomethyl)phenyl)propanamide;
28 N-((2-butoxy-6-(trifluoromethyl)pyridin-3-yl)methyl)-2-(3-hydroxy-4-(methylsulfonamidomethyl)phenyl)propanamide;
29 N-((2-butoxy-6-(trifluoromethyl)pyridin-3-yl)methyl)-2-(3-methoxy-4-(methylsulfonamidomethyl)phenyl)propanamide;
30 2-(4-(ethylsulfonamidomethyl)-3-fluorophenyl)-N-((2-isopropoxy-6-(trifluoromethyl)pyridin-3-yl)methyl)propanamide;
31 N-((2-butoxy-6-(trifluoromethyl)pyridin-3-yl)methyl)-2-(4-(ethylsulfonamidomethyl)-3-fluorophenyl)propanamide;
32 1-{[2-isopropoxy-6-(trifluoromethyl)pyridin-3-yl]methyl}-3-{4-[(sulfamoylamino)methyl]phenyl}urea;
33 1-{[2-butoxy-6-(trifluoromethyl)pyridin-3-yl]methyl}-3-{4-[(sulfamoylamino)methyl]phenyl}urea;
34 1-{[2-cyclopentyloxy-6-(trifluoromethyl)pyridin-3-yl]methyl}-3-{4-[(sulfamoylamino)methyl]phenyl}urea;
35 1-{3-fluoro-4-[(sulfamoylamino)methyl]phenyl}-3-{[2-ethoxy-6-(trifluoromethyl)pyridin-3-yl]methyl}urea;
36 1-{3-fluoro-4-[(sulfamoylamino)methyl]phenyl}-3-{[2-isopropoxy-6-(trifluoromethyl)pyridin-3-yl]methyl}urea;
37 1-{3-fluoro-4-[(sulfamoylamino)methyl]phenyl}-3-{[2-butoxy-6-(trifluoromethyl)pyridin-3-yl]methyl}urea;
38 1-{3-fluoro-4-[(sulfamoylamino)methyl]phenyl}-3-{[2-cyclopentyloxy-6-(trifluoromethyl)pyridin-3-yl]methyl}urea;
39 N-((2-(butylthio)-6-(trifluoromethyl)pyridin-3-yl)methyl)-2-(3-fluoro-4-(methylsulfonamidomethyl)phenyl)propanamide;
40 2-(3-fluoro-4-(methylsulfonamidomethyl)phenyl)-N-((2-(3-(4-methylpiperidin-1-yl)propylthio)-6-(trifluoromethyl)pyridin-3-yl)methyl)propanamide;
41 N-((2-(cyclopentylthio)-6-(trifluoromethyl)pyridin-3-yl)methyl)-2-(3-fluoro-4-(methylsulfonamidomethyl)phenyl)propanamide;
42 2-(3-fluoro-4-(methylsulfonamidomethyl)phenyl)-N-((2-(2-phenoxyethylthio)-6-(trifluoromethyl)pyridin-3-yl)methyl)propanamide;
43 N-((2-(cyclohexylthio)-6-(trifluoromethyl)pyridin-3-yl)methyl)-2-(3-fluoro-4-(methylsulfonamidomethyl)phenyl)propanamide;
44 N-((2-(1H-indol-6-yl)-6-(trifluoromethyl)pyridin-3-yl)methyl)-2-(3-fluoro-4-(methylsulfonamidomethyl)phenyl)propanamide;
45 N-((2-(benzo[d][1,3]dioxol-5-yl)-6-(trifluoromethyl)pyridin-3-yl)methyl)-2-(3-fluoro-4-(methylsulfonamidomethyl)phenyl)propanamide;
46 2-(3-fluoro-4-(methylsulfonamidomethyl)phenyl)-N-((2-(furan-3-yl)-6-(trifluoromethyl)pyridin-3-yl)methyl)propanamide;
47 2-(3-fluoro-4-(methylsulfonamidomethyl)phenyl)-N-((2-(thiophen-2-yl)-6-(trifluoromethyl)pyridin-3-yl)methyl)propanamide;
48 2-(3-fluoro-4-(methylsulfonamidomethyl)phenyl)-N-((2-(thiophen-3-yl)-6-(trifluoromethyl)pyridin-3-yl)methyl)propanamide; and
49 2-(3-fluoro-4-(methylsulfonamidomethyl)phenyl)-N-((2-(thiazol-4-yl)-6-(trifluoromethyl)pyridin-3-yl)methyl)propanamide;
   optionally in the form of a single stereoisomer or a mixture of stereoisomers, in the form of the free compound and/or a physiologically acceptable salt thereof.

Furthermore, preference may be given to compounds according to the invention that cause a 50 percent displacement of capsaicin, which is present at a concentration of 100 nM, in a FLIPR assay with CHO K1 cells which were transfected with the human VR1 gene at a concentration of less than 2,000 nM, preferably less than 1,000 nM, particularly preferably less than 300 nM, most particularly preferably less than 100 nM, even more preferably less than 75 nM, additionally preferably less than 50 nM, most preferably less than 10 nM.

In the process, the Ca²⁺ influx is quantified in the FLIPR assay with the aid of a Ca²⁺-sensitive dye (type Fluo-4, Molecular Probes Europe BV, Leiden, the Netherlands) in a fluorescent imaging plate reader (FLIPR, Molecular Devices, Sunnyvale, USA), as described hereinafter.

The substituted compounds according to the invention of the aforementioned general formula (I) and corresponding stereoisomers and also the respective corresponding acids, bases, salts and solvates are toxicologically safe and are therefore suitable as pharmaceutical active ingredients in pharmaceutical compositions.

The present invention therefore further relates to a pharmaceutical composition containing at least one compound according to the invention of the above-indicated formula (I), in each case if appropriate in the form of one of its pure stereoisomers, in particular enantiomers or diastereomers, its racemates or in the form of a mixture of stereoisomers, in particular the enantiomers and/or diastereomers, in any desired mixing ratio, or respectively in the form of a corresponding salt, or respectively in the form of a corresponding solvate, and also if appropriate optionally one or more pharmaceutically compatible auxiliaries.

These pharmaceutical compositions according to the invention are suitable in particular for vanilloid receptor 1-(VR1/TRPV1) regulation, preferably for vanilloid receptor 1-(VR1/TRPV1) inhibition and/or for vanilloid receptor 1-(VR1/TRPV1) stimulation, i.e. they exert an agonistic or antagonistic effect.

Likewise, the pharmaceutical compositions according to the invention are preferably suitable for the prophylaxis and/or treatment of disorders or diseases which are mediated, at least in part, by vanilloid receptors 1.

The pharmaceutical composition according to the invention is suitable for administration to adults and children, including toddlers and babies.

The pharmaceutical composition according to the invention may be found as a liquid, semisolid or solid pharmaceutical form, for example in the form of injection solutions, drops, juices, syrups, sprays, suspensions, tablets, patches, capsules, plasters, suppositories, ointments, creams, lotions, gels, emulsions, aerosols or in multiparticulate form, for example in the form of pellets or granules, if appropriate pressed into tablets, decanted in capsules or suspended in a liquid, and also be administered as much.

In addition to at least one substituted compound of the above-indicated formula (I), if appropriate in the form of one of its pure stereoisomers, in particular enantiomers or diastereomers, its racemate or in the form of mixtures of the stereoisomers, in particular the enantiomers or diastereomers, in any desired mixing ratio, or if appropriate in the form of a corresponding salt or respectively in the form of a corresponding solvate, the pharmaceutical composition according to the invention conventionally contains further physiologically compatible pharmaceutical auxiliaries which can for example be selected from the group consisting of excipients, fillers, solvents, diluents, surface-active substances, dyes, preservatives, blasting agents, slip additives, lubricants, aromas and binders.

The selection of the physiologically compatible auxiliaries and also the amounts thereof to be used depend on whether the pharmaceutical composition is to be applied orally, subcutaneously, parenterally, intravenously, intraperitoneally, intradermally, intramuscularly, intranasally, buccally, rectally or locally, for example to infections of the skin, the mucous membranes and of the eyes. Preparations in the form of tablets, dragées, capsules, granules, pellets, drops, juices and syrups are preferably suitable for oral application; solutions, suspensions, easily reconstitutable dry preparations and also sprays are preferably suitable for parenteral, topical and inhalative application. The substituted compounds according to the invention used in the pharmaceutical composition according to the invention in a repository in dissolved form or in a plaster, agents promoting skin penetration being added if appropriate, are suitable percutaneous application preparations. Orally or percutaneously applicable preparation forms can release the respective substituted compound according to the invention also in a delayed manner.

The pharmaceutical compositions according to the invention are prepared with the aid of conventional means, devices, methods and process known in the art, such as are described for example in “Remington's Pharmaceutical Sciences”, A. R. Gennaro (Editor), 17^{th} edition, Mack Publishing Company, Easton, Pa., 1985, in particular in Part 8, Chapters 76 to 93. The corresponding description is introduced herewith by way of reference and forms part of the disclosure. The amount to be administered to the patient of the respective substituted compounds according to the invention of the above-indicated general formula I may vary and is for example dependent on the patient's weight or age and also on the type of application, the indication and the severity of the disorder. Conventionally 0.001 to 100 mg/kg, preferably 0.05 to 75 mg/kg, particularly preferably 0.05 to 50 mg of at least one such compound according to the invention are applied per kg of the patient's body weight.

The pharmaceutical composition according to the invention is preferably suitable for the treatment and/or prophylaxis of one or more disorders and/or diseases selected from the group consisting of pain, preferably pain selected from the group consisting of acute pain, chronic pain, neuropathic pain, visceral pain and joint pain; hyperalgesia; allodynia; causalgia; migraine; depression; nervous affection; axonal injuries; neurodegenerative diseases, preferably selected from the group consisting of multiple sclerosis, Alzheimer's disease, Parkinson's disease and Huntington's disease; cognitive dysfunctions, preferably cognitive deficiency states, particularly preferably memory disorders; epilepsy; respiratory diseases, preferably selected from the group consisting of asthma, bronchitis and pulmonary inflammation; coughs; urinary incontinence; overactive bladder (OAB); disorders and/or injuries of the gastrointestinal tract; duodenal ulcers; gastric ulcers; irritable bowel syndrome; strokes; eye irritations; skin irritations; neurotic skin diseases; allergic skin diseases; psoriasis; vitiligo; herpes simplex; inflammations, preferably inflammations of the intestine, the eyes, the bladder, the skin or the nasal mucous membrane; diarrhoea; pruritus; osteoporosis; arthritis; osteoarthritis; rheumatic diseases; eating disorders, preferably selected from the group consisting of bulimia, cachexia, anorexia and obesity; medication dependency; misuse of medication; withdrawal symptoms in medication dependency; development of tolerance to medication, preferably to natural or synthetic opioids; drug dependency; misuse of drugs; withdrawal symptoms in drug dependency; alcohol dependency; misuse of alcohol and withdrawal symptoms in alcohol dependency; for diuresis; for antinatriuresis; for influencing the cardiovascular system; for increasing vigilance; for the treatment of wounds and/or burns; for the treatment of severed nerves; for increasing libido; for modulating movement activity; for anxiolysis; for local anaesthesia and/or for inhibiting undesirable side effects, preferably selected from the group consisting of hyperthermia, hypertension and bronchoconstriction, triggered by the administration of vanilloid receptor 1 (VR1/TRPV1 receptor) agonists, preferably selected from the group consisting of capsaicin, resiniferatoxin, olvanil, arvanil, SDZ-249665, SDZ-249482, nuvanil and capsavanil.

Particularly preferably, the pharmaceutical composition according to the invention is suitable for the treatment and/or prophylaxis of one or more disorders and/or diseases selected from the group consisting of pain, preferably of pain selected from the group consisting of acute pain, chronic pain, neuropathic pain, visceral pain and joint pain; migraine; depression; neurodegenerative diseases, preferably selected from the group consisting of multiple sclerosis, Alzheimer's disease, Parkinson's disease and Huntington's disease; cognitive dysfunctions, preferably cognitive deficiency states, particularly preferably memory disorders; inflammations, preferably inflammations of the intestine, the eyes, the bladder, the skin or the nasal mucous membrane; urinary incontinence; overactive bladder (OAB); medication dependency; misuse of medication; withdrawal symptoms in medication dependency; development of tolerance to medication, preferably development of tolerance to natural or synthetic opioids; drug dependency; misuse of drugs; withdrawal symptoms in drug dependency; alcohol dependency; misuse of alcohol and withdrawal symptoms in alcohol dependency.

Most particularly preferably, the pharmaceutical composition according to the invention is suitable for the treatment and/or prophylaxis of pain, preferably of pain selected from the group consisting of acute pain, chronic pain, neuropathic pain and visceral pain.

The present invention further relates to a substituted compound according to general formula (I) and also if appropriate to a substituted compound according to general formula (I) and one or more pharmaceutically acceptable auxiliaries for use in vanilloid receptor 1-(VR1/TRPV1) regulation, preferably for use in vanilloid receptor 1-(VR1/TRPV1) inhibition and/or vanilloid receptor 1-(VR1/TRPV1) stimulation.

The present invention therefore further relates to a substituted compound according to general formula (I) and also if appropriate to a substituted compound according to general formula (I) and one or more pharmaceutically acceptable auxiliaries for use in the prophylaxis and/or treatment of disorders and/or diseases which are mediated, at least in part, by vanilloid receptors 1.

In particular, the present invention therefore further relates to a substituted compound according to general formula (I) and also if appropriate to a substituted compound according to general formula (I) and one or more pharmaceutically acceptable auxiliaries for use in the prophylaxis and/or treatment of disorders and/or diseases selected from the group consisting of pain, preferably pain selected from the group consisting of acute pain, chronic pain, neuropathic pain, visceral pain and joint pain; hyperalgesia; allodynia; causalgia; migraine; depression; nervous affection; axonal injuries; neurodegenerative diseases, preferably selected from the group consisting of multiple sclerosis, Alzheimer's disease, Parkinson's disease and Huntington's disease; cognitive dysfunctions, preferably cognitive deficiency states, particularly preferably memory disorders; epilepsy; respiratory diseases, preferably selected from the group consisting of asthma, bronchitis and pulmonary inflammation; coughs; urinary incontinence; overactive bladder (OAB); disorders and/or injuries of the gastrointestinal tract; duodenal ulcers; gastric ulcers; irritable bowel syndrome; strokes; eye irritations; skin irritations; neurotic skin diseases; allergic skin diseases; psoriasis; vitiligo; herpes simplex; inflammations, preferably inflammations of the intestine, the eyes, the bladder, the skin or the nasal mucous membrane; diarrhoea; pruritus; osteoporosis; arthritis; osteoarthritis; rheumatic diseases; eating disorders, preferably selected from the group consisting of bulimia, cachexia, anorexia and obesity; medication dependency; misuse of medication; withdrawal symptoms in medication dependency; development of tolerance to medication, preferably to natural or synthetic opioids; drug dependency; misuse of drugs; withdrawal symptoms in drug dependency; alcohol dependency; misuse of alcohol and withdrawal symptoms in alcohol dependency; for diuresis; for antinatriuresis; for influencing the cardiovascular system; for increasing vigilance; for the treatment of wounds and/or burns; for the treatment of severed nerves; for increasing libido; for modulating movement activity; for anxiolysis; for local anaesthesia and/or for inhibiting undesirable side effects, preferably selected from the group consisting of hyperthermia, hypertension and bronchoconstriction, triggered by the administration of vanilloid receptor 1 (VR1/TRPV1 receptor) agonists, preferably selected from the group consisting of capsaicin, resiniferatoxin, olvanil, arvanil, SDZ-249665, SDZ-249482, nuvanil and capsavanil.

Most particularly preferred is a substituted compound according to general formula (I) and also if appropriate to a substituted compound according to general formula (I) and one or more pharmaceutically acceptable auxiliaries for use in the prophylaxis and/or treatment of pain, preferably of pain selected from the group consisting of acute pain, chronic pain, neuropathic pain and visceral pain.

The present invention further relates to the use of at least one compound according to general formula (I) and also if appropriate of one or more pharmaceutically acceptable auxiliaries for the preparation of a pharmaceutical composition for vanilloid receptor 1-(VR1/TRPV1) regulation, preferably for vanilloid receptor 1-(VR1/TRPV1) inhibition and/or for vanilloid receptor 1-(VR1/TRPV1) stimulation, and, further for the prophylaxis and/or treatment of disorders and/or diseases which are mediated, at least in part, by vanilloid receptors 1, such as e.g. disorders and/or diseases selected from the group consisting of pain, preferably pain selected from the group consisting of acute pain, chronic pain, neuropathic pain, visceral pain and joint pain; hyperalgesia; allodynia; causalgia; migraine; depression; nervous affection; axonal injuries; neurodegenerative diseases, preferably selected from the group consisting of multiple sclerosis, Alzheimer's disease, Parkinson's disease and Huntington's disease; cognitive dysfunctions, preferably cognitive deficiency states, particularly preferably memory disorders; epilepsy; respiratory diseases, preferably selected from the group consisting of asthma, bronchitis and pulmonary inflammation; coughs; urinary incontinence; overactive bladder (OAB); disorders and/or injuries of the gastrointestinal tract; duodenal ulcers; gastric ulcers; irritable bowel syndrome; strokes; eye irritations; skin irritations; neurotic skin diseases; allergic skin diseases; psoriasis; vitiligo; herpes simplex; inflammations, preferably inflammations of the intestine, the eyes, the bladder, the skin or the nasal mucous membrane; diarrhoea; pruritus; osteoporosis; arthritis; osteoarthritis; rheumatic diseases; eating disorders, preferably selected from the group consisting of bulimia, cachexia, anorexia and obesity; medication dependency; misuse of medication; withdrawal symptoms in medication dependency; development of tolerance to medication, preferably to natural or synthetic opioids; drug dependency; misuse of drugs; withdrawal symptoms in drug dependency; alcohol dependency; misuse of alcohol and withdrawal symptoms in alcohol dependency; for diuresis; for antinatriuresis; for influencing the cardiovascular system; for increasing vigilance; for the treatment of wounds and/or burns; for the treatment of severed nerves; for increasing libido; for modulating movement activity; for anxiolysis; for local anaesthesia and/or for inhibiting undesirable side effects, preferably selected from the group consisting of hyperthermia, hypertension and bronchoconstriction, triggered by the administration of vanilloid receptor 1 (VR1/TRPV1 receptor) agonists, preferably selected from the group consisting of capsaicin, resiniferatoxin, olvanil, arvanil, SDZ-249665, SDZ-249482, nuvanil and capsavanil.

Another aspect of the present invention is a method for vanilloid receptor 1-(VR1/TRPV1) regulation, preferably for vanilloid receptor 1-(VR1/TRPV1) inhibition and/or for vanilloid receptor 1-(VR1/TRPV1) stimulation, and, further, a method of treatment and/or prophylaxis of disorders and/or diseases, which are mediated, at least in part, by vanilloid receptors 1, in a mammal, preferably of disorders and/or diseases selected from the group consisting of pain, preferably pain selected from the group consisting of acute pain, chronic pain, neuropathic pain, visceral pain and joint pain; hyperalgesia; allodynia; causalgia; migraine; depression; nervous affection; axonal injuries; neurodegenerative diseases, preferably selected from the group consisting of multiple sclerosis, Alzheimer's disease, Parkinson's disease and Huntington's disease; cognitive dysfunctions, preferably cognitive deficiency states, particularly preferably memory disorders; epilepsy; respiratory diseases, preferably selected from the group consisting of asthma, bronchitis and pulmonary inflammation; coughs; urinary incontinence; overactive bladder (OAB); disorders and/or injuries of the gastrointestinal tract; duodenal ulcers; gastric ulcers; irritable bowel syndrome; strokes; eye irritations; skin irritations; neurotic skin diseases; allergic skin diseases; psoriasis; vitiligo; herpes simplex; inflammations, preferably inflammations of the intestine, the eyes, the bladder, the skin or the nasal mucous membrane; diarrhoea; pruritus; osteoporosis; arthritis; osteoarthritis; rheumatic diseases; eating disorders, preferably selected from the group consisting of bulimia, cachexia, anorexia and obesity; medication dependency; misuse of medication; withdrawal symptoms in medication dependency; development of tolerance to medication, preferably to natural or synthetic opioids; drug dependency; misuse of drugs; withdrawal symptoms in drug dependency; alcohol dependency; misuse of alcohol and withdrawal symptoms in alcohol dependency; for diuresis; for antinatriuresis; for influencing the cardiovascular system; for increasing vigilance; for the treatment of wounds and/or burns; for the treatment of severed nerves; for increasing libido; for modulating movement activity; for anxiolysis; for local anaesthesia and/or for inhibiting undesirable side effects, preferably selected from the group consisting of hyperthermia, hypertension and bronchoconstriction, triggered by the administration of vanilloid receptor 1 (VR1/TRPV1 receptor) agonists, preferably selected from the group consisting of capsaicin, resiniferatoxin, olvanil, arvanil, SDZ-249665, SDZ-249482, nuvanil and capsavanil, which comprises administering an effective amount of at least one compound of general formula (I) to the mammal.

The effectiveness against pain can be shown, for example, in the Bennett or Chung model (Bennett, G. J. and Xie, Y. K., A peripheral mononeuropathy in rat that produces disorders of pain sensation like those seen in man, Pain 1988, 33(1), 87-107; Kim, S. H. and Chung, J. M., An experimental model for peripheral neuropathy produced by segmental spinal nerve ligation in the rat, Pain 1992, 50(3), 355-363), by tail flick experiments (e.g. according to D'Amour and Smith (J. Pharm. Exp. Ther. 72, 74 79 (1941)) or by the formalin test (e.g. according to D. Dubuisson et al., Pain 1977, 4, 161-174).

The present invention further relates to processes for preparing inventive compounds of the above-indicated general formula (I).

All reactions which can be applied for synthesizing the compounds according to the present invention can each be carried out under the conventional conditions with which the person skilled in the art is familiar, for example with regard to pressure or the order in which the components are added. If appropriate, the person skilled in the art can determine the optimum procedure under the respective conditions by carrying out simple preliminary tests. The intermediate and end products obtained using the reactions described hereinbefore can each be purified and/or isolated, if desired and/or required, using conventional methods known to the person skilled in the art. Suitable purifying processes are for example extraction processes and chromatographic processes such as column chromatography or preparative chromatography. All of the process steps of the reaction sequences which can be applied for synthesizing the compounds according to the present invention as well as the respective purification and/or isolation of intermediate or end products, can be carried out partly or completely under an inert gas atmosphere, preferably under a nitrogen atmosphere.

The substituted compounds according to the invention can be isolated both in the form of their free bases, their free acids and also in the form of corresponding salts, in particular physiologically compatible salts, i.e. physiologically acceptable salts.

The free bases of the respective substituted compounds according to the invention can be converted into the corresponding salts, preferably physiologically compatible salts, for example by reaction with an inorganic or organic acid, preferably with hydrochloric acid, hydrobromic acid, sulphuric acid, methanesulphonic acid, p-toluenesulphonic acid, carbonic acid, formic acid, acetic acid, oxalic acid, succinic acid, tartaric acid, mandelic acid, fumaric acid, maleic acid, lactic acid, citric acid, glutamic acid, saccharic acid, monomethylsebacic acid, 5-oxoproline, hexane-1-sulphonic acid, nicotinic acid, 2, 3 or 4-aminobenzoic acid, 2,4,6-trimethylbenzoic acid, α-lipoic acid, acetyl glycine, hippuric acid, phosphoric acid and/or aspartic acid. The free bases of the respective substituted compounds of the aforementioned general formula (I) and of corresponding stereoisomers can likewise be converted into the corresponding physiologically compatible salts using the free acid or a salt of a sugar additive, such as for example saccharin, cyclamate or acesulphame.

Accordingly, the free acids of the substituted compounds according to the invention can be converted into the corresponding physiologically compatible salts by reaction with a suitable base. Examples include the alkali metal salts, alkaline earth metals salts or ammonium salts [NHₓR₄₋ₓ]⁺, in which x=0, 1, 2, 3 or 4 and R represents a branched or unbranched C₁₋₄ aliphatic residue.

The substituted compounds according to the invention and of corresponding stereoisomers can if appropriate, like the corresponding acids, the corresponding bases or salts of these compounds, also be obtained in the form of their solvates, preferably in the form of their hydrates, using conventional methods known to the person skilled in the art.

If the substituted compounds according to the invention are obtained, after preparation thereof, in the form of a mixture of their stereoisomers, preferably in the form of their racemates or other mixtures of their various enantiomers and/or diastereomers, they can be separated and if appropriate isolated using conventional processes known to the person skilled in the art. Examples include chromatographic separating processes, in particular liquid chromatography processes under normal pressure or under elevated pressure, preferably MPLC and HPLC processes, and also fractional crystallisation processes. These processes allow individual enantiomers, for example diastereomeric salts formed by means of chiral stationary phase HPLC or by means of crystallisation with chiral acids, for example (+)-tartaric acid, (−)-tartaric acid or (+)-10-camphorsulphonic acid, to be separated from one another.

The chemicals and reaction components used in the reactions and schemes described below are available commercially or in each case can be prepared by conventional methods known to the person skilled in the art.

The methods with which the person skilled in the art is familiar for carrying out the reaction steps for preparing the compounds according to the invention may be inferred from the standard works on organic chemistry such as, for example, J. March, Advanced Organic Chemistry, Wiley & Sons, 6th edition, 2007; F. A. Carey, R. J. Sundberg, Advanced Organic Chemistry, Parts A and B, Springer, 5th edition, 2007; team of authors, Compendium of Organic Synthetic Methods, Wiley & Sons. In addition, further methods and also literature references can be issued by the common databases such as, for example, the Reaxys® database of Elsevier, Amsterdam, NL or the SciFinder® database of the American Chemical Society, Washington, US.

The invention will be described hereinafter with the aid of a number of examples. This description is intended merely by way of example and does not limit the general idea of the invention.

### EXAMPLES

The indication “equivalents” (“eq.” or “eq”) means molar equivalents, “RT” or “rt” means room temperature (23±7° C.), “M” are indications of concentration in mol/l, “aq.” means aqueous, “sat.” means saturated, “sol.” means solution, “conc.” means concentrated.

Further abbreviations:

ACN acetonitrile
BH₃.SMe₂ borane-methyl sulfide complex
bipy 2,2′-bipyridine/2,2′-bipyridyl
Boc tert-butyloxycarbonyl
Boc₂O di-tert-butyl dicarbonate
brine saturated aqueous sodium chloride solution
n-BuLi n-butyllithium
t-BuOH t-butanol
CC column chromatography on silica gel
d days
DCM dichloromethane
DETA diethylentriamine
DMAP 4-dimethylaminopyridine

### DMF N,N-dimethylformamide

EDC N-(3-dimethylaminopropyl)-N′-ethylcarbodiimide
ether diethyl ether
EtOAc ethyl acetate
EtOH ethanol
h hour(s)
GC gas chromatography
H₂O water
H₂SO₄ sulfuric acid
HOBt 1-hydroxybenzotriazole
m/z mass-to-charge ratio
MeOH methanol
min minutes
MS mass spectrometry
NaH sodium hydride

### NBS N-bromosuccinimide

TEA triethylamine
NiBr₂ bipy complex of nickel(II) bromide and 2,2′-bipyridine
NiCl₂.6H₂O nickel(II) chloride hexahydride
Pd/C palladium on charcoal
Pd(PPh₃)₄ tetrakis(triphenylphosphine)palladium(0)
TBTU O-(1H-benzotriazol-1-yl)-N,N,N′,N′-tetramethyluronium tetrafluoroborate
TEA triethylamine
TFA trifluoroacetic acid
Tf2O triflic anhydride
TLC thin layer chromatography
THF tetrahydrofuran
v/v volume to volume
w/w weight in weight The yields of the compounds prepared were not optimized.

All temperatures are uncorrected.

All starting materials which are not explicitly described were either commercially available (the details of suppliers such as for example Acros, Avocado, Aldrich, Apollo, Bachem, Fluka, FluoroChem, Lancaster, Manchester Organics, MatrixScientific, Maybridge, Merck, Rovathin, Sigma, TCl, Oakwood, etc. can be found in the Symyx® Available Chemicals Database of MDL, San Ramon, US or the SciFinder® Database of the ACS, Washington D.C., US, respectively, for example) or the synthesis thereof has already been described precisely in the specialist literature (experimental guidelines can be found in the Reaxys® Database of Elsevier, Amsterdam, NL or the SciFinder® Database of the ACS, Washington D.C., US, respectively, for example) or can be prepared using the conventional methods known to the person skilled in the art.

The stationary phase used for the column chromatography was silica gel 60 (0.04-0.063 mm) from E. Merck, Darmstadt.

The mixing ratios of solvents or eluents for chromatography are specified in v/v.

All the intermediate products and exemplary compounds were analytically characterized by means of ¹H-NMR spectroscopy. In addition, mass spectrometry tests (MS, m/z for [M+H]⁺) were carried out for all the exemplary compounds and selected intermediate products.

### Synthesis of Precursor Compounds:

### Synthesis of N-((2-chloro-6-(trifluoromethyl)pyridin-3-yl)methyl)-2-(3-fluoro-4-(methylsulfonamidomethyl)phenyl)propanamide

### a) Synthesis of (2-chloro-6-(trifluoromethyl)pyridin-3-yl)methanamine

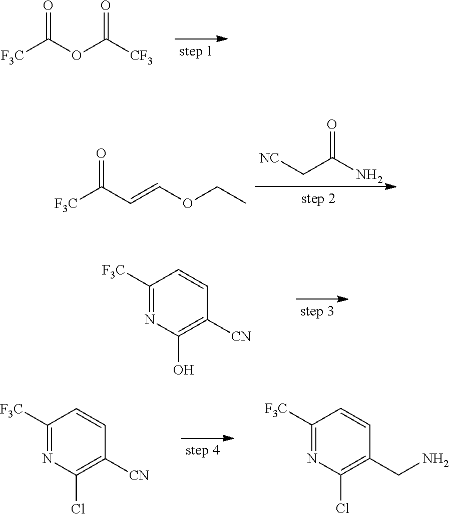

Step 1: To a stirred solution of 4-dimethylaminopyridine (734 mg, 6.8 mmol) and 2,2,2-trifluoroacetic anhydride (154.5 g, 735 mmol) in dichloromethane (600 mL), ethoxyethene (50 g, 693 mmol) was added drop wise at −10° C. The reaction mixture was stirred at 0° C. for 15-16 h and then allowed to warm at 25-30° C. TLC showed complete consumption of starting material. The organic layer was then washed with water (2×300 mL), saturated sodium bicarbonate solution (300 mL) and finally with brine (300 mL). The washed organic layer was dried over anhydrous magnesium sulfate and concentrated under atmospheric pressure to get a dark brown oily residue. This residue was finally distilled out to afford (E)-4-ethoxy-1,1,1-trifluorobut-3-en-2-one (51 g, 41%) as a colorless liquid compound.

Step 2: To a solution of 1,4-dioxane (400 mL) and cyanoacetamide (25.5 g, 0.303 mol) sodium hydride (18.2 g, 60%, 0.455 mol) was added portion wise at 10-15° C. It was allowed to stir for 30 minutes at ambient temperature after complete addition. A solution of (E)-4-ethoxy-1,1,1-trifluorobut-3-en-2-one (51 g, 0.303 mol) in 1,4-dioxane (100 mL) was added drop wise to this mixture. After complete addition the resulting solution was refluxed gently for 22 h. A solid was separated in the mixture. The mixture was cooled to room temperature and filtered through sintered funnel. The residue was washed with 100 mL of 1,4-dioxane. The washed solid was dissolved in water and acidified with 2N hydrochloric acid. The mixture was extracted with ethyl acetate (3×200 mL). The overall ethyl acetate layer was washed with water (300 mL), brine (300 mL) and finally dried over magnesium sulfate. After removal of organic solvent under reduced pressure yellow solid was afforded 2-hydroxy-6-(trifluoromethyl)nicotinonitrile (45 g, 79%).

Step 3: In a preheated mixture of 2-hydroxy-6-(trifluoromethyl)nicotinonitrile (45 g, 0.24 mol) and phosphorous oxychloride (90 mL), phosphorous pentachloride (74.6 g, 0.359 mol) was added in a small portion. A vigorous frothing came during initial addition. After complete addition the mixture was refluxed gently for 22 h. It was then checked TLC after quenching a small amount of reaction mass with saturated sodium bicarbonate solution which showed complete conversion of starting material to the product. The whole reaction mixture was then allowed to cool to 50-55° C. and excess phosphoryl chloride was removed under reduced pressure. The residue was then poured into crushed ice (−100 g) and neutralized with saturated sodium bicarbonate solution. The aqueous part was then extracted with ethyl acetate (3×200 mL). The whole organic layer was then washed with water (200 mL) and brine (200 mL) and dried over anhydrous magnesium sulfate. The removal of organic solvent under reduced pressure afforded a brown liquid compound, which was purified by column chromatography (eluent: 10% ethyl acetate in n-hexane) to afford 2-chloro-6-(trifluoromethyl)nicotinonitrile (36 g, 73%) as a light brown liquid.

Step 4: The 2-chloro-6-(trifluoromethyl)nicotinonitrile (3.5 g, 17 mmol) was dissolved in 7M isopropanolic ammonia solution (685 mL) and hydrogenated in an H-cube (10 bar, 80° C., 1.2 mL/min, 0.025 mmol/L). The removal of organic solvent under reduced pressure afforded a brownish liquid compound, which was purified by column chromatography (silica gel: 100-200 mesh, eluent: 10% ethyl acetate in methanol) to afford (2-chloro-6-(trifluoromethyl)pyridin-3-yl)methanamine (2.92 g, 84%).

### b) Synthesis of N-((2-chloro-6-(trifluoromethyl)pyridin-3-yl)methyl)-2-(3-fluoro-4-(methylsulfonamidomethyl)phenyl)propanamide

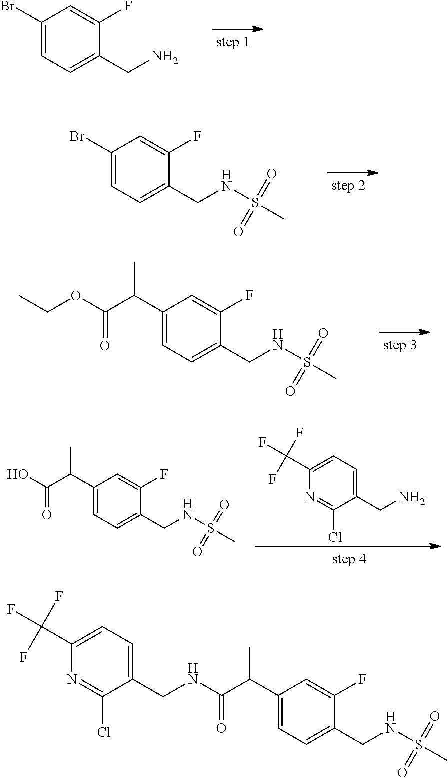

Step 1: To a stirred solution of (4-bromo-2-fluorophenyl)methanamine (25 g, 122.5 mmol) in pyridine (100 mL) at 0° C. in a protective gas atmosphere was added methanesulfonyl chloride (14.22 mL, 183.8 mmol) slowly in portions. After addition, the suspension was stirred at 0° C. for 1 h. The reaction mixture was diluted with ice cold water (20 mL) and pH was adjusted to ˜1 using 16% aqueous HCl solution. The resulting precipitation was filtered off, washed with ethyl acetate (3×20 mL) and dried overnight. The crude N-(4-bromo-2-fluorobenzyl)methanesulfonamide (29.24 g, 85%) was used as such without further purification.

Step 2: N-(4-bromo-2-fluorobenzyl)methanesulfonamide (29 g, 102.8 mmol) and ethyl-2-chloropropionate (18.26 g, 133.6 mmol) were dissolved in dimethylformamide (155 mL) in a protective gas atmosphere at room temperature. Subsequently, manganese (11.29 g, 205.6 mmol), (2,2′-bipyridine)nickel(II)dibromide (2.69 g, 7.2 mmol) and trifluoroacetic acid (1.48 mL) were added and the mixture was stirred at 65° C. for 36 h. The reaction mixture was cooled to room temperature, hydrolysed using 1 N HCl (50 mL) and extracted with diethyl ether (4×100 mL). The combined organic layer were washed with water (40 mL) and brine solution (40 mL) and dried over magnesium sulfate. The solvent was evaporated under reduced pressure and the obtained residue was purified by column chromatography (silica gel: 100-200 mesh, eluent: diethyl ether/n-hexane 9:1) to afford ethyl 2-(3-fluoro-4-(methylsulfonamidomethyl)phenyl)propanoate (12.27 g, 39%).

Step 3: The ethyl 2-(3-fluoro-4-(methylsulfonamidomethyl)phenyl)propanoate (12.5 g, 40.4 mmol) was dissolved in tetrahydrofuran-water mixture (120 mL, 2:1), lithium hydroxide (2.8 g, 121.1 mmol) was added and refluxing carried out for 12 h. After evaporation of the organic solvent under reduced pressure, the reaction mixture was extracted with diethyl ether (2×100 mL). The aqueous layer was acidified using 1 N HCl solution to pH=2 and extracted with dichloromethane (3×250 mL). The combined organic layer was dried over magnesium sulfate and concentrated under reduced pressure to afford 2-(3-fluoro-4-(methylsulfonamidomethyl)phenyl)propanoic acid (9.56 g, 86%).

Step 4: To a stirred solution of (2-chloro-6-(trifluoromethyl)pyridin-3-yl)methanamine (2.9 g, 13.8 mmol) and 2-(3-fluoro-4-(methylsulfonamidomethyl)phenyl)propanoic acid (3.8 g, 13.8 mmol) in tetrahydrofuran (100 mL) were added 1-hydroxybenzotriazol (1.89 mL, 13.8 mmol), O-(1H-benzotriazol-1-yl)-N,N,N′,N′-tetramethyluronium tetrafluoroborate (4.4 g, 13.8 mmol) and N-ethyldiisopropylamine (7 mL, 41.4 mmol) to gave an suspension. After addition of N,N-dimethylformamide (1 mL) the reaction mixture was stirred for 36 h at room temperature. The reaction mixture was concentrated under reduced pressure and the solid obtained was purified by column chromatography (eluent: cyclohexane/ethyl acetate 1:2) to afford N-((2-chloro-6-(trifluoromethyl)pyridin-3-yl)methyl)-2-(3-fluoro-4-(methylsulfonamidomethyl)phenyl)propanamide (3.96 g, 61%).

### Synthesis of the Exemplary Compounds:

The exemplary compounds 1-6, 15-16, 20, 25, 27, 29-38 and 43-47 were obtained by one of the methods disclosed before and thereafter. The exemplary compounds 7-14, 17-19, 21-24, 26, 28, 39-42 and 48-49 can be obtained by one of the methods disclosed before and thereafter. The person skilled in the art is aware which method has to be employed to obtain a particular exemplary compound.

### Detailed Synthesis of Selected Exemplary Compounds

### Synthesis of Example 1

### 2-(3-fluoro-4-(methylsulfonamidomethyl)phenyl)-N-((2-pentyl-6-(trifluoromethyl)pyridin-3-yl)methyl)propanamide

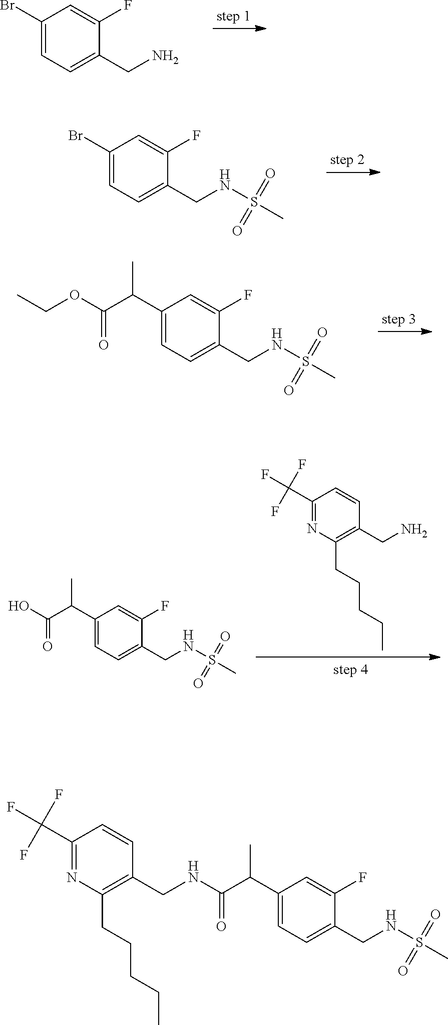 [SMILES: NCc1ccc(Br)cc1F ; CS(=O)(=O)NCc1ccc(Br)cc1F ; CCOC(=O)C(C)c1ccc(CNS(C)(=O)=O)c(F)c1 ; CC(C(=O)O)c1ccc(CNS(C)(=O)=O)c(F)c1 ; CCCCCc1nc(C(F)(F)F)ccc1CN ; CCCCCc1nc(C(F)(F)F)ccc1CNC(=O)C(C)c1ccc(CNS(C)(=O)=O)c(F)c1]

Step 1: To a stirred solution of (4-bromo-2-fluorophenyl)methanamine (5.834 g, 28.592 mmol) in pyridine were added methanesulfonyl chloride (4.2 mL, 54.325 mmol) at 0° C. The reaction mixture was stirred for 1 h, then diluted with dichloromethane. The mixture was washed with water. The organic layer was dried over magnesium sulfate and filtered. The filtrate removed in vacuo. The crude was purified by column chromatography. N-(4-bromo-2-fluorobenzyl)methanesulfonamide (7.597 g) was obtained as 93% yield.

Step 2: To a stirred solution of N-(4-bromo-2-fluorobenzyl)methanesulfonamide (2.94 g, 10.421 mmol) in dimethylformamide were added ethyl 2-chloropropionate (1.725 ml), manganese (1.145 g) and (2,2′-bipyridine)nickel(II)-dibromide (273 mg, mmol). Trifluoroacetic acid (1˜2 drops) was added. The reaction mixture was stirred for 36 h at 60° C. After cooling down to room temperature, the mixture was hydrolysed by 1 N HCl and extracted with diethyl ether. The organic layer was dried over magnesium sulfate and filtered. The filtrate removed in vacuo. The crude was purified by column chromatography. Ethyl 2-(3-fluoro-4-(methylsulfonamidomethyl)phenyl)propanoate (218 mg) was obtained.

Step 3: To a stirred solution of ethyl 2-(3-fluoro-4-(methylsulfonamidomethyl)phenyl) propanoate (458 mg, 1.51 mmol) in co-solvent with tetrahydrofuran and water (1:1) were added lithium hydroxide (190 mg, 4.529 mmol). The reaction mixture was refluxed for 15 h, then cooled to room temperature, acidified to pH 3-4 with acetic acid. The residue dissolved in ethyl acetate and washed with water and brine. The organic layer was dried over magnesium sulfate and filtered. The filtrate removed in vacuo. The crude was purified by column chromatography. 2-(3-Fluoro-4-(methylsulfonamidomethyl)phenyl)propanoic acid (218 mg) was obtained as 52% yield.

Step 4: To a stirred solution of 2-(3-fluoro-4-(methylsulfonamidomethyl)phenyl)propanoic acid (68 mg, 0.247 mmol) and (2-pentyl-6-(trifluoromethyl)pyridin-3-yl)methanamine (67 mg, 0.271 mmol) in acetonitrile were added N-(3-dimethylaminopropyl)-N′-ethylcarbodiimide (71 mg, 0.37 mmol), 1-hydroxybenzotriazole (45 mg, 0.37 mmol) and triethylamine (0.09 ml, 0.617 mmol). The reaction mixture was stirred for 15 hours at room temperature. The residue dissolved in ethyl acetate and washed with water and brine. The organic layer was dried over magnesium sulfate and filtered. The filtrate removed in vacuo. The crude was purified by column chromatography. 2-(3-Fluoro-4-(methylsulfonamidomethyl)phenyl)-N-((2-pentyl-6-(trifluoromethyl)pyridin-3-yl)methyl)propanamide (example 1) (94 mg) was obtained as 75% yield.

¹H NMR (300 MHz, CDCl₃) 7.53 (d, J=8.07 Hz, 1H), 7.43 (d, J=8.04 Hz, 1H), 7.37 (t, J=7.68 Hz, 1H), 7.09 (q, J=1.65 Hz, 1H), 7.07 (d, J=6.39 Hz, 1H), 5.70 (t, 1H), 4.68 (t, 1H), 4.48 (dd, J=3.84 Hz, 2H), 4.35 (d, J=6.42 Hz, 2H), 3.59 (q, J=7.14 Hz, 1H), 2.91 (s, 3H), 2.76 (t, 2H), 1.53 (d, J=7.14 Hz, 3H), 1.67 (m, 2H), 1.33 (m, 4H), 0.88 (m, 3H).

### Synthesis of Example 4

### (E)-N-((2-(3,3-dimethylbut-1-enyl)-6-(trifluoromethyl)pyridin-3-yl)methyl)-2-(3-fluoro-4-(methylsulfonamidomethyl)phenyl)propanamide

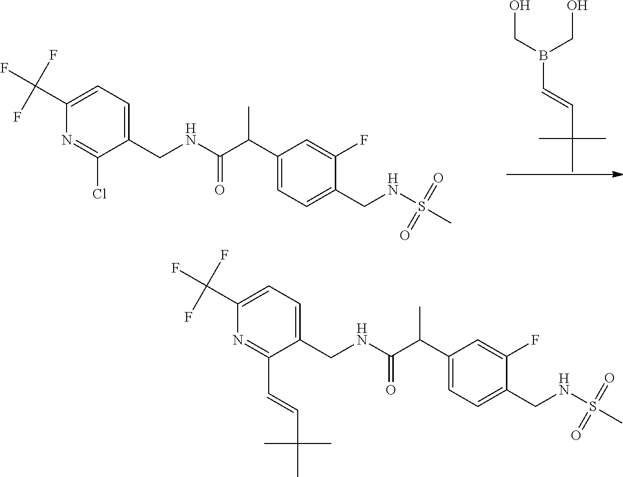 [SMILES: CC(C(=O)NCc1ccc(C(F)(F)F)nc1Cl)c1ccc(CNS(C)(=O)=O)c(F)c1 ; CC(C)(C)/C=C/B(CO)CO ; CC(C(=O)NCc1ccc(C(F)(F)F)nc1/C=C/C(C)(C)C)c1ccc(CNS(C)(=O)=O)c(F)c1]

N-((2-chloro-6-(trifluoromethyl)pyridin-3-yl)methyl)-2-(3-fluoro-4-(methylsulfonamidomethyl)-phenyl)propanamide (74 mg, 0.16 mmol) was added to a mixture of 1.0 mL toluene-ethanol (8:2). After addition of (E)-((3,3-dimethylbut-1-enyl)boranediyl)dimethanol (30 mg, 0.24 mmol), 0.152 mL 2 M aqueous sodium carbonate solution and tetrakis(triphenylphosphine)-palladium (0) (19 mg) the mixture was heated at 100° C. for 1 h in a microwave. The reaction mixture was free from oxygen by evacuating and flushing with nitrogen. After cooling to room temperature the reaction mixture was diluted with 15 mL water, extracted with ethyl acetate (2×15 mL), dried over magnesium sulfate and concentrated under reduced pressure. The solid obtained was purified by column chromatography (eluent: cyclohexane/ethyl acetate 1:2) to afford (E)-N-((2-(3,3-dimethylbut-1-enyl)-6-(trifluoromethyl)pyridin-3-yl)methyl)-2-(3-fluoro-4-(methylsulfonamidomethyl)phenyl)propanamide (example 4) (68 mg, 82%).

Examples 2, 3, 5-8, 9-14 and 44-49 were prepared in a similar manner or may be prepared analogously.

### Synthesis of Example 15

### 2-(3-fluoro-4-(methylsulfonamidomethyl)phenyl)-N-((2-isopropoxy-6-(trifluoromethyl)pyridin-3-yl)methyl)propanamide

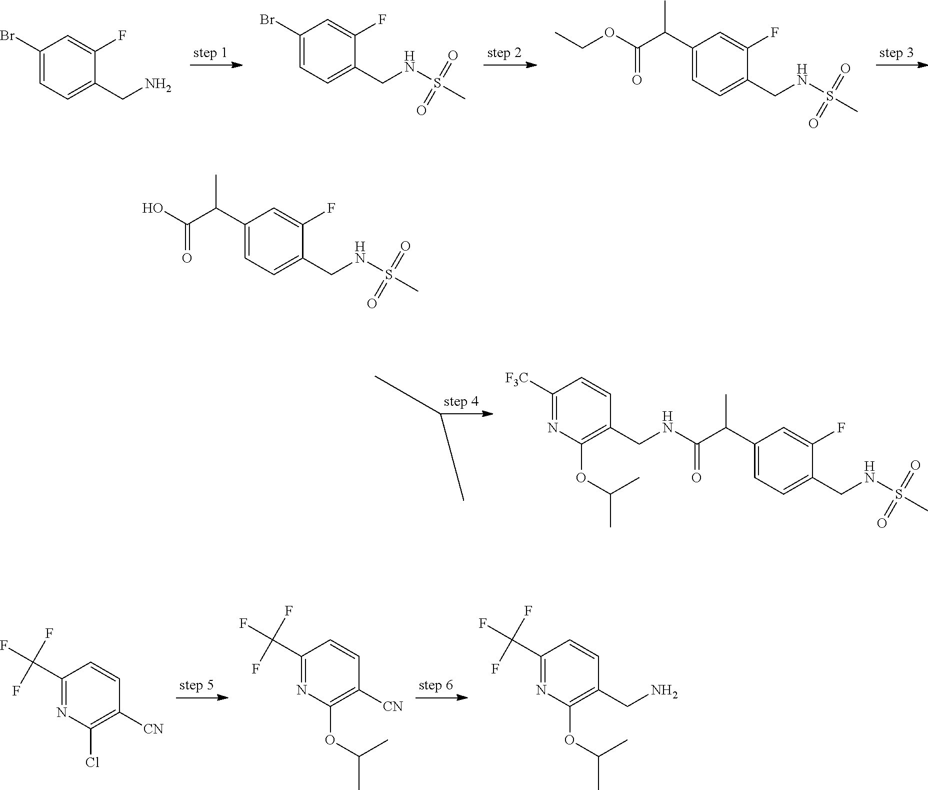

Step 1: To a stirred solution of (4-bromo-2-fluorophenyl)methanamine (5.834 g, 28.592 mmol) in pyridine were added methanesulfonyl chloride (4.2 mL, 54.325 mmol) at 0° C. The reaction mixture was stirred for 1 h, then diluted with dichloromethane. The mixture was washed with water. The organic layer was dried over magnesium sulfate and filtered. The filtrate was removed in vacuo. The crude was purified by column chromatography to give N-(4-bromo-2-fluorobenzyl)methanesulfonamide (7.597 g, 93%).

Step 2: To a stirred solution of N-(4-bromo-2-fluorobenzyl)methanesulfonamide (2.94 g, 10.421 mmol) in dimethylformamide were added ethyl 2-chloropropionate (1.725 mL), manganese (1.145 g) and (2,2′-bipyridine)nickel(II)-dibromide (273 mg, mmol). Trifluoroacetic acid (2 drops) was added. The reaction mixture was stirred for 36 h at 60° C. After cooling down to room temperature, the mixture was hydrolysed by 1 N HCl and extracted with diethyl ether. The organic layer was dried over magnesium sulfate and filtered. The filtrate was removed in vacuo. The crude was purified by column chromatography to obtain ethyl 2-(3-fluoro-4-(methylsulfonamidomethyl)phenyl)propanoate (218 mg).

Step 3: To a stirred solution of ethyl 2-(3-fluoro-4-(methylsulfonamidomethyl)phenyl)-propanoate (458 mg, 1.51 mmol) in co-solvent with tetrahydrofuran and water (1:1) were added lithium hydroxide (190 mg, 4.529 mmol). The reaction mixture was refluxed for 15 h, then cooled to room temperature, acidified to pH 3-4 with acetic acid. The residue dissolved in ethyl acetate and washed with water and brine. The organic layer was dried over magnesium sulfate and filtered. The filtrate was removed in vacuo. The crude was purified by column chromatography to give 2-(3-fluoro-4-(methylsulfonamidomethyl)phenyl)propanoic acid (218 mg, 52%).

Step 4: To a stirred solution of 2-(3-fluoro-4-(methylsulfonamidomethyl)phenyl)propanoic acid (90 mg, 0.327 mmol) (2-isopropoxy-6-(trifluoromethyl)pyridin-3-yl)methanamine (77 mg, 0.327 mmol) in acetonitrile were added N-(3-dimethylaminopropyl)-N′-ethylcarbodiimide (94 mg, 0.494 mmol), 1-hydroxybenzotriazole (66 mg, 0.491 mmol) and triethylamine (0.11 mL, 0.817 mmol). The reaction mixture was stirred for 15 h at room temperature. The residue dissolved in ethyl acetate and washed with water and brine. The organic layer was dried over magnesium sulfate and filtered. The filtrate was removed in vacuo. The crude was purified by column chromatography to obtain 2-(3-fluoro-4-(methylsulfonamidomethyl)phenyl)-N-((2-isopropoxy-6-(trifluoromethyl)pyridin-3-yl)methyl)propanamide (example 15) (86 mg, 54%).

¹H NMR (300 MHz, CDCl₃) 7.55 (d, 1H, J=7.35 Hz, Ar), 7.35 (t, 1H, J=7.78 Hz, Ar), 7.15 (d, 1H, J=7.53 Hz, Ar), 7.03 (m, 2H, Ar), 5.97 (t, 1H, NH), 5.34 (sextet, 1H), 4.71 (br, 1H, NH), 4.34 (d, 4H), 3.54 (q, 1H), 2.90 (s, 1H, mesyl), 1.49 (d, 3H), 1.28 (t, 6H)

Steps 5 and 6: analogously to steps 1-3 as described for example 25.

Examples 39, 40 and 42 may be prepared analogously.

### Synthesis of Example 16

### N-((2-butoxy-6-(trifluoromethyl)pyridin-3-yl)methyl)-2-(3-fluoro-4-(methylsulfonamidomethyl)phenyl)propanamide

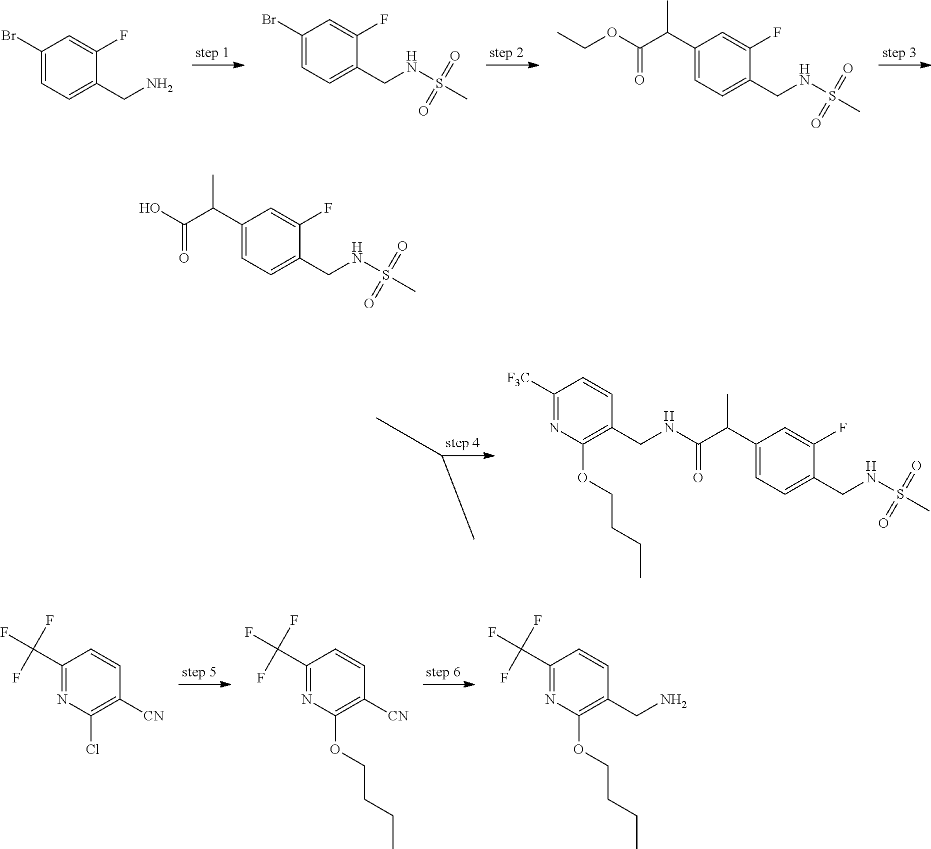

Step 1-3: according to example 15.

Step 4: To a stirred solution of 2-(3-fluoro-4-(methylsulfonamidomethyl)phenyl)propanoic acid (90 mg, 0.327 mmol) (2-butoxy-6-(trifluoromethyl)pyridin-3-yl)methanamine (81 mg, 0.327 mmol) in acetonitrile were added N-(3-dimethylaminopropyl)-N′-ethylcarbodiimide (94 mg, 0.491 mmol), 1-hydroxybenzotriazole (66 mg, 0.491 mmol) and triethylamine (0.11 mL, 0.817 mmol). The reaction mixture was stirred for 15 h at room temperature. The residue dissolved in ethyl acetate and washed with water and brine. The organic layer was dried over magnesium sulfate and filtered. The filtrate removed in vacuo. The crude was purified by column chromatography to obtain N-((2-butoxy-6-(trifluoromethyl)pyridin-3-yl)methyl)-2-(3-fluoro-4-(methylsulfonamidomethyl)phenyl)propanamide (example 16) (83 mg, 50%).

¹H NMR (300 MHz, CD₃OD) 7.48 (d, 1H, J=7.5 Hz, Ar), 7.41 (t, 1H, Ar), 7.17 (m, 3H, Ar), 4.3 (m, 6H), 3.72 (q, 1H), 2.86 (s, 3H, mesyl), 1.74 (m, 2H), 1.45 (m, 5H), 0.96 (t, 3H)

Steps 5 and 6: analogously to steps 1-3 as described for example 25.

### Synthesis of Example 20

### N-((2-(cyclopentyloxy)-6-(trifluoromethyl)pyridin-3-yl)methyl)-2-(3-fluoro-4-(methylsulfonamidomethyl)phenyl)propanamide

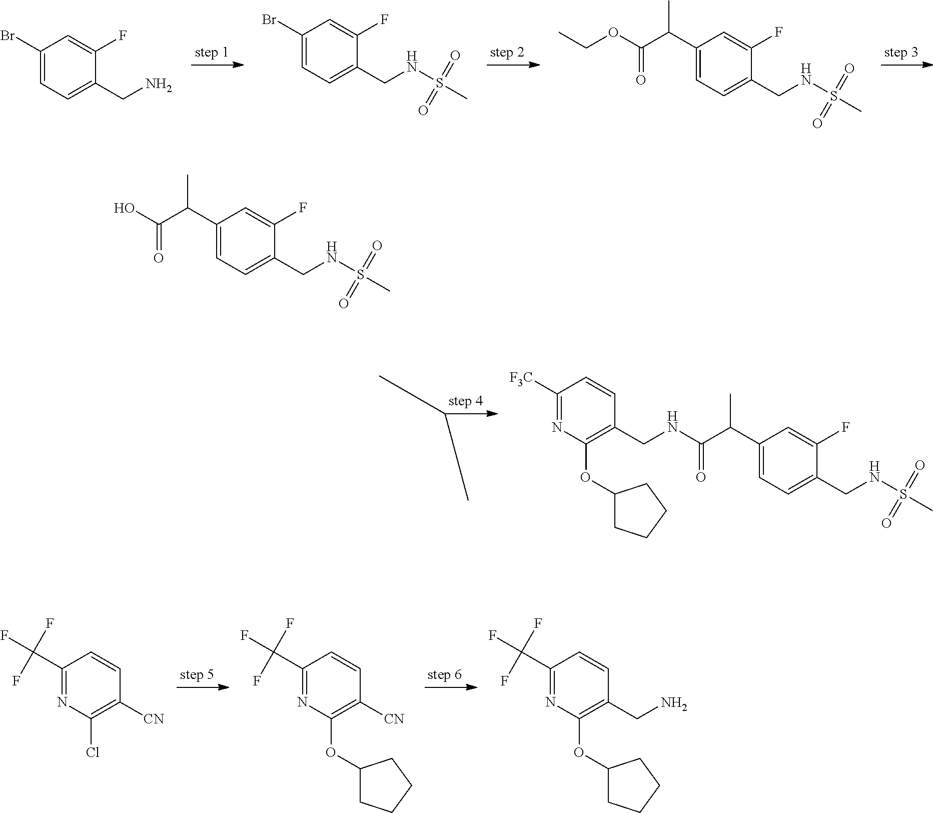

Step 1-3: according to example 15.

Step 4: To a stirred solution of 2-(3-fluoro-4-(methylsulfonamidomethyl)phenyl)propanoic acid (90 mg, 0.327 mmol) and (2-(cyclopentyloxy)-6-(trifluoromethyl)pyridin-3-yl)methanamine (81 mg, 0.327 mmol) in acetonitrile were added N-(3-dimethylaminopropyl)-N′-ethylcarbodiimide (94 mg, 0.491 mmol), 1-hydroxybenzotriazole (66 mg, 0.491 mmol) and triethylamine (0.11 mL, 0.817 mmol). The reaction mixture was stirred for 15 h at room temperature. The residue dissolved in ethyl acetate and washed with water and brine. The organic layer was dried over magnesium sulfate and filtered. The filtrate removed in vacuo. The crude was purified by column chromatography to obtain N-((2-(cyclopentyloxy)-6-(trifluoromethyl)pyridin-3-yl)methyl)-2-(3-fluoro-4-(methylsulfonamidomethyl)phenyl)-propanamide (example 20) (87 mg, 51%).

¹H NMR (300 MHz, CDCl₃) 7.53 (d, 1H, J=7.32 Hz, Ar), 7.34 (t, 1H, Ar), 7.15 (d, 1H, J=7.32 Hz, Ar), 7.03 (m, 2H, Ar), 5.91 (t, 1H, NH), 5.46 (m, 1H), 4.67 (br, 1H, NH), 4.34 (t, 4H), 3.53 (q, 1H), 2.90 (s, 1H, mesyl), 1.96 (m, 2H), 1.59 (m, 8H), 1.48 (d, 3H)

Steps 5 and 6: analogously to steps 1-3 as described for example 25.

Examples 21-24, 41 and 43 were prepared in a similar manner or may be prepared analogously.

### Synthesis of Example 25

### 2-(3-fluoro-4-(methylsulfonamidomethyl)phenyl)-N-((2-(tetrahydro-2H-pyran-4-yloxy)-6-(trifluoromethyl)pyridin-3-yl)methyl)propanamide

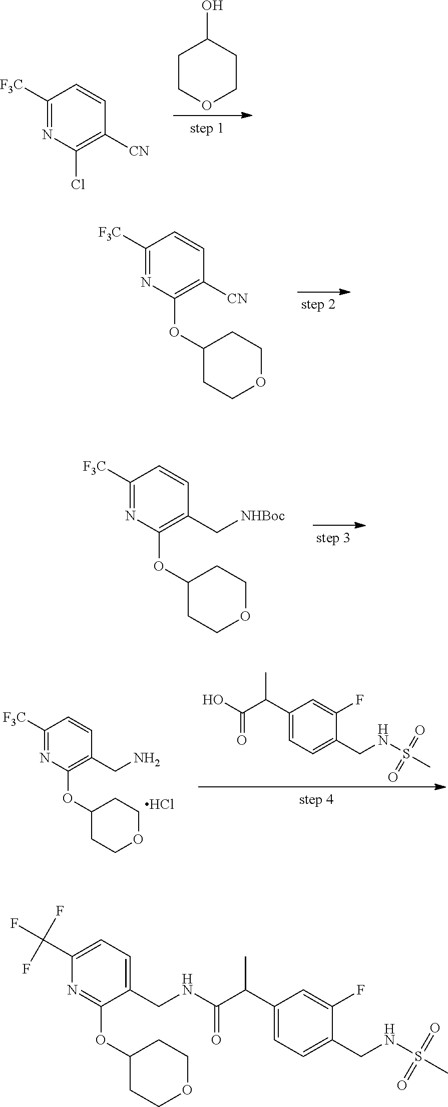

Step 1: Sodium hydride (877 mg, 22.1 mmol) was taken in a 100 ml two neck round bottom flask and washed with hexane (5 mL) under argon atmosphere. Tetrahydrofuran (5 mL) was added to the reaction mixture. Tetrahydro-4-pyranol (1.5 g, 14.7 mmol) was added to the reaction mixture drop wise at 0° C. After complete additions the reaction mixture was stirred 30 minutes at ambient temperature. Again the reaction mixture was cooled at 0° C., 2-chloro-6-(trifluoromethyl)nicotinonitrile (2.7 g, 13.2 mmol) dissolved in tetrahydrofuran (10 mL) was added to the reaction mixture drop wise. The reaction mixture was stirred 16 h at ambient temperature. TLC showed complete consumption of starting material. The reaction mixture was quenched with water. Compound was extracted with ethyl acetate (3×20 mL). The organic layer was dried over anhydrous magnesium sulfate and concentrated under reduced pressure, which was purified by column chromatography (eluent: 20% ethyl acetate in n-exane) to afford 2-(tetrahydro-2H-pyran-4-yloxy)-6-(trifluoromethyl)nicotinonitrile (2.1 g, 60%) as a white solid.

Step 2: 2-(Tetrahydro-2H-pyran-4-yloxy)-6-(trifluoromethyl)nicotinonitrile (1.5 g, 5.5 mmol) was taken in Parr hydrogenation flask in methanol (15 mL), Boc-Anhydride (1.9 mL, 8.3 mmol) was added to it. Then (10%) Pd/C (150 mg) was added to it. It was filled with 50 psi hydrogen pressure, kept for 10 h at ambient temperature. Catalyst was filtered through celite bed, filtrate was concentrated under reduced pressure to afford crude material. The crude material was purified by column chromatography (eluent: 10% ethyl acetate in n-hexane) to afford tert-butyl (2-(tetrahydro-2H-pyran-4-yloxy)-6-(trifluoromethyl)pyridin-3-yl)methylcarbamate (1.7 g, 85%) as a white solid.

Step 3: tert-Butyl (2-(tetrahydro-2H-pyran-4-yloxy)-6-(trifluoromethyl)pyridin-3-yl)methylcarbamate (1.7 g, 4.5 mmol) was dissolved in 1,4-dioxane (20 mL) and cooled it at 0° C. 1,4-dioxane hydrochloride (9 mL) was added to it. The reaction mixture was stirred 16 h at ambient temperature. TLC showed complete conversion of starting material. The reaction mixture was concentrated under reduced pressure and co-distillation with methanol. The solid compound was washed with 10% ethyl acetate in hexane (2×10 mL) and filtered to afford (2-(tetrahydro-2H-pyran-4-yloxy)-6-(trifluoromethyl)pyridin-3-yl)methanamine (1.3 g, 91%).

Step 4: To a stirred solution of 2-(3-fluoro-4-(methylsulfonamidomethyl)phenyl)propanoic acid (68 mg, 0.249 mmol) and (2-(tetrahydro-2H-pyran-4-yloxy)-6-(trifluoromethyl)pyridin-3-yl)methanamine (77 mg, 0.249 mmol) in tetrahydrofuran (2.0 mL) was added 1-hydroxybenzotriazolhydrate (0.034 mL, 0.249 mmol), O-(1H-benzotriazol-1-yl)-N,N,N′,N′-tetramethyluronium tetrafluoroborate (8 mg, 0.249 mmol) and N-ethyldiisopropylamine (0.127 mL, 0.747 mmol) to gave an suspension. After addition of N,N-dimethylformamide (0.1 mL) the reaction mixture was stirred for 40 h. The reaction mixture was concentrated under reduced pressure and the solid obtained was purified by column chromatography (silica gel: 100-200 mesh, eluent: ethyl acetate/cyclohexane 1:2) to afford 2-(3-fluoro-4-(methylsulfonamidomethyl)phenyl)-N-((2-(tetrahydro-2H-pyran-4-yloxy)-6-(trifluoromethyl)pyridin-3-yl)methyl)propanamide (example 25) (57 mg, 43%).

Examples 17-19 and 26 were prepared in a similar manner or may be prepared analogously.

### Synthesis of Example 27

### N-((2-isopropoxy-6-(trifluoromethyl)pyridin-3-yl)methyl)-2-(3-methoxy-4-(methylsulfonamidomethyl)phenyl)propanamide

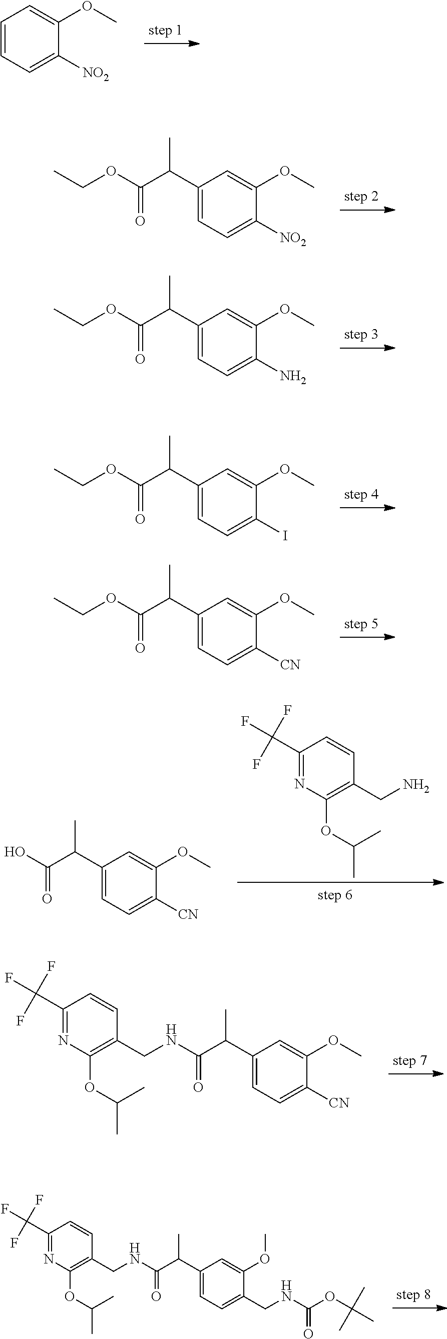

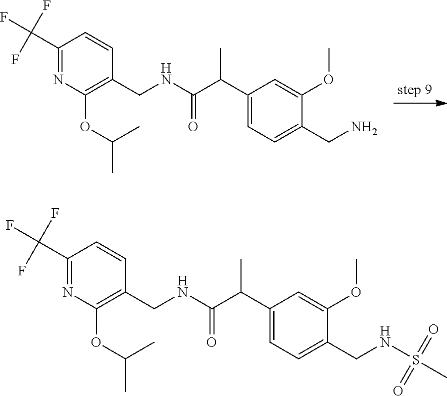 [SMILES: COc1cc(C(C)C(=O)NCc2ccc(C(F)(F)F)nc2OC(C)C)ccc1CN ; COc1cc(C(C)C(=O)NCc2ccc(C(F)(F)F)nc2OC(C)C)ccc1CNS(C)(=O)=O]

Step 1: To a stirred solution of 1-methoxy-2-nitrobenzene (3 g, 19.59 mmol) in dimethylformamide were added potassium tert-butoxide (8.792 g, 78.36 mmol) and ethyl 2-chloropropionate (2.5 ml, 19.59 mmol) while maintaining temperature below −30° C. The reaction mixture was stirred for 5 min at −30° C., then ethyl 2-chloropropionate (0.25 mL, 1.959 mmol) was added to mixture. The reaction mixture was stirred for 10 min at room temperature. The residue was dissolved in ethyl acetate and washed with water and brine. The organic layer was dried over magnesium sulfate and filtered. The filtrate removed in vacuo. The crude was purified by column chromatography. Ethyl 2-(3-methoxy-4-nitrophenyl)propanoate (683 mg) was obtained as 14% yield.

Step 2: To a stirred solution of ethyl 2-(3-methoxy-4-nitrophenyl)propanoate (683 mg, 2.697 mmol) in tetrahydrofuran and ethanol as co-solvent were added 10% Pd/C (70 mg). The mixture was charged with H₂ (gas) balloon. The resulting mixture was stirred for 15 h, then filtered using celite. The filtrate removed in vacuo. The crude was purified by column chromatography. Ethyl 2-(4-amino-3-methoxyphenyl)propanoate (447 mg) was obtained as 74% yield.

Step 3: To a stirred solution of ethyl 2-(4-amino-3-methoxyphenyl)propanoate (447 mg, 2.002 mmol) in acetonitrile and water were added p-toluenesulfonic acid monohydrate (1.142 g, 6.006 mmol), sodium nitrite (276 mg, 4.004 mmol) and potassium iodide (831 mg, 5.005 mmol). The reaction mixture was stirred for 4 h at room temperature. The mixture dissolved in ethyl acetate and washed with water and brine. The organic layer was dried over magnesium sulfate and filtered. The filtrate removed in vacuo. The crude was purified by column chromatography. Ethyl 2-(4-iodo-3-methoxyphenyl)propanoate (468 mg) was obtained as 70% yield.

Step 4: To a stirred solution of ethyl 2-(4-iodo-3-methoxyphenyl)propanoate (626 mg, 1.873 mmol) in dimethylformamide were added zinc cyanide (227 mg, 1.929 mmol) and tetrakis(triphenylphosphine) palladium (216 mg, 0.1873 mmol). The reaction mixture was stirred for 36 h at 120° C., then cooled to room temperature, diluted with ethyl acetate. The mixture was filtered using celite pad. The filtrate dissolved in ethyl acetate and extracted with NaHCO₃. The organic layer was dried over magnesium sulfate and filtered. The filtrate removed in vacuo. The crude was purified by column chromatography. Ethyl 2-(4-cyano-3-methoxyphenyl)propanoate (222 mg) was obtained as 51% yield.

Step 5: To a stirred solution of ethyl 2-(4-cyano-3-methoxyphenyl)propanoate (222 mg, 0.952 mmol) in co-solvent with tetrahydrofuran and water (1:1) were added sodium hydroxide (95 mg, 2.38 mmol). The reaction mixture was stirred for 15 h at room temperature, then acidified to pH 3-4 with acetic acid. The residue dissolved in ethyl acetate and washed with water and brine. The organic layer was dried over magnesium sulfate and filtered. The filtrate removed in vacuo. The crude was purified by column chromatography. 2-(4-Cyano-3-methoxyphenyl)propanoic acid (188 mg) was obtained as 96% yield.

Step 6: To a stirred solution of 2-(4-cyano-3-methoxyphenyl)propanoic acid (113 mg, 0.55 mmol) and (2-isopropoxy-6-(trifluoromethyl)pyridin-3-yl)methanamine (129 mg, 0.55 mmol) in acetonitrile were added N-(3-dimethylaminopropyl)-N′-ethylcarbodiimide (158 mg, 0.825 mmol), N-hydroxybenzotriazole (112 mg, 0.825 mmol) and triethylamine (0.2 mL, 1.375 mmol). The reaction mixture was stirred for 15 h at room temperature. The residue dissolved in ethyl aceate and washed with water and brine. The organic layer was dried over magnesium sulfate and filtered. The filtrate removed in vacuo. The crude was purified by column chromatography. 2-(4-Cyano-3-methoxyphenyl)-N-((2-isopropoxy-6-(trifluoromethyl)pyridin-3-yl)methyl)propanamide (187 mg) was obtained as 81% yield.

Step 7: To a stirred solution of 2-(4-cyano-3-methoxyphenyl)-N-((2-isopropoxy-6-(trifluoromethyl)pyridin-3-yl)methyl)propanamide (187 mg, 0.444 mmol) in methanol, cooled to 0° C., were added di-tert-butyl dicarbonate (194 mg, 0.888 mmol) and nickel(II) chloride hexahydride (11 mg, 0.0444 mmol). Sodium borohydride (118 mg, 3.108 mmol) was then added in small portions. The resulting reaction mixture was allowed to warm to room temperature and left to stir for 1 h. Diethylenetriamine (0.05 mL, 0.444 mmol) was added to the mixture. The mixture was stirred for 1 h. The solvent was evaporated. The residue dissolved in ethyl acetate and extracted with NaHCO₃. The organic layer was dried over magnesium sulfate and filtered. The filtrate removed in vacuo. The crude was purified by column chromatography. Tert-butyl 4-(1-((2-isopropoxy-6-(trifluoromethyl)pyridin-3-yl)methylamino)-1-oxopropan-2-yl)-2-methoxybenzylcarbamate (128 mg) was obtained as 55% yield.

Step 8: To a stirred solution of tert-butyl 4-(1-((2-isopropoxy-6-(trifluoromethyl)pyridin-3-yl)methylamino)-1-oxopropan-2-yl)-2-methoxybenzylcarbamate (128 mg, 0.244 mmol) in dichloromethane (3 mL), cooled to 0° C., were added trifluoroacetic acid (1 mL). The resulting reaction mixture was stirred for 2 h at 0° C. and 2 h at room temperature, then basified to pH 8-9 with NaHCO₃. The mixture was filtered using celite pad. The filtrate dissolved in dichloromethane and extracted with NaHCO₃. The organic layer was dried over magnesium sulfate and filtered. The filtrate removed in vacuo. The crude was purified by column chromatography. 2-(4-(Aminomethyl)-3-methoxyphenyl)-N-((2-isopropoxy-6-(trifluoromethyl)pyridin-3-yl)methyl)propanamide (108 mg) was obtained as 99% yield.

Step 9: To a stirred solution of 2-(4-(aminomethyl)-3-methoxyphenyl)-N-((2-isopropoxy-6-(trifluoromethyl)pyridin-3-yl)methyl)propanamide (108 mg, 0.253 mmol) in pyridine, cooled to 0° C., were added methanesulfonyl chloride (108 mg). The resulting reaction mixture was stirred for 15 h at room temperature. The mixture dissolved in dichloromethane and washed with 1N HCl. The organic layer was dried over magnesium sulfate and filtered. The filtrate removed in vacuo. The crude was purified by column chromatography. N-((2-isopropoxy-6-(trifluoromethyl)pyridin-3-yl)methyl)-2-(3-methoxy-4-(methylsulfonamidomethyl)phenyl)-propanamide (example 27) (57 mg) was obtained as 45% yield.

¹H-NMR (300 MHz, CDCl₃) δ 7.55 (d, J=7.5 Hz, 1H), 7.23 (d, J=7.53 Hz, 1H), 7.14 (d, J=7.32 Hz, 1H), 6.84 (d, J=7.68 Hz, 1H), 6.74 (s, 1H), 5.94 (br t, 1H), 5.30 (pentet, 1H), 4.87 (br t, 1H), 4.33 (d, J=6.24 Hz, 2H), 4.26 (d, J=6.42 Hz, 2H), 3.78 (s, 3H), 3.55 (q, J=7.14 Hz, 1H), 2.82 (s, 3H), 1.50 (d, J=7.14 Hz, 3H), 1.27-1.21 (m, 6H).

### Synthesis of Example 29

### N-((2-butoxy-6-(trifluoromethyl)pyridin-3-yl)methyl)-2-(3-methoxy-4-(methylsulfonamidomethyl)phenyl)propanamide

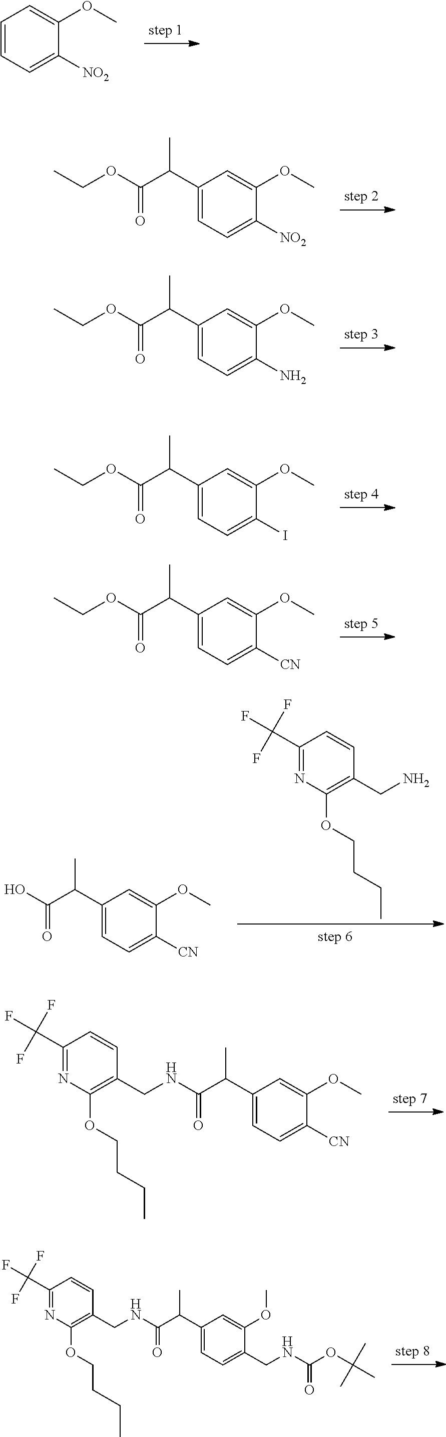

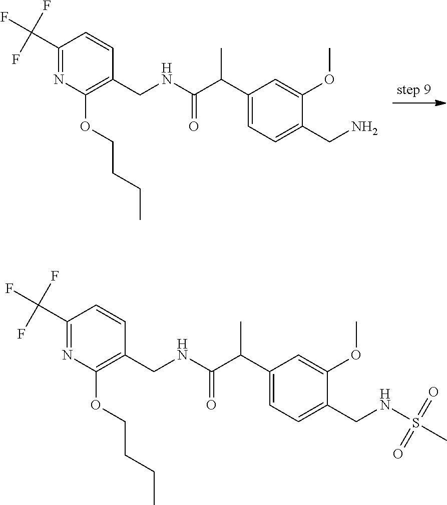 [SMILES: CCCCOc1nc(C(F)(F)F)ccc1CNC(=O)C(C)c1ccc(CN)c(OC)c1 ; CCCCOc1nc(C(F)(F)F)ccc1CNC(=O)C(C)c1ccc(CNS(C)(=O)=O)c(OC)c1]

Step 1-5: according to example 27.

Step 6: To a stirred solution of 2-(4-cyano-3-methoxyphenyl)propanoic acid (113 mg, 0.55 mmol) and (2-butoxy-6-(trifluoromethyl)pyridin-3-yl)methanamine (136 mg, 0.55 mmol) in acetonitrile were added N-(3-dimethylaminopropyl)-N′-ethylcarbodiimide (158 mg, 0.825 mmol), N-hydroxybenzotriazole (112 mg, 0.825 mmol) and triethylamine (0.2 mL, 1.375 mmol). The reaction mixture was stirred for 15 h at room temperature. The residue was dissolved in ethyl aceate and washed with water and brine. The organic layer was dried over magnesium sulfate and filtered. The filtrate removed in vacuo. The crude was purified by column chromatography. N-((2-butoxy-6-(trifluoromethyl)pyridin-3-yl)methyl)-2-(4-cyano-3-methoxyphenyl)propanamide (213 mg) was obtained as 89% yield.

Step 7: To a stirred solution of N-((2-butoxy-6-(trifluoromethyl)pyridin-3-yl)methyl)-2-(4-cyano-3-methoxyphenyl)propanamide (213 mg, 0.489 mmol) in methanol, cooled to 0° C., were added di-tert-butyl dicarbonate (213 mg, 0.978 mmol) and nickel(II) chloride hexahydride (12 mg, 0.0489 mmol). Sodium borohydride (129 mg, 3.423 mmol) was then added in small portions. The resulting reaction mixture was allowed to warm to room temperature and left to stir for 1 h. Diethylenetriamine (0.05 mL, 0.489 mmol) was added to the mixture. The mixture was stirred for 1 h. The solvent was evaporated. The residue dissolved in ethyl acetate and extracted with NaHCO₃. The organic layer was dried over magnesium sulfate and filtered. The filtrate removed in vacuo. The crude was purified by column chromatography. Tert-butyl 4-(1-((2-butoxy-6-(trifluoromethyl)pyridin-3-yl)methylamino)-1-oxopropan-2-yl)-2-methoxybenzylcarbamate (146 mg) was obtained as 55 yield.

Step 8: To a stirred solution of tert-butyl 4-(1-((2-butoxy-6-(trifluoromethyl)pyridin-3-yl)methylamino)-1-oxopropan-2-yl)-2-methoxybenzylcarbamate (146 mg, 0.271 mmol) in dichloromethane (3 mL), cooled to 0° C., were added trifluoroacetic acid (1 mL). The resulting reaction mixture was stirred for 2 h at 0° C. and 2 h at room temperature, then basified to pH 8-9 with NaHCO₃. The mixture was filtered using celite pad. The filtrate dissolved in dichloromethane and extracted with NaHCO₃. The organic layer was dried over magnesium sulfate and filtered. The filtrate removed in vacuo. The crude was purified by column chromatography. 2-(4-(Aminomethyl)-3-methoxyphenyl)-N-((2-butoxy-6-(trifluoromethyl)pyridin-3-yl)methyl)propanamide (100 mg) was obtained as 84% yield.

Step 9: To a stirred solution of 2-(4-(aminomethyl)-3-methoxyphenyl)-N-((2-butoxy-6-(trifluoromethyl)pyridin-3-yl)methyl)propanamide (100 mg, 0.227 mmol) in pyridine, cooled to 0° C., were added methanesulfonyl chloride (100 mg). The resulting reaction mixture was stirred for 15 h at room temperature. The mixture dissolved in dichloromethane and washed with 1N HCl. The organic layer was dried over magnesium sulfate and filtered. The filtrate removed in vacuo. The crude was purified by column chromatography. N-((2-butoxy-6-(trifluoromethyl)pyridin-3-yl)methyl)-2-(3-methoxy-4-(methylsulfonamidomethyl)phenyl)propanamide (example 29) (34 mg) was obtained as 29% yield.

¹H-NMR (300 MHz, CDCl₃) δ 7.55 (d, J=6.78 Hz, 1H), 7.23 (d, J=7.68 Hz, 1H), 7.17 (d, J=7.5 Hz, 1H), 6.83 (dd, J=7.5, 1.44 Hz, 1H), 6.74 (d, J=1.65 Hz, 1H), 5.93 (br t, 1H), 4.87 (br t, 1H), 4.36-4.21 (m, 6H), 3.78 (s, 3H), 3.55 (q, J=7.14 Hz, 1H), 2.82 (s, 3H), 1.69-1.62 (m, 2H), 1.49 (d, J=7.14 Hz, 3H), 1.39 (sextet, 2H), 0.95 (t, J=7.32 Hz, 3H).

Example 28 may be prepared analogously.

### Synthesis of Example 30

### 2-(4-(ethylsulfonamidomethyl)-3-fluorophenyl)-N-((2-isopropoxy-6-(trifluoromethyl)pyridin-3-yl)methyl)propanamide

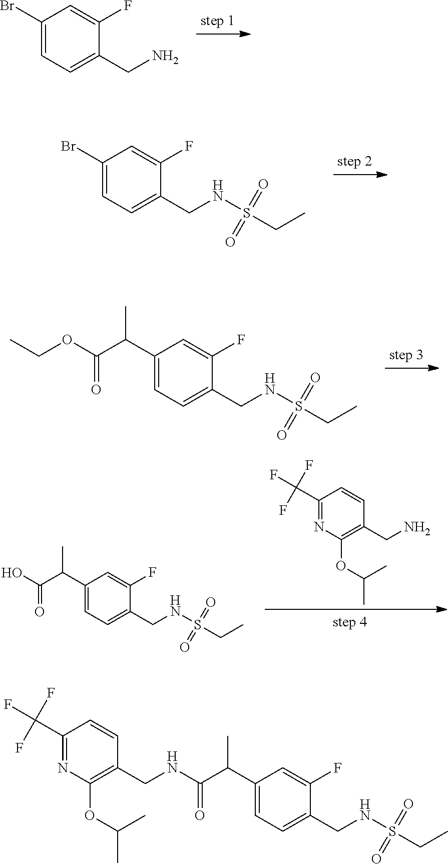

Step 1: (4-Bromo-2-fluorophenyl)methanamine (924 mg, 4.53 mmol) was dissolved in pyridine and ethane sulfonyl chloride (0.82 mL, 8.60 mmol) was added to the solution at 0° C. The mixture was stirred for 1 h at 0° C. Then, the mixture was quenched with 1N HCl and extracted with ethyl acetate. Drying over magnesium sulfate and evaporation of the ethyl acetate and purified by column chromatography gave N-(4-bromo-2-fluorobenzyl)ethane-sulfonamide in pure form (1.06 g, 79%).

Step 2: To a solution of N-(4-bromo-2-fluorobenzyl)ethanesulfonamide (305 mg, 1.03 mmol) in dimethylformamide, Manganese (113 mg, 2.06 mmol), (2,2′-bipyridine)nickel(II)-dibromide (27 mg, 0.07 mmol), ethyl 2-chloropropanoate (0.17 mL, 1.34 mmol) was added. It was followed by addition of trifluoroacetic acid (0.002 mL, 0.028 mmol). The mixture was stirred for 24 h at 65° C. The reaction mixture was quenched by concentrated HCl (7 drops). Then it was extracted with diethyl ether, dried over magnesium sulfate, the solvent was evaporated in vacuo. It was purified by column chromatography to obtain ethyl 2-(4-(ethylsulfonamidomethyl)-3-fluorophenyl)propanoate in pure form (65 mg, 20%).

Step 3: To a solution of ethyl 2-(4-(ethylsulfonamidomethyl)-3-fluorophenyl)propanoate (60 mg, 0.189 mmol) in tetrahydrofuran and water co-solvent, sodium hydroxide (19 mg) was added at room temperature. The mixture was stirred for overnight and extracted with ethyl acetate, dried over magnesium sulfate, the solvent was evaporated in vacuo. It was purified by column chromatography to give 2-(4-(ethylsulfonamidomethyl)-3-fluorophenyl)propanoic acid (55 mg).

Step 4: 2-(4-(Ethylsulfonamidomethyl)-3-fluorophenyl)propanoic acid (60 mg, 0.207 mmol) and (2-isopropoxy-6-(trifluoromethyl)pyridin-3-yl)methanamine (53 mg, 0.228 mmol) was dissolved and mixed in 1,4-dioxane, followed by addition of N-hydroxybenzotriazole (42 mg, 0.311 mmol) and N-(3-dimethylaminopropyl)-N′-ethylcarbodiimide (60 mg, 0.311 mmol) and triethylamine (0.07 mL, 0.518 mmol). The reaction mixture was stirred for overnight and then quenched by water and extracted with ethyl acetate. Drying over magnesium sulfate, evaporation of the ethyl acetate and purification by column chromatography gave 2-(4-(ethylsulfonamidomethyl)-3-fluorophenyl)-N-((2-isopropoxy-6-(trifluoromethyl)pyridin-3-yl)methyl)propanamide (example 30) (65 mg, 62%).

¹H-NMR (300 MHz, CDCl₃) δ 7.55 (d, 1H, J=6.96 Hz, Ar—H), 7.35 (t, 1H, J=7.86 Hz, Ar—H), 7.15 (d, 1H, J=7.50 Hz, Ar—H), 7.03 (m, 2H, Ar—H), 5.93 (m, 1H, amide-NH), 5.35 (m, 1H, isopropoxy-H), 4.49 (m, 1H, amide-NH), 4.33 (t, 1H, J=5.70 Hz, amide-NH), 3.55 (q, 1H, J=7.14 Hz, amide-α-H), 3.00 (q, 2H, J=7.32 Hz, ethanesulfonly-2H), 1.49 (d, 3H, J=7.14 Hz, amide-3H), 1.33 (m, 9H, ethanesulfonly-3H, isopropoxy-6H)

### Synthesis of Example 31

### N-((2-butoxy-6-(trifluoromethyl)pyridin-3-yl)methyl)-2-(4-(ethylsulfonamidomethyl)-3-fluorophenyl)propanamide

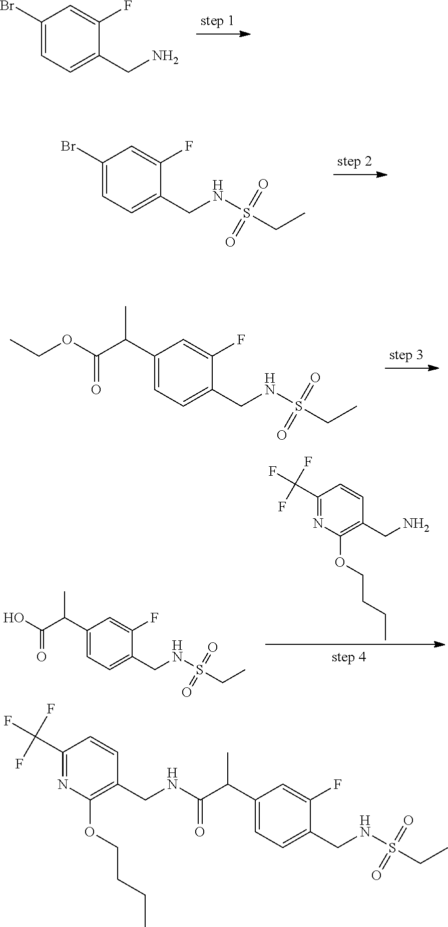

Step 1-3: according to example 30.

Step 4: 2-(4-(Ethylsulfonamidomethyl)-3-fluorophenyl)propanoic acid (60 mg, 0.207 mmol) and (2-butoxy-6-(trifluoromethyl)pyridin-3-yl)methanamine (57 mg, 0.228 mmol) was dissolved and mixed in 1,4-dioxane, followed by addition of N-hydroxybenzotriazole (42 mg, 0.311 mmol) and N-(3-dimethylaminopropyl)-N′-ethylcarbodiimide (60 mg, 0.311 mmol) and triethylamine (0.07 mL, 0.518 mmol). The reaction mixture was stirred for overnight and then quenched by water and extracted with ethyl acetate. Drying over magnesium sulfate, evaporation of the ethyl acetate and purification by column chromatography gave N-((2-butoxy-6-(trifluoromethyl)pyridin-3-yl)methyl)-2-(4-(ethylsulfonamidomethyl)-3-fluorophenyl)propanamide (example 31) (75 mg, 69%).

¹H-NMR (300 MHz, CDCl₃) δ 7.55 (d, 1H, J=7.32 Hz, Ar—H), 7.35 (t, 1H, J=7.86 Hz, Ar—H), 7.15 (d, 1H, J=7.50 Hz, Ar—H), 7.03 (m, 2H, Ar—H), 5.93 (m, 1H, amide-NH), 4.49 (m, 1H, amide-NH), 4.33 (m, 6H, Ar—CH₂, Ar—CH₂, butoxy-2H), 3.53 (q, 1H, J=7.14 Hz, amide-α-H), 3.00 (q, 2H, J=7.32 Hz, ethanesulfonly-2H), 1.70 (m, 2H, butoxy-2H), 1.48 (d, 3H, J=7.14 Hz, propionaminde-3H), 1.42 (m, 2H, butoxy 2H), 1.33 (t, 3H, J=7.32 Hz, ethanesulfonly-3H), 0.97 (t, 3H, J=7.32 Hz, butoxy 2H)

### Synthesis of Example 32

### 1-{[2-isopropoxy-6-(trifluoromethyl)pyridin-3-yl]methyl}-3-{4-[(sulfamoylamino)methyl]phenyl}urea

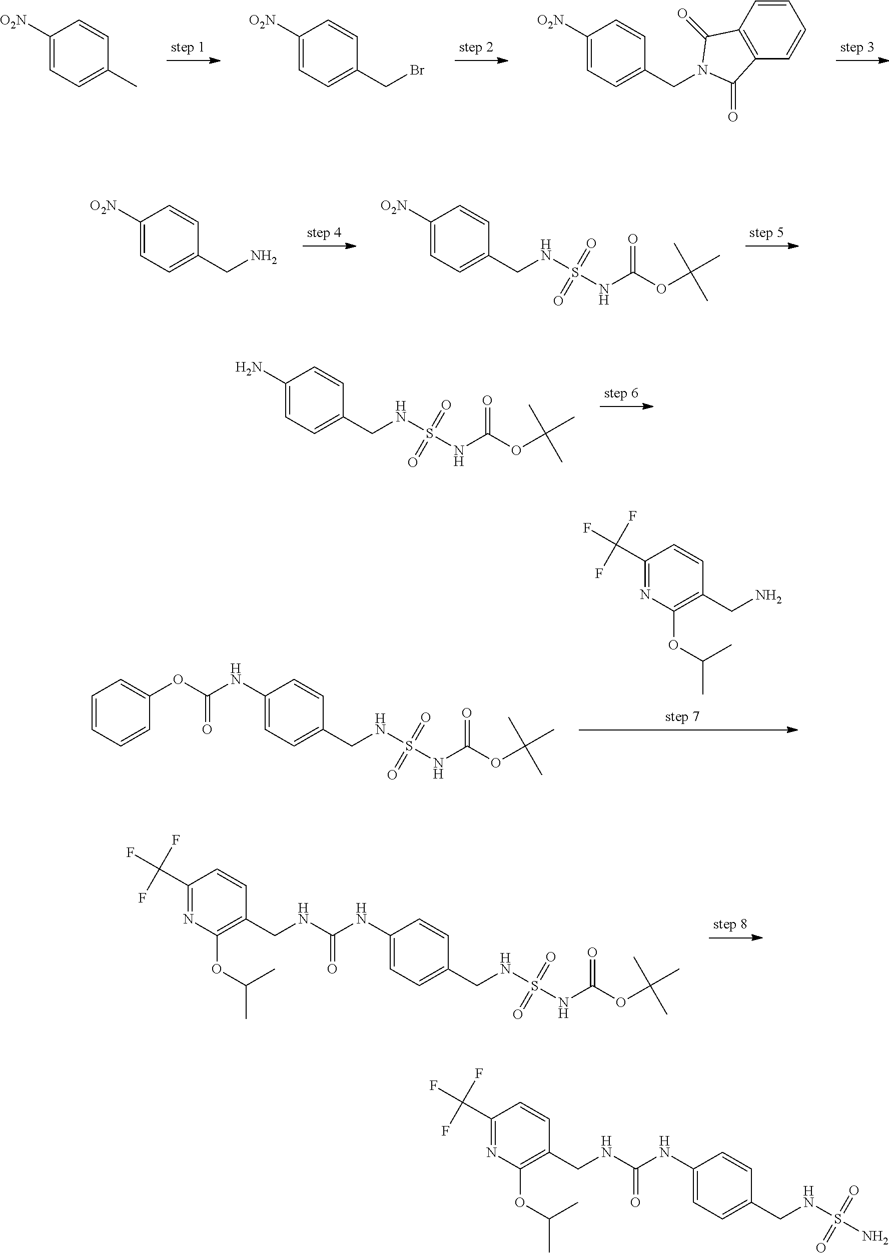

Step 1: N-Bromosuccinimide (1.51 g, 8.509 mmol) was added to a solution of 1-methyl-4-nitrobenzene (1.2 g, 7.735 mmol) in carbon tetrachloride. At room temperature 70% benzoyl peroxide (120 mg) was added to the mixture and refluxed for 24 h. The mixture was extracted with ethyl acetate, drying over magnesium sulfate, evaporation of the solvent and purification by column chromatography (silica gel: 100-200 mesh, eluent: ethyl acetate/n-hexane)1-(bromomethyl)-4-nitrobenzene (1.1 g, 61%) in pure form.

Step 2: To a solution of 1-(bromomethyl)-4-nitrobenzene (1.1 g, 4.69 mmol) in dimethylformamide, potassium phthalimide (1.9 g, 10.314 mmol) was added. The mixture was reacted for overnight, extracted with ethyl acetate and washed by brine (3×20 mL). Drying over magnesium sulfate, evaporation of the solvent and purification by column chromatography (silica gel: 100-200 mesh, eluent: ethyl acetate/n-hexane)2-(4-nitrobenzyl)isoindoline-1,3-dione (1.6 g, 99%).

Step 3: To a stirred solution of 2-(4-nitrobenzyl)isoindoline-1,3-dione (1.6 g, 5.33 mmol) in tetrahydrofuran was added hydrazine monohydrate (4 equivalents). The mixture was stirred at reflux for 6 h, until complete consumption, as evidenced by TLC analysis, the mixture was cooled to room temperature. The mixture was treated with potassium bicarbonate to adjust the pH to 12-13. It was extracted with ethyl acetate, washed by brine, dried over magnesium sulfate and concentrated under reduced pressure. The residue was purified by column chromatography (silica gel: 100-200 mesh, eluent: ethyl acetate/n-hexane) to afford (4-nitrophenyl)methanamine (592 mg, 65%).

Step 4: Chlorosulfonyl isocyanate (0.063 mL) and tert-butanol (0.07 ml) was mixed in dichloromethane (5 mL). After 10 minutes, a solution of (4-nitrophenyl)methanamine (100 mg, 0.657 mmol) in dichloromethane was added and stirred for 30 minutes at 50° C. The mixture was allowed to cool to room temperature, triethylamine (0.11 mL) was added and the mixture was stirred for 3 h more. The reaction mixture was extracted with ethyl acetate, washed by brine and dried over magnesium sulfate. After evaporation of the ethyl acetate the crude compound was purified by column chromatography (silica gel: 100-200 mesh, eluent: ethyl acetate/n-hexane) to gave tert-butyl N-(4-nitrobenzyl)sulfamoylcarbamate (112 mg, 51%).

Step 5: 10% Pd/C (7 mg) was added to a solution of tert-butyl N-(4-nitrobenzyl)sulfamoyl-carbamate (65 mg) in ethanol and tetrahydrofuran. The mixture was charged with hydrogen gas balloon and stirred for 6 h at room temperature. The mixture was filtered using celite and evaporated in vacuo to gave tert-butyl N-(4-aminobenzyl)sulfamoylcarbamate (58 mg, 98%).

Step 6: To a stirred solution of tert-butyl N-(4-aminobenzyl)sulfamoylcarbamate (86 mg, 0.285 mmol) in tetrahydrofuran-acetonitrile (1:1) was added pyridine (0.03 mL, 0.342 mmol) and phenylchloroformate (0.04 mL, 0.3 mmol) at 0° C. The mixture was stirred at 0° C. for 30 min and heated up to room temperature, then it was stirred for 30 min. The mixture was extracted with ethyl acetate, washed by brine, dried over magnesium sulfate and concentrated in vacuo. Purification by column chromatography (silica gel: 100-200 mesh, eluent: ethyl acetate/n-hexane) gave the tert-butyl N-(4-(3-((phenylcarbamate)methyl)-ureido)benzyl)sulfamoylcarbamate in pure form (59 mg, 49%).

Step 7: Tert-butyl N-(4-(3-((phenylcarbamate)methyl)ureido)benzyl)sulfamoylcarbamate (100 mg, 0.237 mmol) was dissolved in acetonitrile. (2-Isopropoxy-6-(trifluoromethyl)pyridin-3-yl)methanamine (56 mg, 0.237 mmol) and 4-dimethylaminopyridine (29 mg) were added to the solution. The reaction mixture was stirred for overnight at 50° C. The mixture was extracted with ethyl acetate and washed with brine. Drying over magnesium sulfate, evaporation of the ethyl acetate and purification by column chromatography tert-butyl N-(4-(3-((2-isopropoxy-6-(trifluoromethyl)pyridin-3-yl)methyl)ureido)benzyl)sulfamoylcarbamate (93 mg, 70%).

Step 8: To a solution tert-butyl N-(4-(3-((2-isopropoxy-6-(trifluoromethyl)pyridin-3-yl)methyl)ureido)benzyl)sulfamoylcarbamate (93 mg, 0.165 mmol) in dichloromethane (6 mL) trifluoroacetic acid (2 mL) was added at 0° C. The mixture was stirred for 30 min at 0° C. and for 2 h at room temperature. The mixture was neutralized by sodium bicarbonate to pH 7-8, extracted with ethyl acetate and washed with brine. Drying over magnesium sulfate, evaporation of the ethyl acetate and purification by column chromatography 1-{[2-isopropoxy-6-(trifluoromethyl)pyridin-3-yl]methyl}-3-{4-[(sulfamoylamino)methyl]phenyl}urea (example 32) (50 mg, 66%).

¹H-NMR (400 MHz, CD₃OD) δ 7.75 (d, 1H, J=7.44 Hz, Ar—H), 7.33 (m, 2H, Ar—H), 7.28 (m, 3H, Ar—H), 5.40 (m, 1H, isopropoxy-H), 4.35 (s, 2H), 4.12 (s, 2H), 1.38 (d, 6H, J=6.12 Hz)

### Synthesis of Example 33

### 1-{[2-butoxy-6-(trifluoromethyl)pyridin-3-yl]methyl}-3-{4-[(sulfamoylamino)methyl]phenyl}urea

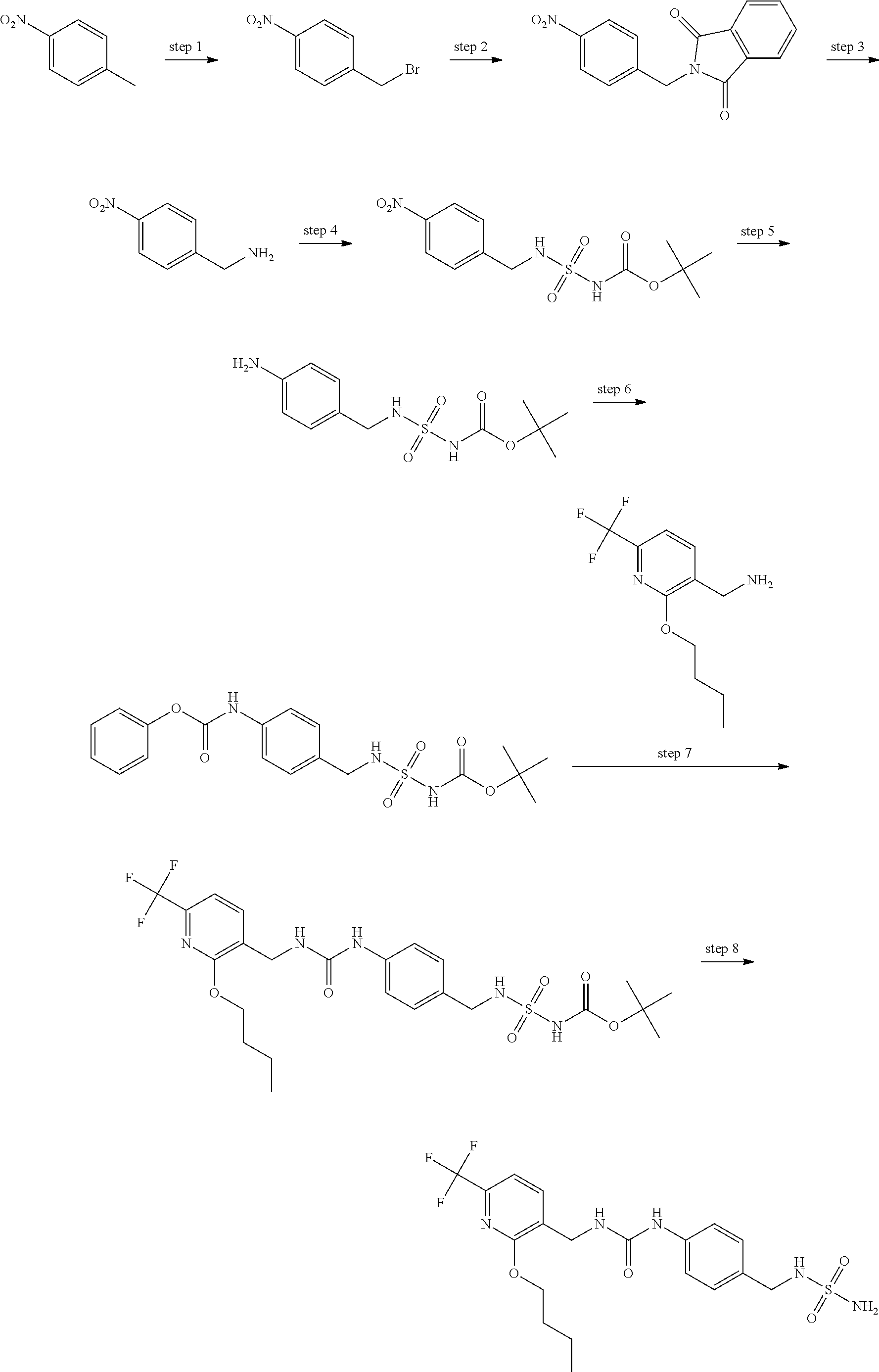

Step 1-6: according to example 32.

Step 7: Tert-butyl N-(4-(3-((phenylcarbamate)methyl)ureido)benzyl)sulfamoylcarbamate (100 mg, 0.237 mmol) was dissolved in acetonitrile. (2-Butoxy-6-(trifluoromethyl)pyridin-3-yl)methanamine (59 mg, 0.237 mmol) and 4-dimethylaminopyridine (29 mg) were added to the solution. The reaction mixture was stirred for overnight at 50° C. The mixture was extracted with ethyl acetate and washed with brine. Drying over magnesium sulfate, evaporation of the ethyl acetate and purification by column tert-butyl N-(4-(3-((2-butoxy-6-(trifluoromethyl)pyridin-3-yl)methyl)ureido)benzyl)sulfamoylcarbamate (85 mg, 63%).

Step 8: To a tert-butyl N-(4-(3-((2-butoxy-6-(trifluoromethyl)pyridin-3-yl)methyl)ureido)benzyl)sulfamoylcarbamate (85 mg, 0.148 mmol) in dichloromethane (6 mL) trifluoroacetic acid (2 mL) was added at 0° C. The mixture was stirred for 30 min at 0° C. and for 2 h at room temperature. The mixture was neutralized by sodium bicarbonate to pH 7-8, extracted with ethyl acetate and washed with brine. Drying over magnesium sulfate, evaporation of the ethyl acetate and purification by column chromatography 1-{[2-butoxy-6-(trifluoromethyl)pyridin-3-yl]methyl}-3-{4-[(sulfamoylamino)methyl]phenyl}urea (60 mg, 85%).

¹H-NMR (300 MHz, CD₃OD) δ 7.76 (d, 2H, J=7.32 Hz, Ar—H), 7.33 (m, 5H, Ar—H), 4.43 (m, 4H, butoxy-2H and Ar—CH₂), 4.13 (s, 2H, Ar—CH₂), 1.85 (m, 2H, butoxy-2H), 1.54 (m, 2H, butoxy-2H), 1.01 (t, 3H, J=7.50 Hz, butoxy-3H)

### Synthesis of Example 34

### 1-{[2-cyclopentyloxy-6-(trifluoromethyl)pyridin-3-yl]methyl}-3-{4-[(sulfamoylamino)methyl]phenyl}urea

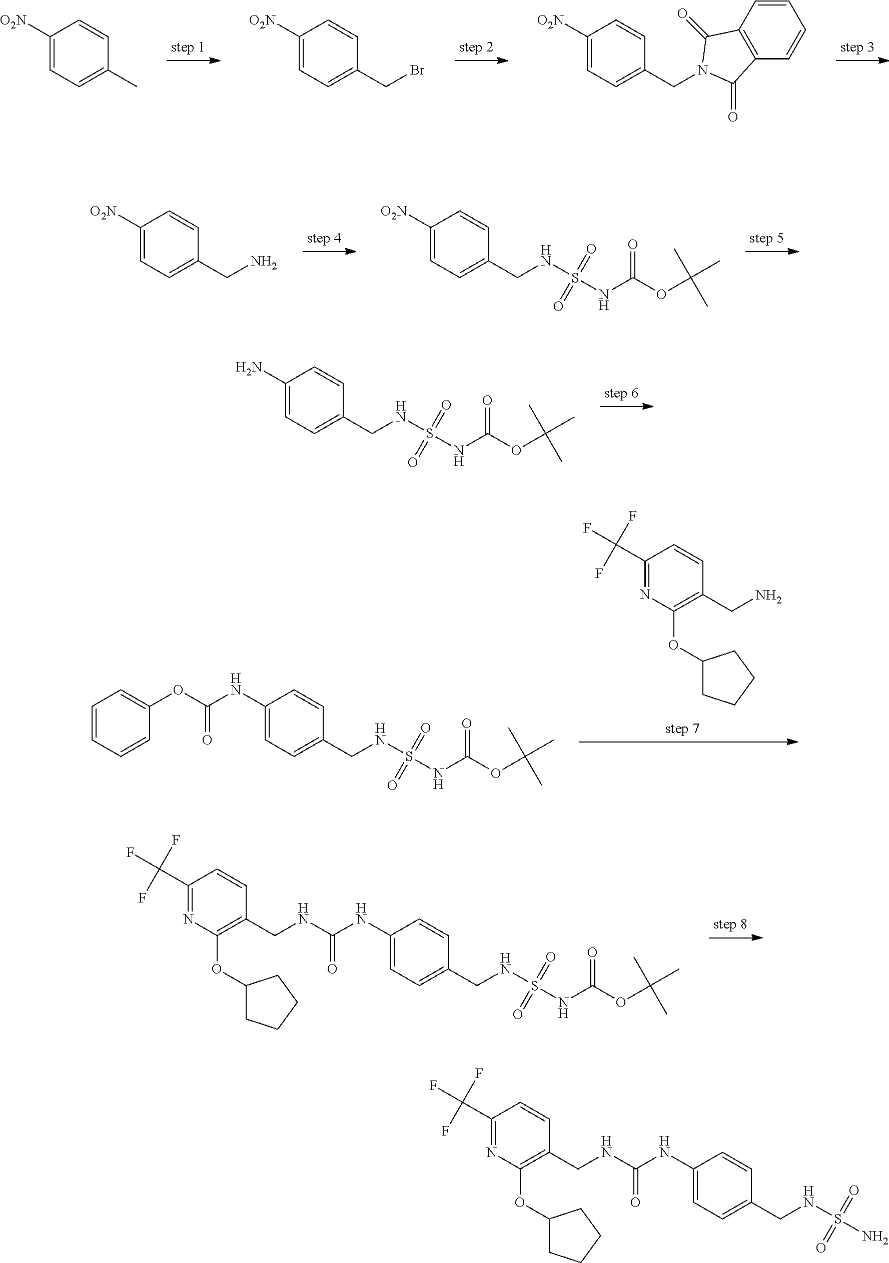

Step 1-6: according to example 32.

Step 7: Tert-butyl N-(4-(3-((phenylcarbamate)methyl)ureido)benzyl)sulfamoylcarbamate (100 mg, 0.237 mmol) was dissolved in acetonitrile. (2-(Cyclopentyloxy)-6-(trifluoromethyl)-pyridin-3-yl)methanamine (62 mg, 0.237 mmol) and 4-dimethylaminopyridine (29 mg) were added to the solution. The reaction mixture was stirred for overnight at 50° C. The mixture was extracted with ethyl acetate and washed with brine. Drying over magnesium sulfate, evaporation of the ethyl acetate and purification by column tert-butyl N-(4-(3-((2-(cyclopentyloxy)-6-(trifluoromethyl)pyridin-3-yl)methyl)ureido)benzyl)sulfamoylcarbamate (97 mg, 70%).

Step 8: To a tert-butyl N-(4-(3-((2-(cyclopentyloxy)-6-(trifluoromethyl)pyridin-3-yl)methyl)ureido)benzyl)sulfamoylcarbamate (97 mg, 0.165 mmol) in dichloromethane (6 mL) trifluoroacetic acid (2 mL) was added at 0° C. The mixture was stirred for 30 min at 0° C. and for 2 h at room temperature. The mixture was neutralized by sodium bicarbonate to pH 7-8, extracted with ethyl acetate and washed with brine. Drying over magnesium sulfate, evaporation of the ethyl acetate and purification by column chromatography 1-{[2-cyclopentyloxy-6-(trifluoromethyl)pyridin-3-yl]methyl}-3-{4-[(sulfamoylamino)-methyl]phenyl}urea (example 33) (60 mg, 75%).

¹H-NMR (400 MHz, CD₃OD) δ 7.75 (d, 1H, J=7.44 Hz, Ar—H), 7.33 (m, 2H, Ar), 7.27 (m, 3H, Ar) 5.51 (m, 1H, penthoxy-1H), 4.34 (s, 2H, Ar—CH₂), 4.12 (s, 2H, Ar—CH₂), 2.02 (m, 2H, penthoxy-2H), 1.82 (m, 4H, penthoxy-4H), 1.66 (m, 2H, penthoxy-2H)

### Synthesis of Example 35

### 1-{3-fluoro-4-[(sulfamoylamino)methyl]phenyl}-3-{[2-ethoxy-6-(trifluoromethyl)pyridin-3-yl]methyl}urea

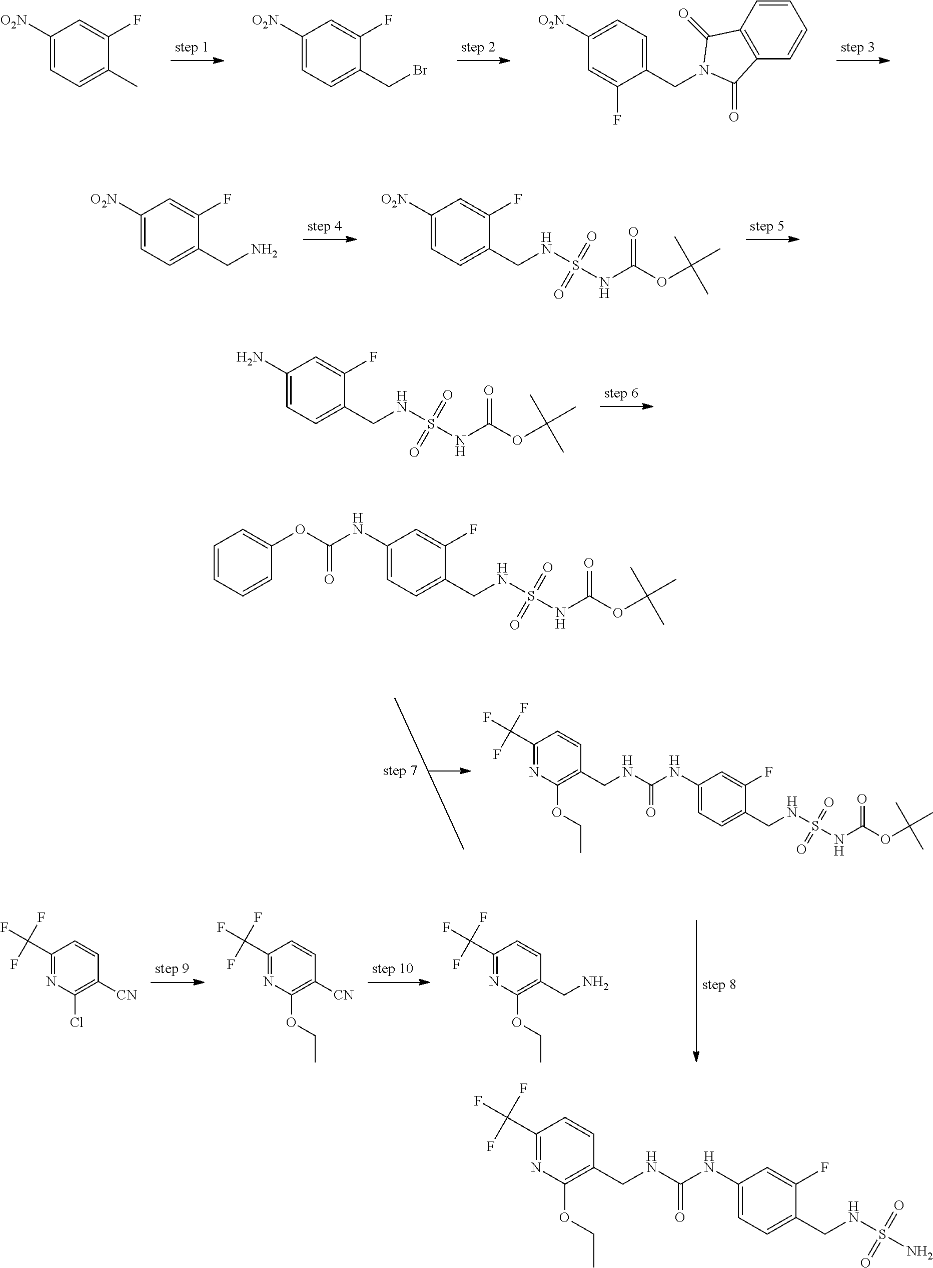

Step 1: N-Bromosuccinimide (1.27 g, 7.09 mmol) was added to a solution of 2-fluoro-1-methyl-4-nitrobenzene (1.0 g, 6.446 mmol) in carbon tetrachloride. At room temperature 70% benzoyl peroxide (150 mg) was added to the mixture and refluxed for 24 h. The mixture was extracted with ethyl acetate, drying over magnesium sulfate, evaporation of the solvent and purification by column chromatography (silica gel: 100-200 mesh, eluent: ethyl acetate/n-hexane)1-(bromomethyl)-2-fluoro-4-nitrobenzene (1.05 g, 69%).

Step 2: To a solution of 1-(bromomethyl)-2-fluoro-4-nitrobenzene (1.05 g, 4.48 mmol) in dimethylformamide, potassium phthalimide (1.8 g, 9.852 mmol) was added. The mixture was reacted for overnight, extracted with ethyl acetate and washed by brine (3×20 mL). Drying over magnesium sulfate, evaporation of the solvent and purification by column chromatography (silica gel: 100-200 mesh, eluent: ethyl acetate/n-hexane)2-(2-fluoro-4-nitrobenzyl)isoindoline-1,3-dione (1.35 g, 99%).

Step 3: To a stirred solution of 2-(2-fluoro-4-nitrobenzyl)isoindoline-1,3-dione (1.35 g, 4.48 mmol) in tetrahydrofuran was added hydrazine monohydrate (1.3 mL). The mixture was stirred at reflux for 6 h, until complete consumption, as evidenced by TLC analysis, the mixture was cooled to room temperature. The mixture was treated with potassium bicarbonate to adjust the pH to 12-13. It was extracted with ethyl acetate, washed by brine, dried over magnesium sulfate and concentrated under reduced pressure. The residue was purified by column chromatography (silica gel: 100-200 mesh, eluent: ethyl acetate/n-hexane) to afford (2-fluoro-4-nitrophenyl)methanamine (316 mg, 41%).

Step 4: Chlorosulfonyl isocyanate (0.1 mL) and tert-butanol (0.12 ml) was mixed in dichloromethane (5 mL). After 10 minutes, a solution of (2-fluoro-4-nitrophenyl)methanamine (200 mg, 1.176 mmol) in dichloromethane was added and stirred for 30 minutes at 50° C. The mixture was allowed to cool to room temperature, triethylamine (0.11 mL) was added and the mixture was stirred for 3 h more. The reaction mixture was extracted with ethyl acetate, washed by brine and dried over magnesium sulfate. After evaporation of the ethyl acetate the crude compound was purified by column chromatography (silica gel: 100-200 mesh, eluent: ethyl acetate/n-hexane) to gave tert-butyl N-(2-fluoro-4-nitrobenzyl)sulfamoylcarbamate (139 mg, 34%).

Step 5: 10% Pd/C (42 mg) was added to a solution of tert-butyl N-(2-fluoro-4-nitrobenzyl)sulfamoylcarbamate (135 mg) in ethanol and tetrahydrofuran. The mixture was charged with hydrogen gas balloon and stirred for 6 h at room temperature. The mixture was filtered using celite and evaporated in vacuo to tert-butyl N-(4-amino-2-fluorobenzyl)sulfamoylcarbamate (127 mg, 99%).

Step 6: To a stirred solution of tert-butyl N-(4-amino-2-fluorobenzyl)sulfamoylcarbamate (127 mg, 0.398 mmol) in tetrahydrofuran-acetonitrile (1:1) was added pyridine (0.04 mL, 0.478 mmol) and phenylchloroformate (0.05 mL, 0.418 mmol) at 0° C. The mixture was stirred at 0° C. for 30 min and heated up to room temperature, then it was stirred for 30 min. The mixture was extracted with ethyl acetate, washed by brine, dried over magnesium sulfate and concentrated in vacuo. Purification by column chromatography (silica gel: 100-200 mesh, eluent: ethyl acetate/n-hexane) gave the tert-butyl N-(2-fluoro-4-(phenylcarbamate)methyl)ureido)benzyl)sulfamoylcarbamate in pure form (160 mg, 91%).

Step 7: Tert-butyl N-(2-fluoro-4-(phenylcarbamate)methyl)ureido)benzyl)sulfamoylcarbamate (100 mg, 0.228 mmol) was dissolved in acetonitrile. (2-Ethoxy-6-(trifluoromethyl)pyridin-3-yl)methanamine (50 mg, 0.228 mmol) and 4-dimethylaminopyridine (17 mg) were added to the solution. The reaction mixture was stirred for overnight at 50° C. The mixture was extracted with ethyl acetate and washed with brine. Drying over magnesium sulfate, evaporation of the ethyl acetate and purification by column chromatography gave tert-butyl N-(4-(3-((2-ethoxy-6-(trifluoromethyl)pyridin-3-yl)methyl)ureido)-2-fluorobenzyl)sulfamoylcarbamate (70 mg, 54%).

Step 8: To a solution tert-butyl N-(4-(3-((2-ethoxy-6-(trifluoromethyl)pyridin-3-yl)methyl)ureido)-2-fluorobenzyl)sulfamoylcarbamate (70 mg, 0.097 mmol) in dichloromethane (6 mL) was added trifluoroacetic acid (2 mL) at 0° C. The mixture was stirred for 30 min at 0° C. and for 2 h at room temperature. The mixture was neutralized by sodium bicarbonate to pH 7-8, extracted with ethyl acetate and washed with brine. Drying over magnesium sulfate, evaporation of the ethyl acetate and purification by column chromatography gave 1-{3-fluoro-4-[(sulfamoylamino)methyl]phenyl}-3-{[2-ethoxy-6-(trifluoromethyl)pyridin-3-yl]methyl}urea (example 35) (35 mg, 60%).

¹H-NMR (300 MHz, CD₃OD) δ 7.76 (d, 1H, J=7.68 Hz, Ar—H), 7.38 (m, 3H, Ar—H), 7.01 (dd, 1H, J₁=8.25 Hz, J₂=2.04 Hz Ar—H), 4.47 (q, 2H, J=7.14 Hz, ethoxy-2H), 4.44 (s, 2H, Ar—CH₂), 4.17 (s, 2H, Ar—CH₂), 1.44 (t, 3H, J=7.14 Hz, ethoxy-3H)

Steps 9 and 10: analogously to steps 1-3 as described for example 25.

### Synthesis of Example 36

### 1-{3-fluoro-4-[(sulfamoylamino)methyl]phenyl}-3-{[2-isopropoxy-6-(trifluoromethyl)pyridin-3-yl]methyl}urea

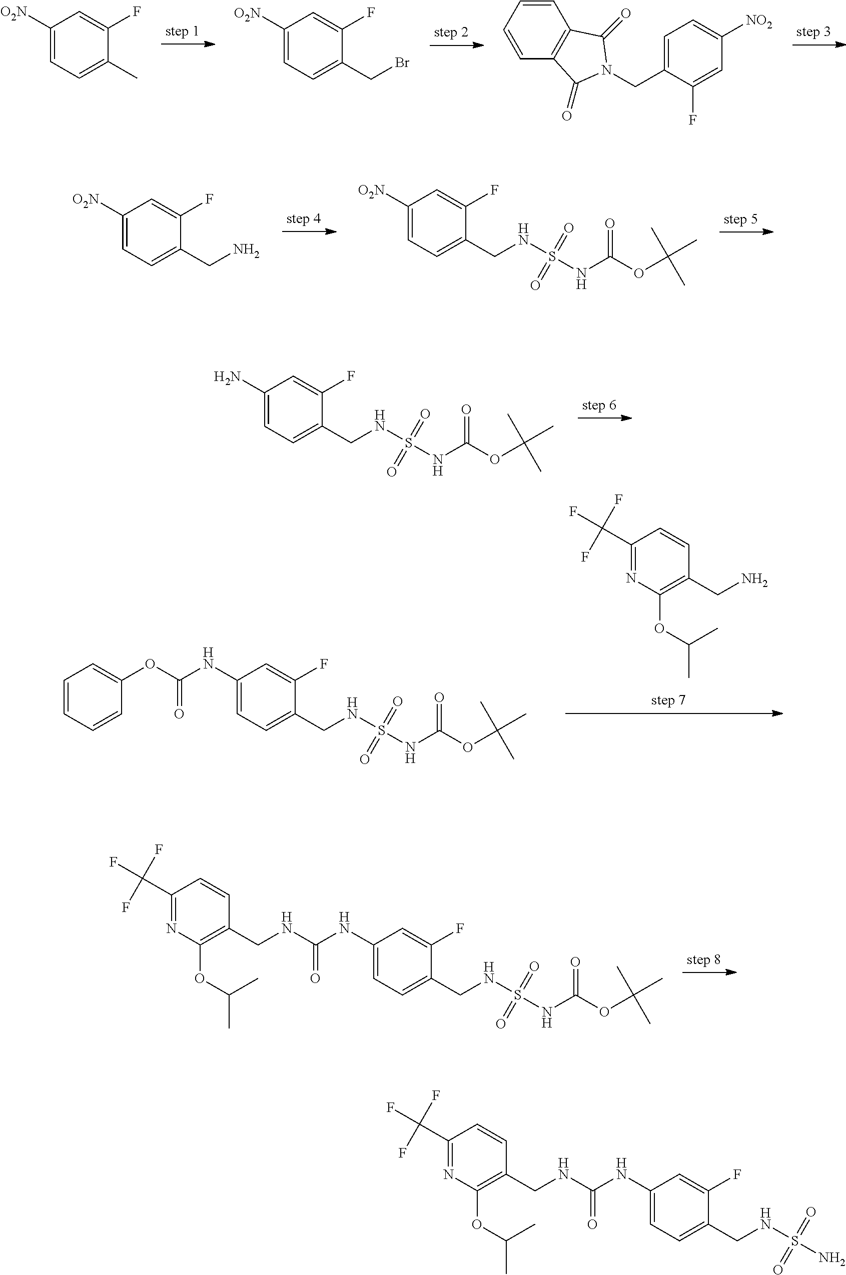

Step 1-6: according to example 35.

Step 7: Tert-butyl N-(2-fluoro-4-(phenylcarbamate)methyl)ureido)benzyl)sulfamoylcarbamate (100 mg, 0.228 mmol) was dissolved in acetonitrile. (2-Isopropoxy-6-(trifluoromethyl)pyridin-3-yl)methanamine (53 mg, 0.228 mmol) and 4-dimethylaminopyridine (17 mg) were added to the solution. The reaction mixture was stirred for overnight at 50° C. The mixture was extracted with ethyl acetate and washed with brine. Drying over magnesium sulfate, evaporation of the ethyl acetate and purification by column chromatography gave tert-butyl N-(2-fluoro-4-(3-((2-isopropoxy-6-(trifluoromethyl)pyridin-3-yl)methyl)ureido)benzyl)-sulfamoylcarbamate (70 mg, 53%).

Step 8: To a tert-butyl N-(2-fluoro-4-(3-((2-isopropoxy-6-(trifluoromethyl)pyridin-3-yl)methyl)ureido)benzyl)sulfamoylcarbamate (45 mg, 0.121 mmol) in dichloromethane (6 mL) was added trifluoroacetic acid (2 mL) at 0° C. The mixture was stirred for 30 min at 0° C. and for 2 h at room temperature. The mixture was neutralized by sodium bicarbonate to pH 7-8, extracted with ethyl acetate and washed with brine. Drying over magnesium sulfate, evaporation of the ethyl acetate and purification by column chromatography gave 1-{3-fluoro-4-[(sulfamoylamino)methyl]phenyl}-3-{[2-isopropoxy-6-(trifluoromethyl)pyridin-3-yl]methyl}urea (example 36) (45 mg, 78%).

¹H-NMR (300 MHz, CD₃OD) δ 7.75 (d, 2H, J=7.50 Hz, Ar—H), 7.38 (m, 3H, Ar), 7.00 (dd, 1H, J₁=8.40 Hz, J₂=1.83 Hz, Ar—H), 5.51 (m, 1H, isopropoxy-1H), 4.35 (s, 2H, Ar—CH₂), 4.17 (s, 2H, Ar—CH₂), 1.39 (d, 6H, J=6.24 Hz, isopropoxy-6H)

### Synthesis of Example 37

### 1-{3-fluoro-4-[(sulfamoylamino)methyl]phenyl}-3-{[2-butoxy-6-(trifluoromethyl)pyridin-3-yl]methyl}urea

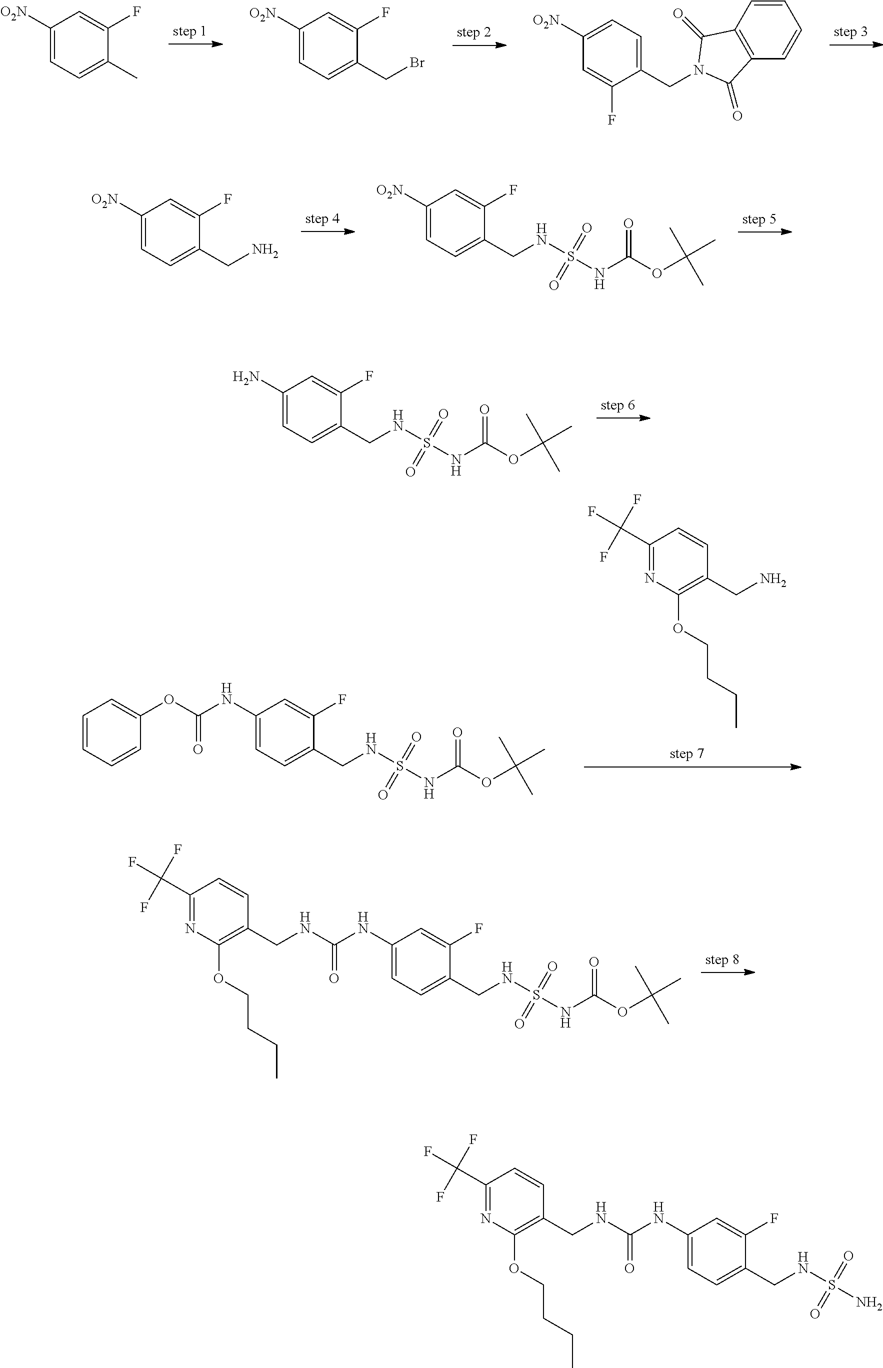

Step 1-6: according to example 35.

Step 7: Tert-butyl N-(2-fluoro-4-(phenylcarbamate)methyl)ureido)benzyl)sulfamoylcarbamate (100 mg, 0.228 mmol) was dissolved in acetonitrile. (2-Butoxy-6-(trifluoromethyl)pyridin-3-yl)methanamine (53 mg, 0.228 mmol) and 4-dimethylaminopyridine (28 mg) were added to the solution. The reaction mixture was stirred for overnight at 50° C. The mixture was extracted with ethyl acetate and washed with brine. Drying over magnesium sulfate, evaporation of the ethyl acetate and purification by column chromatography gave tert-butyl N-(4-(3-((2-butoxy-6-(trifluoromethyl)pyridin-3-yl)methyl)ureido)-2-fluorobenzyl)-sulfamoylcarbamate (85 mg, 63%).

Step 8: To a tert-butyl N-(4-(3-((2-butoxy-6-(trifluoromethyl)pyridin-3-yl)methyl)ureido)-2-fluorobenzyl)sulfamoylcarbamate (85 mg, 0.121 mmol) in dichloromethane (6 mL) was added trifluoroacetic acid (2 mL) at 0° C. The mixture was stirred for 30 min at 0° C. and for 2 h at room temperature. The mixture was neutralized by sodium bicarbonate to pH 7-8, extracted with ethyl acetate and washed with brine. Drying over magnesium sulfate, evaporation of the ethyl acetate and purification by column chromatography gave 1-{3-fluoro-4-[(sulfamoylamino)methyl]phenyl}-3-{[2-butoxy-6-(trifluoromethyl)pyridin-3-yl]methyl}urea (example 37) (60 mg, 85%).

¹H-NMR (300 MHz, CD₃OD) δ 7.76 (d, 2H, J=7.50 Hz, Ar—H), 7.38 (m, 3H, Ar), 7.01 (dd, 2H, Ar, 1H, J₁=8.25 Hz, J₂=2.01 Hz, Ar—H), 4.42 (m, 4H, butoxy-2H and Ar—CH₂), 4.17 (s, 2H, Ar—CH₂), 1.85 (m, 2H, butoxy-2H), 1.54 (m, 2H, butoxy-2H), 1.01 (t, 3H, J=7.32 Hz, butoxy-3H)

### Synthesis of Example 38

### 1-{3-fluoro-4-[(sulfamoylamino)methyl]phenyl}-3-{[2-cyclopentyloxy-6-(trifluoromethyl)pyridin-3-yl]methyl}urea

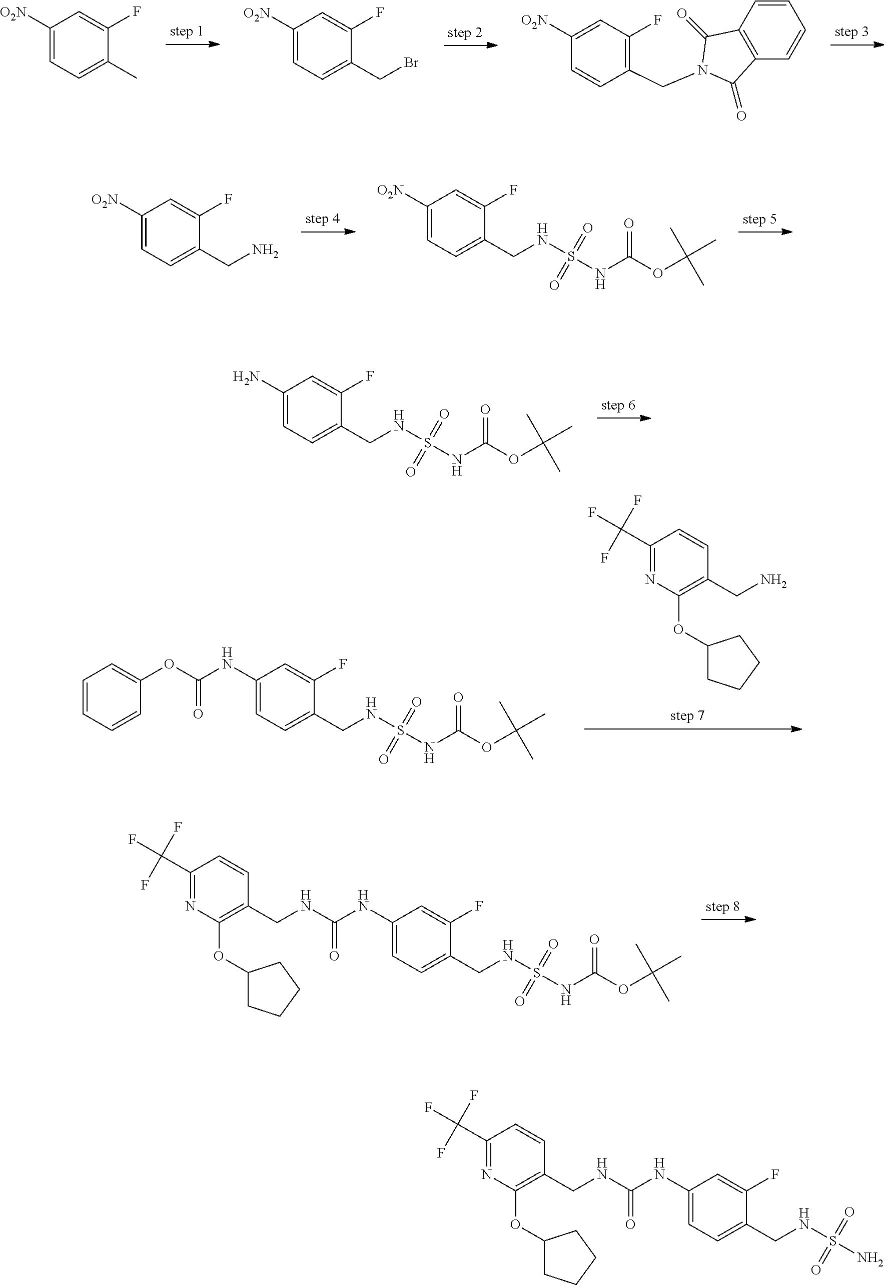

Step 1-6: according to example 35.

Step 7: Tert-butyl N-(2-fluoro-4-(phenylcarbamate)methyl)ureido)benzyl)sulfamoylcarbamate (100 mg, 0.228 mmol) was dissolved in acetonitrile. (2-(Cyclopentyloxy)-6-(trifluoromethyl)pyridin-3-yl)methanamine (50 mg, 0.228 mmol) and 4-dimethylaminopyridine (28 mg) were added to the solution. The reaction mixture was stirred for overnight at 50° C. The mixture was extracted with ethyl acetate and washed with brine. Drying over magnesium sulfate, evaporation of the ethyl acetate and purification by column chromatography tert-butyl N-(4-(3-((2-(cyclopentyloxy)-6-(trifluoromethyl)pyridin-3-yl)methyl)ureido)-2-fluorobenzyl)sulfamoylcarbamate (90 mg, 65%).

Step 8: To tert-butyl N-(4-(3-((2-(cyclopentyloxy)-6-(trifluoromethyl)pyridin-3-yl)methyl)ureido)-2-fluorobenzyl)sulfamoylcarbamate (90 mg, 0.148 mmol) in dichloromethane (6 mL) was added trifluoroacetic acid (2 mL) at 0° C. The mixture was stirred for 30 min at 0° C. and for 2 h at room temperature. The mixture was neutralized by sodium bicarbonate to pH 7-8, extracted with ethyl acetate and washed with brine. Drying over magnesium sulfate, evaporation of the ethyl acetate and purification by column chromatography gave 1-{3-fluoro-4-[(sulfamoylamino)methyl]phenyl}-3-{[2-cyclopentyloxy-6-(trifluoromethyl)pyridin-3-yl]methyl}urea (example 38) (50 mg, 67%).

¹H-NMR (300 MHz, CD₃OD) δ 7.75 (d, 1H, J=7.50 Hz, Ar—H), 7.37 (m, 3H, Ar), 7.00 (dd, 1H, J₁=8.25 Hz, J₂=2.01 Hz Ar—H), 5.51 (m, 1H, penthoxy-1H), 4.35 (s, 2H, Ar—CH₂), 4.17 (s, 2H, Ar—CH₂), 2.03 (m, 2H, penthoxy-2H), 1.85 (m, 4H, penthoxy-4H), 1.66 (m, 2H, penthoxy-2H)

Mass spectrometric data are cited hereinafter by way of example for the following exemplary compounds (Table 1):

TABLE 1

| Exemplary | |
|---|---|
| compound | [M + H] |
| 1 | 504.1 |
| 2 | 506.2 |
| 3 | 522.2 |
| 4 | 516.2 |
| 5 | 501.9 |
| 6 | 516.0 |
| 15 | 492.1 |
| 16 | 506.0 |
| 20 | 518.0 |
| 25 | 534.2 |
| 27 | 504.0 |
| 29 | 518.1 |
| 30 | 506.3 |
| 31 | 520.1 |
| 32 | 462.0 |
| 33 | 476.4 |
| 34 | 488.1 |
| 35 | 466.2 |
| 36 | 480.1 |
| 37 | 494.2 |
| 38 | 506.0 |
| 43 | 547.9 |
| 44 | 549.2 |
| 45 | 554.1 |
| 46 | 500.1 |
| 47 | 516.1 |

### Pharmacological Methods

### I. Functional Testing Carried Out on the Vanilloid Receptor 1 (VR1/TRPV1 Receptor)

The agonistic or antagonistic effect of the substances to be tested on the rat-species vanilloid receptor 1 (VR1/TRPV1) can be determined using the following assay. In this assay, the influx of Ca²⁺ through the receptor channel is quantified with the aid of a Ca²⁺-sensitive dye (type Fluo-4, Molecular Probes Europe BV, Leiden, the Netherlands) in a fluorescent imaging plate reader (FLIPR, Molecular Devices, Sunnyvale, USA).

### Method:

Complete medium: 50 mL HAMS F12 nutrient mixture (Gibco Invitrogen GmbH, Karlsruhe, Germany) with 10% by volume of FCS (foetal calf serum, Gibco Invitrogen GmbH, Karlsruhe, Germany, heat-inactivated); 2 mM L-glutamine (Sigma, Munich, Germany); 1% by weight of AA solution (antibiotic/antimyotic solution, PAA, Pasching, Austria) and 25 ng/mL NGF medium (2.5 S, Gibco Invitrogen GmbH, Karlsruhe, Germany)

Cell culture plate: Poly-D-lysine-coated, black 96-well plates having a clear base (96-well black/clear plate, BD Biosciences, Heidelberg, Germany) are additionally coated with laminin (Gibco Invitrogen GmbH, Karlsruhe, Germany), the laminin being diluted with PBS (Ca—Mg-free PBS, Gibco Invitrogen GmbH, Karlsruhe, Germany) to a concentration of 100 μg/mL. Aliquots having a laminin concentration of 100 μg/mL are removed and stored at −20° C. The aliquots are diluted with PBS in a ratio of 1:10 to 10 μg/mL of laminin and respectively 50 μL of the solution are pipetted into a recess in the cell culture plate. The cell culture plates are incubated for at least two hours at 37° C., the excess solution is removed by suction and the recesses are each washed twice with PBS. The coated cell culture plates are stored with excess PBS which is not removed until just before the feeding of the cells.

### Preparation of the Cells:

The vertebral column is removed from decapitated rats and placed immediately into cold HBSS buffer (Hank's buffered saline solution, Gibco Invitrogen GmbH, Karlsruhe, Germany), i.e. buffer located in an ice bath, mixed with 1% by volume (percent by volume) of an AA solution (antibiotic/antimyotic solution, PAA, Pasching, Austria). The vertebral column is cut longitudinally and removed together with fasciae from the vertebral canal. Subsequently, the dorsal root ganglia (DRG) are removed and again stored in cold HBSS buffer mixed with 1% by volume of an AA solution. The DRG, from which all blood remnants and spinal nerves have been removed, are transferred in each case to 500 μL of cold type 2 collagenase (PAA, Pasching, Austria) and incubated for 35 minutes at 37° C. After the addition of 2.5% by volume of trypsin (PAA, Pasching, Austria), incubation is continued for 10 minutes at 37° C. After complete incubation, the enzyme solution is carefully pipetted off and 500 μL of complete medium are added to each of the remaining DRG. The DRG are respectively suspended several times, drawn through cannulae No. 1, No. 12 and No. 16 using a syringe and transferred to a 50 mL Falcon tube which is filled up to 15 mL with complete medium. The contents of each Falcon tube are respectively filtered through a 70 μm Falcon filter element and centrifuged for 10 minutes at 1,200 rpm and room temperature. The resulting pellet is respectively taken up in 250 μL of complete medium and the cell count is determined.

The number of cells in the suspension is set to 3×10⁵ per mL and 150 μL of this suspension are in each case introduced into a recess in the cell culture plates coated as described hereinbefore. In the incubator the plates are left for two to three days at 37° C., 5% by volume of CO₂ and 95% relative humidity. Subsequently, the cells are loaded with 2 μM of Fluo-4 and 0.01% by volume of Pluronic F127 (Molecular Probes Europe BV, Leiden, the Netherlands) in HBSS buffer (Hank's buffered saline solution, Gibco Invitrogen GmbH, Karlsruhe, Germany) for 30 min at 37° C., washed 3 times with HBSS buffer and after further incubation for 15 minutes at room temperature used for Ca²⁺ measurement in a FLIPR assay. The Ca²⁺ dependent fluorescence is in this case measured before and after the addition of substances (λex=488 nm, λem=540 nm). Quantification is carried out by measuring the highest fluorescence intensity (FC, fluorescence counts) over time.

### FLIPR Assay:

The FLIPR protocol consists of 2 substance additions. First the compounds to be tested (10 μM) are pipetted onto the cells and the Ca²⁺ influx is compared with the control (capsaicin 10 μM). This provides the result in % activation based on the Ca²⁺ signal after the addition of 10 μM of capsaicin (CP). After 5 minutes' incubation, 100 nM of capsaicin are applied and the Ca²⁺ influx is also determined.

Desensitising agonists and antagonists lead to suppression of the Ca²⁺ influx. The % inhibition is calculated compared to the maximum achievable inhibition with 10 μM of capsazepine.

Triple analyses (n=3) are carried out and repeated in at least 3 independent experiments (N=4).

Starting from the percentage displacement caused by different concentrations of the compounds to be tested of general formula I, IC₅₀ inhibitory concentrations which cause a 50-percent displacement of capsaicin were calculated. Kᵢ values for the test substances were obtained by conversion by means of the Cheng-Prusoff equation (Cheng, Prusoff; Biochem. Pharmacol. 22, 3099-3108, 1973).

### Pharmacological Data

The affinity of the compounds according to the invention for the vanilloid receptor 1 (VR1/TRPV1 receptor) was determined as described hereinbefore (pharmacological method I).

The compounds according to the invention display outstanding affinity to the VR1/TRPV1 receptor (Table 2).

In Table 2 the abbreviations below have the following meanings:

Cap=capsaicin

The value after the “@” symbol indicates the concentration at which the inhibition (as a percentage) was respectively determined.

TABLE 2

| Compound | (f) Kᵢ |
|---|---|
| according to | (human being) |
| Example | [nM] Cap |
| 1 | 14 |
| 2 | 18%@5 μM |
| 3 | 13%@5 μM |
| 4 | 12 |
| 5 | 13 |
| 6 | 15 |
| 15 | 37 |
| 16 | 8 |
| 20 | 13 |
| 25 | 111 |
| 27 | 15%@1 μM |
| 29 | 61 |
| 30 | 70 |
| 31 | 15 |
| 32 | 10 |
| 33 | 8 |
| 34 | 1 |
| 35 | 16 |
| 36 | 6 |
| 37 | 7 |
| 38 | 5 |
| 43 | 44 |
| 44 | 83 |
| 45 | 59 |
| 46 | 46 |
| 47 | 41 |

1. A substituted compound of general formula (I), 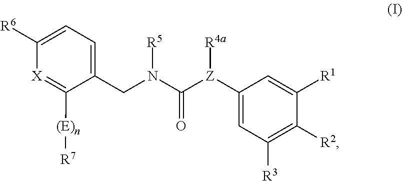
wherein
one of residues R¹ and R² denotes CH₂—N(R⁸)—S(═O)₂—R⁹,
wherein R⁸ represents H, CH₃ or C₂H₅, and
wherein R⁹ represents NH₂, CH₃ or C₂H₅,
and the respective remaining residue of R¹ and R² is selected from the group consisting of H, F, Cl, Br, I, CH₃, CH₂—OH, CH₂—CH₂—OH, CH₂—O—CH₃, CH₂—CH₂—O—CH₃, CF₃, OH, O—CH₃, O—CH₂—OH, O—CH₂—O—CH₃, O—C₂H₅, O—CH₂—CH₂—OH, O—CH₂—CH₂—O—CH₃ and NH₂,
R³ is selected from the group consisting of H, F, Cl, Br, I, CH₃, CF₃, OH, O—CH₃, O—CF₃, and NH₂,
Z represents N or C—R^{4b},
wherein R^{4b} represents H or CH₃,
R^{4a} represents H or CH₃,
R⁵ represents H or CH₃,
X represents N or CH;
R⁶ represents CF₃, an unsubstituted, saturated C₁₋₄ aliphatic residue or an unsubstituted, saturated C₃₋₆ cycloaliphatic residue,
n denotes 0 or 1,
E represents a C₁₋₄ aliphatic group, (C₁₋₄ aliphatic group)-O, (C₁₋₄ aliphatic group)-O—(C₁₋₄ aliphatic group), (C₁₋₄ aliphatic group)-O—(C₁₋₄ aliphatic group)-O, O, an O—C₁₋₄ aliphatic group, O—(C₁₋₄ aliphatic group)-O, O—(C₁₋₄ aliphatic group)-S, S, a S—C₁₋₄ aliphatic group, S—(C₁₋₄ aliphatic group)-S, or S—(C₁₋₄ aliphatic group)-O,
R⁷ represents a C₁₋₄ aliphatic residue, wherein the C₁₋₄ aliphatic residue can be unsubstituted or mono-, di- or trisubstituted with 1, 2 or 3 substituents selected independently of one another from the group consisting of F, Cl, Br, I, OH, O—CH₃, O—CH₂—OH, O—CH₂—O—CH₃, O—C₂H₅, O—CH₂—CH₂—OH, O—CH₂—CH₂—O—CH₃, O—CF₃, NH₂, NH(CH₃), and N(CH₃)₂,
a C₃₋₆ cycloaliphatic residue or a 3 to 6 membered heterocycloaliphatic residue, in each case unsubstituted or mono-, or di-, or trisubstituted with 1, 2 or 3 substituents selected independently of one another from the group consisting of F, Cl, Br, I, CH₃, C₂H₅, CH₂—OH, CH₂—CH₂—OH, CH₂—O—CH₃, CH₂—CH₂—O—CH₃, CH₂—NH(CH₃), CH₂—N(CH₃)₂, CF₃, OH, O—CH₃, O—CH₂—OH, O—CH₂—O—CH₃, O—C₂H₅, O—CH₂—CH₂—OH, O—CH₂—CH₂—O—CH₃, NH₂, NH(CH₃), and N(CH₃)₂, and wherein said C₃₋₆ cycloaliphatic residue and said 3 to 6 membered heterocycloaliphatic residue can in each case optionally be condensed with an unsubstituted phenyl,
a phenyl, or a 5 or 6 membered monocyclic heteroaryl, in each case independently of one another unsubstituted or mono-, or di- or trisubstituted with 1, 2 or 3 substituents selected independently of one another from the group consisting of F, Cl, Br, I, CH₃, C₂H₅, CH₂—OH, CH₂—CH₂—OH, CH₂—O—CH₃, CH₂—CH₂—O—CH₃, CH₂—NH(CH₃), CH₂—N(CH₃)₂, CF₃, OH, O—CH₃, O—CH₂—OH, O—CH₂—O—CH₃, O—C₂H₅, O—CH₂—CH₂—OH, O—CH₂—CH₂—O—CH₃, O—CF₃, SH, S—CH₃, S—CF₃, NH₂, NH(CH₃), and N(CH₃)₂,
with the proviso that n is 1, if R⁷ represents phenyl, a 6 membered monocyclic heteroaryl or a 3 to 6 membered heterocycloaliphatic residue;
or a phenyl, which is condensed with a further ring selected from the group consisting of a C₃₋₆ cycloaliphatic residue, a 3 to 6 membered heterocycloaliphatic residue, a phenyl and a 5 or 6 membered monocyclic heteroaryl to form a bicyclic ring system, wherein said ring system is unsubstituted or mono-, or di- or trisubstituted with 1, 2 or 3 substituents selected independently of one another from the group consisting of F, Cl, Br, I, CH₃, C₂H₅, CH₂—OH, CH₂—CH₂—OH, CH₂—O—CH₃, CH₂—CH₂—O—CH₃, CH₂—NH(CH₃), CH₂—N(CH₃)₂, CF₃, OH, O—CH₃, O—CH₂—OH, O—CH₂—O—CH₃, O—C₂H₅, O—CH₂—CH₂—OH, O—CH₂—CH₂—O—CH₃, O—CF₃, S—CF₃, NH₂, NH(CH₃), and N(CH₃)₂;
in which an “aliphatic group” and an “aliphatic residue” can in each case, independently of one another, be branched or unbranched, saturated or unsaturated, if not indicated otherwise;
in which a “cycloaliphatic residue” and a “heterocycloaliphatic residue” can in each case, independently of one another, be saturated or unsaturated, if not indicated otherwise;
optionally in the form of a single stereoisomer or a mixture of stereoisomers, in the form of the free compound and/or a physiologically acceptable salt thereof.

2. The substituted compound according to claim 1, wherein
one of residues R¹ and R² denotes CH₂—N(R⁸)—S(═O)₂—R⁹,
wherein R⁸ represents H, CH₃, or C₂H₅, and
wherein R⁹ represents NH₂, CH₃, or C₂H₅,
and the respective remaining residue of R¹ and R² is selected from the group consisting of H, F, Cl, Br, I, CH₃, CH₂—OH, CH₂—O—CH₃, CF₃, OH, and O—CH₃.

3. The substituted compound according to claim 1, wherein
R² denotes CH₂—N(R⁸)—S(═O)₂—R⁹,
wherein R⁸ represents H, CH₃, or C₂H₅, and
wherein R⁹ represents NH₂, CH₃, or C₂H₅,
and R¹ is selected from the group consisting of H, F, Cl, Br, I, CH₃, CH₂—OH, CH₂—O—CH₃, CF₃, OH, and O—CH₃.

4. The substituted compound according to claim 1, wherein
R³ is selected from the group consisting of H, F, Cl, CH₃, CF₃, OH and O—CH₃.

5. The substituted compound according to claim 1, wherein
Z represents N and
R^{4a} represents H,
or
Z represents C—R^{4b},
wherein R^{4b} represents H or CH₃, and
R^{4a} represents H.

6. The substituted compound according to claim 1, wherein
R⁵ represents H.

7. The substituted compound according to claim 1, wherein
X represents N.

8. The substituted compound according to claim 1, wherein
R⁶ represents CF₃, tert.-Butyl or cyclopropyl.

9. The substituted compound according to claim 1, wherein
n denotes 0 or 1,
E is selected from the group consisting of CH₂, CH₂—CH₂, CH₂—CH₂—CH₂, CH═CH, C≡C, CH₂—O, CH₂—CH₂—O, CH₂—CH₂—CH₂—O, CH₂—O—CH₂, CH₂—CH₂—O—CH₂, CH₂—CH₂—CH₂—O—CH₂, CH₂—O—CH₂—CH₂, CH₂—CH₂—O—CH₂—CH₂, CH₂—CH₂—CH₂—O—CH₂—CH₂, CH₂—O—CH₂—CH₂—CH₂, CH₂—CH₂—O—CH₂—CH₂—CH₂, CH₂—CH₂—CH₂—O—CH₂—CH₂—CH₂, CH₂—O—CH₂—O, CH₂—CH₂—O—CH₂—O, CH₂—CH₂—CH₂—O—CH₂—O, CH₂—O—CH₂—CH₂—O, CH₂—CH₂—O—CH₂—CH₂—O, CH₂—CH₂—CH₂—O—CH₂—CH₂—O, CH₂—O—CH₂—CH₂—CH₂—O, CH₂—CH₂—O—CH₂—CH₂—CH₂—O, CH₂—CH₂—CH₂—O—CH₂—CH₂—CH₂—O, O, O—CH₂, O—CH₂—CH₂, O—CH₂—CH₂—CH₂, O—CH₂—, O—CH₂—CH₂—, O—CH₂—CH₂—CH₂—O, O—CH₂—S, O—CH₂—CH₂—S, O—CH₂—CH₂—CH₂—S, S, S—CH₂, S—CH₂—CH₂, S—CH₂—CH₂—CH₂, S—CH₂—O, S—CH₂—CH₂—O, and S—CH₂—CH₂—CH₂—O,
R⁷ represents a C₁₋₄ aliphatic residue, wherein the C₁₋₄ aliphatic residue can be unsubstituted or mono-, di- or trisubstituted with 1, 2 or 3 substituents selected independently of one another from the group consisting of F, Cl, OH, and O—CH₃,
a C₃₋₆ cycloaliphatic residue or a 3 to 6 membered heterocycloaliphatic residue, in each case unsubstituted or mono-, or di-, or trisubstituted with 1, 2 or 3 substituents selected independently of one another from the group consisting of F, Cl, CH₃, C₂H₅, CH₂—OH, CF₃, OH, O—CH₃, NH₂, NH(CH₃), and N(CH₃)₂,
or an unsubstituted cycloaliphatic residue, which is condensed with an unsubstituted phenyl,
phenyl, or a 5 or 6 membered monocyclic heteroaryl, in each case independently of one another unsubstituted or mono-, or di- or trisubstituted with 1, 2 or 3 substituents selected independently of one another from the group consisting of F, Cl, CH₃, C₂H₅, CF₃, OH, O—CH₃, and O—CF₃,
with the proviso that n is 1, if R⁷ represents phenyl, a 6 membered monocyclic heteroaryl or a 3 to 6 membered heterocycloaliphatic residue;
or a phenyl which is condensed with a further ring selected from the group consisting of a C₃₋₆ cycloaliphatic residue and a 3 to 6 membered heterocycloaliphatic residue to form a bicyclic ring system, wherein said ring system is unsubstituted or mono-, or di- or trisubstituted with 1, 2 or 3 substituents selected independently of one another from the group consisting of F, Cl, CH₃, C₂H₅, CF₃, OH, O—CH₃, and O—CF₃.

10. The substituted compound according to claim 1, wherein
n denotes 0 or 1,
E is selected from the group consisting of CH₂, CH₂—CH₂, CH₂—CH₂—CH₂, CH═CH, C≡C, CH₂—O, CH₂—CH₂—O, CH₂—CH₂—CH₂—O, CH₂—O—CH₂, CH₂—CH₂—O—CH₂, CH₂—CH₂—O—CH₂, CH₂—O—CH₂—CH₂, CH₂—CH₂—O—CH₂—CH₂, CH₂CH₂—CH₂—O—CH₂—CH₂, CH₂—O—CH₂—CH₂—CH₂, CH₂—CH₂—O—CH₂CH₂—CH₂, CH₂—CH₂—CH₂—O—CH₂—CH₂CH₂, CH₂—O—CH₂—, CH₂—CH₂—O—CH₂—, CH₂—CH₂—CH₂—O—CH₂—, CH₂—O—CH₂—CH₂—O, CH₂—CH₂—O—CH₂—CH₂—O, CH₂—CH₂—CH₂—O—CH₂—CH₂—O, CH₂—O—CH₂—CH₂—CH₂—O, CH₂—CH₂—O—CH₂—CH₂—CH₂—O, CH₂—CH₂—CH₂—O—CH₂—CH₂—CH₂—O, O, O—CH₂, O—CH₂—CH₂, O—CH₂—CH₂—CH₂, O—CH₂—O, O—CH₂—CH₂—O, O—CH₂—CH₂—CH₂—O, O—CH₂—S, O—CH₂—CH₂—S, O—CH₂—CH₂—CH₂—S, S, S—CH₂, S—CH₂—CH₂, S—CH₂—CH₂—CH₂, S—CH₂—O, S—CH₂—CH₂—O, and S—CH₂—CH₂—CH₂—O,
R⁷ represents an unsubstituted C₁₋₄ aliphatic residue,
cyclopropyl, cyclobutyl, cyclopentyl, cyclohexyl, cyclopentenyl, cyclohexenyl, dihydroindenyl, piperidinyl, pyrrolidinyl, piperazinyl, morpholinyl or tetrahydropyranyl, in each case independently of one another unsubstituted or mono-, or disubstituted with 1 or 2 substituents selected independently of one another from the group consisting of F, Cl, CH₃, OH, and O—CH₃;
phenyl, unsubstituted or mono-, or di- or trisubstituted with 1, 2 or 3 substituents selected independently of one another from the group consisting of F, Cl, CH₃, CF₃, OH, O—CH₃, and O—CF₃,
furyl, thienyl, oxazolyl, isooxazolyl or thiazolyl, unsubstituted or mono-, or disubstituted with 1 or 2 substituents selected independently of one another from the group consisting of F, Cl, CH₃, CF₃, OH, O—CH₃, and O—CF₃,
or pyridyl or pyrimidinyl, unsubstituted or mono-, or disubstituted with 1 or 2 substituents selected independently of one another from the group consisting of F, Cl, CH₃, CF₃, OH, O—CH₃, and O—CF₃,
with the proviso that n is 1, if R⁷ represents phenyl, pyridyl, pyrimidinyl, piperidinyl, pyrrolidinyl, piperazinyl, morpholinyl or tetrahydropyranyl;
or a phenyl, which is condensed with a dioxolanyl, dioxanyl, or a dihydropyrrolyl to form a bicyclic ring system selected from the group consisting of benzodioxolanyl, benzodioxanyl, indolyl and isoindolyl, wherein said ring system is unsubstituted.

11. The substituted compound according to claim 1, wherein
one of residues R¹ and R² denotes CH₂—N(R⁸)—S(═O)₂—R⁹,
wherein R⁸ represents H, CH₃, or C₂H₅, and
wherein R⁹ represents NH₂, CH₃, or C₂H₅,
and the respective remaining residue of R¹ and R² is selected from the group consisting of H, F, Cl, Br, I, CH₃, CH₂—OH, CH₂—O—CH₃, CF₃, OH, and O—CH₃,
R³ is selected from the group consisting of H, F, Cl, CH₃, and O—CH₃,
Z represents N and
R^{4a} represents H,
or
Z represents C—R^{4b},
wherein R^{4b} represents H or CH₃, and
R^{4a} represents H,
R⁵ represents H,
X represents N or CH,
R⁶ represents CF₃, tert.-Butyl or cyclopropyl,
n denotes 0 or 1,
E is selected from the group consisting of CH₂, CH₂—CH₂, CH═CH, C≡C, CH₂—O, CH₂—CH₂—O, CH₂—CH₂—CH₂—O, CH₂—O—CH₂, CH₂—CH₂—O—CH₂, CH₂—CH₂—CH₂—O—CH₂, CH₂—O—CH₂—CH₂, CH₂—CH₂—O—CH₂—CH₂, CH₂CH₂—CH₂—O—CH₂—CH₂, CH₂—O—CH₂—CH₂—CH₂, CH₂—CH₂—O—CH₂—CH₂—CH₂, CH₂—CH₂—CH₂—O—CH₂—CH₂—CH₂, CH₂—O—CH₂—O, CH₂—CH₂—O—CH₂—O, CH₂—CH₂—CH₂—O—CH₂—O, CH₂—O—CH₂—CH₂—O, CH₂—CH₂—O—CH₂—CH₂—O, CH₂—CH₂—CH₂—O—CH₂—CH₂—O, CH₂—O—CH₂—CH₂—CH₂—O, CH₂—CH₂—O—CH₂—CH₂—CH₂—O, CH₂—CH₂—CH₂—O—CH₂—CH₂—CH₂—O, O, O—CH₂, O—CH₂—CH₂, O—CH₂—CH₂—CH₂, S, S—CH₂, S—CH₂—CH₂, S—CH₂—CH₂—CH₂, S—CH₂—O, S—CH₂—CH₂—O, and S—CH₂—CH₂—CH₂—O,
R⁷ represents methyl, ethyl, n-propyl, 2-propyl, n-butyl, or tert.-butyl,
cyclopropyl, cyclopentyl, cyclohexyl, cyclohexenyl, dihydroindenyl, piperidinyl, pyrrolidinyl, morpholinyl or tetrahydropyranyl, in each case independently of one another unsubstituted or mono-, or disubstituted with 1 or 2 substituents selected independently of one another from the group consisting of F, Cl, and CH₃;
an unsubstituted phenyl,
furyl, thienyl, oxazolyl, isooxazolyl or thiazolyl, in each case unsubstituted,
with the proviso that n is 1, if R⁷ represents an unsubstituted phenyl, piperidinyl, pyrrolidinyl, morpholinyl or tetrahydropyranyl;
or a phenyl, which is condensed with a dioxolanyl or a dihydropyrrolyl to form a bicyclic ring system selected from the group consisting of benzodioxolanyl and indolyl, wherein said ring system is unsubstituted.

12. The substituted compound according to claim 1 selected from the group consisting of
1 2-(3-fluoro-4-(methylsulfonamidomethyl)phenyl)-N-((2-pentyl-6-(trifluoromethyl)pyridin-3-yl)methyl)propanamide;
2 2-(3-fluoro-4-(methylsulfonamidomethyl)phenyl)-N-((2-(3-methoxypropyl)-6-(trifluoromethyl)pyridin-3-yl)methyl)propanamide;
3 2-(3-fluoro-4-(methylsulfonamidomethyl)phenyl)-N-((2-((2-methoxyethoxy)methyl)-6-(trifluoromethyl)pyridin-3-yl)methyl)propanamide;
4 (E)-N-((2-(3,3-dimethylbut-1-enyl)-6-(trifluoromethyl)pyridin-3-yl)methyl)-2-(3-fluoro-4-(methylsulfonamidomethyl)phenyl)propanamide;
5 N-((2-cyclopentyl-6-(trifluoromethyl)pyridin-3-yl)methyl)-2-(3-fluoro-4-(methylsulfonamidomethyl)phenyl)propanamide;
6 N-((2-cyclohexyl-6-(trifluoromethyl)pyridin-3-yl)methyl)-2-(3-fluoro-4-(methylsulfonamidomethyl)phenyl)propanamide;
7 N-((2-(4,4-difluorocyclohexyl)-6-(trifluoromethyl)pyridin-3-yl)methyl)-2-(3-fluoro-4-(methylsulfonamidomethyl)phenyl)propanamide;
8 N-((2-cyclohexenyl-6-(trifluoromethyl)pyridin-3-yl)methyl)-2-(3-fluoro-4-(methylsulfonamidomethyl)phenyl)propanamide;
9 N-((2-(cyclohexylmethyl)-6-(trifluoromethyl)pyridin-3-yl)methyl)-2-(3-fluoro-4-(methylsulfonamidomethyl)phenyl)propanamide;
10 2-(3-fluoro-4-(methylsulfonamidomethyl)phenyl)-N-((2-(piperidin-1-ylmethyl)-6-(trifluoromethyl)pyridin-3-yl)methyl)propanamide;
11 N-((2-benzyl-6-(trifluoromethyl)pyridin-3-yl)methyl)-2-(3-fluoro-4-(methylsulfonamidomethyl)phenyl)propanamide;
12 2-(3-fluoro-4-(methylsulfonamidomethyl)phenyl)-N-((2-phenethyl-6-(trifluoromethyl)pyridin-3-yl)methyl)propanamide;
13 (E)-2-(3-fluoro-4-(methylsulfonamidomethyl)phenyl)-N-((2-styryl-6-(trifluoromethyl)pyridin-3-yl)methyl)propanamide;
14 2-(3-fluoro-4-(methylsulfonamidomethyl)phenyl)-N-((2-(phenylethynyl)-6-(trifluoromethyl)pyridin-3-yl)methyl)propanamide;
15 2-(3-fluoro-4-(methylsulfonamidomethyl)phenyl)-N-((2-isopropoxy-6-(trifluoromethyl)pyridin-3-yl)methyl)propanamide;
16 N-((2-butoxy-6-(trifluoromethyl)pyridin-3-yl)methyl)-2-(3-fluoro-4-(methylsulfonamidomethyl)phenyl)propanamide;
17 N-((2-(cyclopropylmethoxy)-6-(trifluoromethyl)pyridin-3-yl)methyl)-2-(3-fluoro-4-(methylsulfonamidomethyl)phenyl)propanamide;
18 N-((2-(cyclohexylmethoxy)-6-(trifluoromethyl)pyridin-3-yl)methyl)-2-(3-fluoro-4-(methylsulfonamidomethyl)phenyl)propanamide;
19 N-(2-(cyclopentyloxy)-4-(trifluoromethyl)benzyl)-2-(3-fluoro-4-(methylsulfonamidomethyl)phenyl)propanamide;
20 N-((2-(cyclopentyloxy)-6-(trifluoromethyl)pyridin-3-yl)methyl)-2-(3-fluoro-4-(methylsulfonamidomethyl)phenyl)propanamide;
21 N-(4-tert-butyl-2-(cyclopentyloxy)benzyl)-2-(3-fluoro-4-(methylsulfonamidomethyl)phenyl)propanamide;
22 N-((6-tert-butyl-2-(cyclopentyloxy)pyridin-3-yl)methyl)-2-(3-fluoro-4-(methylsulfonamidomethyl)phenyl)propanamide;
23 N-((2-(cyclopentyloxy)-6-cyclopropylpyridin-3-yl)methyl)-2-(3-fluoro-4-(methylsulfonamidomethyl)phenyl)propanamide;
24 N-((2-(2,3-dihydro-1H-inden-2-yloxy)-6-(trifluoromethyl)pyridin-3-yl)methyl)-2-(3-fluoro-4-(methylsulfonamidomethyl)phenyl)propanamide;
25 2-(3-fluoro-4-(methylsulfonamidomethyl)phenyl)-N-((2-(tetrahydro-2H-pyran-4-yloxy)-6-(trifluoromethyl)pyridin-3-yl)methyl)propanamide;
26 2-(3-fluoro-4-(methylsulfonamidomethyl)phenyl)-N-((2-phenoxy-6-(trifluoromethyl)pyridin-3-yl)methyl)propanamide;
27 N-((2-isopropoxy-6-(trifluoromethyl)pyridin-3-yl)methyl)-2-(3-methoxy-4-(methylsulfonamidomethyl)phenyl)propanamide;
28 N-((2-butoxy-6-(trifluoromethyl)pyridin-3-yl)methyl)-2-(3-hydroxy-4-(methylsulfonamidomethyl)phenyl)propanamide;
29 N-((2-butoxy-6-(trifluoromethyl)pyridin-3-yl)methyl)-2-(3-methoxy-4-(methylsulfonamidomethyl)phenyl)propanamide;
30 2-(4-(ethylsulfonamidomethyl)-3-fluorophenyl)-N-((2-isopropoxy-6-(trifluoromethyl)pyridin-3-yl)methyl)propanamide;
31 N-((2-butoxy-6-(trifluoromethyl)pyridin-3-yl)methyl)-2-(4-(ethylsulfonamidomethyl)-3-fluorophenyl)propanamide;
32 1-{[2-isopropoxy-6-(trifluoromethyl)pyridin-3-yl]methyl}-3-{4-[(sulfamoylamino)methyl]phenyl}urea;
33 1-{[2-butoxy-6-(trifluoromethyl)pyridin-3-yl]methyl}-3-{4-[(sulfamoylamino)methyl]phenyl}urea;
34 1-{[2-cyclopentyloxy-6-(trifluoromethyl)pyridin-3-yl]methyl}-3-{4-[(sulfamoylamino)methyl]phenyl}urea;
35 1-{3-fluoro-4-[(sulfamoylamino)methyl]phenyl}-3-{[2-ethoxy-6-(trifluoromethyl)pyridin-3-yl]methyl}urea;
36 1-{3-fluoro-4-[(sulfamoylamino)methyl]phenyl}-3-{[2-isopropoxy-6-(trifluoromethyl)pyridin-3-yl]methyl}urea;
37 1-{3-fluoro-4-[(sulfamoylamino)methyl]phenyl}-3-{[2-butoxy-6-(trifluoromethyl)pyridin-3-yl]methyl}urea;
38 1-{3-fluoro-4-[(sulfamoylamino)methyl]phenyl}-3-{[2-cyclopentyloxy-6-(trifluoromethyl)pyridin-3-yl]methyl}urea;
39 N-((2-(butylthio)-6-(trifluoromethyl)pyridin-3-yl)methyl)-2-(3-fluoro-4-(methylsulfonamidomethyl)phenyl)propanamide;
40 2-(3-fluoro-4-(methylsulfonamidomethyl)phenyl)-N-((2-(3-(4-methylpiperidin-1-yl)propylthio)-6-(trifluoromethyl)pyridin-3-yl)methyl)propanamide;
41 N-((2-(cyclopentylthio)-6-(trifluoromethyl)pyridin-3-yl)methyl)-2-(3-fluoro-4-(methylsulfonamidomethyl)phenyl)propanamide;
42 2-(3-fluoro-4-(methylsulfonamidomethyl)phenyl)-N-((2-(2-phenoxyethylthio)-6-(trifluoromethyl)pyridin-3-yl)methyl)propanamide;
43 N-((2-(cyclohexylthio)-6-(trifluoromethyl)pyridin-3-yl)methyl)-2-(3-fluoro-4-(methylsulfonamidomethyl)phenyl)propanamide;
44 N-((2-(1H-indol-6-yl)-6-(trifluoromethyl)pyridin-3-yl)methyl)-2-(3-fluoro-4-(methylsulfonamidomethyl)phenyl)propanamide;
45 N-((2-(benzo[d][1,3]dioxol-5-yl)-6-(trifluoromethyl)pyridin-3-yl)methyl)-2-(3-fluoro-4-(methylsulfonamidomethyl)phenyl)propanamide;
46 2-(3-fluoro-4-(methylsulfonamidomethyl)phenyl)-N-((2-(furan-3-yl)-6-(trifluoromethyl)pyridin-3-yl)methyl)propanamide;
47 2-(3-fluoro-4-(methylsulfonamidomethyl)phenyl)-N-((2-(thiophen-2-yl)-6-(trifluoromethyl)pyridin-3-yl)methyl)propanamide;
48 2-(3-fluoro-4-(methylsulfonamidomethyl)phenyl)-N-((2-(thiophen-3-yl)-6-(trifluoromethyl)pyridin-3-yl)methyl)propanamide; and
49 2-(3-fluoro-4-(methylsulfonamidomethyl)phenyl)-N-((2-(thiazol-4-yl)-6-(trifluoromethyl)pyridin-3-yl)methyl)propanamide;
optionally in the form of a single stereoisomer or a mixture of stereoisomers, in the form of the free compound and/or a physiologically acceptable salt thereof.

13. A pharmaceutical composition comprising at least one substituted compound according to claim 1.

14. A method for treating and/or preventing a disorder or disease selected from the group consisting of pain; hyperalgesia; allodynia; causalgia; migraine; depression; nervous affection; an axonal injury; a neurodegenerative disease; a cognitive dysfunction; epilepsy; a respiratory disease; a cough; urinary incontinence; overactive bladder (OAB); a disorder and/or injury of the gastrointestinal tract; a duodenal ulcer; a gastric ulcer; irritable bowel syndrome; a stroke; an eye irritation; a skin irritation; a neurotic skin disease; an allergic skin disease; psoriasis; vitiligo; herpes simplex; an inflammation; diarrhoea; pruritus; osteoporosis; arthritis; osteoarthritis; a rheumatic disease; an eating disorder; medication dependency; misuse of medication; withdrawal symptoms in medication dependency; development of tolerance to medication; drug dependency; misuse of drugs; withdrawal symptoms in drug dependency; alcohol dependency; misuse of alcohol and withdrawal symptoms in alcohol dependency; for diuresis; for antinatriuresis; for influencing the cardiovascular system; for increasing vigilance; for the treatment of wounds and/or burns; for the treatment of severed nerves; for increasing libido; for modulating movement activity; for anxiolysis; for local anaesthesia and/or for inhibiting undesirable side effects triggered by administration of a vanilloid receptor 1 agonist, comprising administering to a mammal an effective amount of at least one compound according to claim 1.

15. The method according to claim 14, wherein the pain is selected from the group consisting of acute pain, chronic pain, neuropathic pain, visceral pain and joint pain.

16. The method according to claim 14, wherein the neurodegenerative disease is selected from the group consisting of multiple sclerosis, Alzheimer's disease, Parkinson's disease and Huntington's disease.

17. The method according to claim 14, wherein the cognitive dysfunction is a cognitive deficiency state.

18. The method according to claim 17, wherein the cognitive dysfunction is a memory disorder.

19. The method according to claim 14, wherein the respiratory disease is selected from the group consisting of asthma, bronchitis and pulmonary inflammation.

20. The method according to claim 14, wherein the inflammation is an inflammation of the intestine, the eyes, the bladder, the skin or the nasal mucous membrane.

21. The method according to claim 14, wherein the eating disorder is selected from the group consisting of bulimia, cachexia, anorexia and obesity.

22. The method according to claim 14, wherein the development of tolerance to medication is development of tolerance to natural or synthetic opioids.

23. The method according to claim 14, wherein the undesirable side effects are selected from the group consisting of hyperthermia, hypertension and bronchoconstriction and the vanilloid receptor 1 agonist is selected from the group consisting of capsaicin, resiniferatoxin, olvanil, arvanil, SDZ-249665, SDZ-249482, nuvanil and capsavanil.
